SEQUENCE LISTING (10 sequences)

SEQ ID NO: 1 - protein, 121 aa, synthetic construct
  REGION 1..121: note = recombinant peptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSS

SEQ ID NO: 2 - protein, 111 aa, synthetic construct
  REGION 1..111: note = recombinant polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIK

SEQ ID NO: 3 - protein, 5 aa, synthetic construct
  REGION 1..5: note = Synthetic peptide
SSWMN

SEQ ID NO: 4 - protein, 17 aa, synthetic construct
  REGION 1..17: note = Synthetic peptide
RIYPGDGDTNYNVKFKG

SEQ ID NO: 5 - protein, 12 aa, synthetic construct
  REGION 1..12: note = Synthetic peptide
SGFITTVRDFDY

SEQ ID NO: 6 - protein, 15 aa, synthetic construct
  REGION 1..15: note = Synthetic peptide
RASESVDTFGISFMN

SEQ ID NO: 7 - protein, 7 aa, synthetic construct
  REGION 1..7: note = Synthetic peptide
EASNQGS

SEQ ID NO: 8 - protein, 9 aa, synthetic construct
  REGION 1..9: note = Synthetic peptide
QQSKEVPFT

SEQ ID NO: 9 - protein, 451 aa, synthetic construct
  REGION 1..451: note = recombinant polypeptide
EVQLVESGGGLVQPGGSLRLSCAASGFTFSSSWMNWVRQAPGKGLEWVGRIYPGDGDTNYNVKFKGRFTISRDDSKNSLYLQMNSLKTEDTAVYYCARSGFITTVRDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 218 aa, synthetic construct
  REGION 1..218: note = recombinant polypeptide
EIVLTQSPDFQSVTPKEKVTITCRASESVDTFGISFMNWFQQKPDQSPKLLIHEASNQGSGVPSRFSGSGSGTDFTLTINSLEAEDAATYYCQQSKEVPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2023/0406928 A1
(43) Pub. Date: Dec. 21, 2023

(54) METHODS OF TREATING NEUROMYELITIS OPTICA SPECTRUM DISORDER

(71) Applicant: Viela Bio, Inc., Deerfield, IL (US)

(72) Inventors: William REES, North Bethesda, MD (US); Eliezer KATZ, Deerfield, IL (US); Michael SMITH, Deerfield, IL (US); Nanette MITTEREDER, Deerfield, IL (US)

(21) Appl. No.: 18/147,287

(22) Filed: Dec. 28, 2022

Related U.S. Application Data
(63) Continuation of application No. PCT/US2021/039911, filed on Jun. 30, 2021.
(60) Provisional application No. 63/071,092, filed on Aug. 27, 2020.
(60) Provisional application No. 63/052,093, filed on Jul. 15, 2020.
(60) Provisional application No. 63/046,133, filed on Jun. 30, 2020.

(51) Int. Cl. C07K 16/28; A61P 25/28; A61K 45/06

(52) U.S. Cl. CPC C07K 16/2809; A61P 25/28; A61K 45/06; A61K 2039/505

(57) ABSTRACT

Methods of treating neuromyelitis optica spectrum disorder (NMOSD) are disclosed herein. In particular, methods of treating NMOSD in a subject identified as having a higher or lower level of NMOSD-disease activity, e.g., by serum glial fibrillary acidic protein concentration, are provided.

### CROSS REFERENCE TO RELATED APPLICATIONS

This application is a continuation of International Application No. PCT/US2021/039911, filed Jun. 30, 2021, which claims the benefit of U.S. Provisional Application No. 63/046,133, filed Jun. 30, 2020; U.S. Provisional Application No. 63/052,093, filed Jul. 15, 2020; and U.S. Provisional Application No. 63/071,092, filed Aug. 27, 2020, each of which is hereby incorporated by reference in its entirety.

### REFERENCE TO AN ELECTRONIC SEQUENCE LISTING

The contents of the electronic sequence listing (HOPA_029_03US_SeqList_ST26.xml; Size: 14,376 bytes; and Date of Creation: Dec. 22, 2022) are herein incorporated by reference in its entirety.

### BACKGROUND

Neuromyelitis Optica Spectrum Disorder (NMOSD) is a rare, chronic, autoimmune, inflammatory disorder of the central nervous system (Cree B A, et al., Mult Scler. 2016; 22(7):862-872). It is typically characterized by recurrent attacks of optic neuritis and longitudinally extensive transverse myelitis, while brain and brainstem inflammation are less frequently observed. Attacks can be severe, and recovery is typically incomplete, thus leading to cumulative disability.

Traditionally, immunosuppressants, such as corticosteroids and mycophenolate mofetil (Trebst C, et al., J Neurol. 2014; 261(1):1-16), and rituximab, a CD20 B-cell-depleting antibody (Cree B A, et al., Neurology. 2005; 64(7):1270-1272; Damato V, et al., JAMA Neurol., 2016; 73(11):1342-1348), are used as maintenance therapeutics to prevent attacks, although clinical evidence for their effectiveness is limited and based on uncontrolled or retrospective studies. A number of new therapies have recently proved to be effective (Pittock S J, et al., N Engl J Med. 2019; 381(7):614-625; Yamamura T, et al., N Engl J Med., 2019; 381(22):2114-2124; Fujihara K., Curr Opin Neural. 2019; 32(3):385-394), including inebilizumab, a humanized affinity-optimized, afucosylated immunoglobulin G1 kappa monoclonal antibody (Chen D, et. al., J Clin Med., 2016; 5(12); Cree B, et al., Lancet, 2019; 394(10206):1352-1363). Inebilizumab binds to the B-cell-specific surface antigen CD19 and depletes a wide range of B cells.

There is a need in the art to further improve treatment options for NMOSD subjects and, in turn, slow or stop the accumulation of damage resulting from their NMOSD-related disease activity including, in particular, NMOSD-related attacks. The ability to identify an NMOSD subject with elevated NMOSD-related disease activity would present clinicians with an opportunity to appropriately adjust or select the subject's NMOSD therapeutic regimen, at a time the subject is more vulnerable to suffering NMOSD-related damage.

### BRIEF SUMMARY

Provided are methods of reducing neuromyelitis optica spectrum disorder (NMOSD)-related damage in a subject in need thereof, comprising administering a composition that comprises Inebilizumab or a derivative thereof to the subject in need thereof, thereby reducing the NMODS-related damage, wherein the subject in need thereof comprises a serum glial fibrillary astrocytic protein (sGFAP) concentration of at least about 160 pg/mL. In aspects, the sGFAP concentration is at least about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater. In aspects, the sGFAP concentration is at least about 170 pg/mL. In aspects, the composition that comprises Inebilizumab or the derivative thereof is administered intravenously. In aspects, the intravenous administration is at a dose of about 300 mg. In aspects, the administering is repeated at least twice. In aspects, the administering is repeated every 6 months. In aspects, the reducing NMOSD-related damage is determined by at least one of: (a) a reduction in a number of NMOSD-related attacks in the subject in need thereof after the administering as compared to a baseline number of NMOSD-related attacks in the subject in need thereof before the administering; or (b) a reduction in a number of NMOSD-related attacks in the subject in need thereof after the administering as compared to an otherwise comparable control subject lacking the administering. In aspects, the baseline number of NMODS-related attacks are determined over a first time period preceding the administering, wherein the number of NMODS-related attacks reduced by the administering are determined over a second time period following the administering, and wherein the first time period and the second time period are of equal length. In aspects, the first time period and the second time period are at least one year. In aspects, the reducing the NMOSD-related damage comprises reducing NMOSD-related attacks that are graded major in severity in the subject in need thereof. In aspects, the reducing the NMOSD-related damage comprises eliminating NMOSD-related attacks that are graded major in severity in the subject in need thereof. In aspects, the reducing the NMOSD-related damage in the subject in need thereof comprises: (a) reducing a number of magnetic resonance imaging (MRI) lesions; (b) reducing rate of increase in new MRI lesions; or (c) both (a) and (b). In aspects, the reducing the NMOSD-related damage in the subject in need thereof comprises: (a) reducing a rate of worsening of expanded disability status scale (EDSS) score; or (b) improving the EDSS score. In aspects, the methods further comprise identifying the subject in need thereof by determining the sGFAP concentration of at least about 160 pg/mL.

Provided are methods of preventing neuromyelitis optica spectrum disorder (NMOSD) relapse in a subject in need thereof, the methods comprising administering a composition that comprises Inebilizumab or a derivative thereof to the subject in need thereof thereby preventing NMODS relapse, wherein the subject in need thereof comprises a serum glial fibrillary acidic protein (sGFAP) concentration of about 165 pg/mL. In some aspects, the sGFAP concentration is about: 166 pg/mL, 167 pg/mL, 168 pg/mL, 169 pg/mL, 170 pg/mL, 171 pg/mL, 172 pg/mL, 173 pg/mL, or greater. In aspects, the sGFAP concentration is about 170 pg/mL. In aspects, the composition that comprises Inebilizumab or the derivative thereof is administered intravenously. In aspects, the intravenous administration is at a dose of about 300 mg. In aspects, the administering is repeated at least twice. In aspects, the administering is repeated every 6 months. In aspects, the preventing lasts for at least 1 year after the administering. In aspects, the preventing lasts for at least 2 years after the administering. In aspects, the administering decreases sGFAP concentration: (a) in the subject in need thereof as compared to sGFAP concentration prior to the administering; (b) in the subject in need thereof as compared to the subject in need thereof's baseline sGFAP concentration; or (c) in an otherwise comparable subject in need thereof lacking the administering. In aspects, the preventing results in a reduction in MRI lesions in the subject in need thereof as determined by: (a) a reduction in a number of the MRI lesions; (b) a reduction in size of the MRI lesions; or (c) both (a) and (b). In aspects, the preventing results in an improvement in EDSS score in the subject in need thereof.

Provided are also methods of suppressing a neuromyelitis optica spectrum disorder (NMOSD)-related attack in a subject diagnosed with NMOSD, comprising: (a) identifying the subject as at-risk for an NMOSD-related attack, wherein the subject is identified as an at-risk subject if the subject comprises an increase in sGFAP concentration relative to a baseline sGFAP concentration; and (b) administering a therapeutic to the at-risk subject in an amount effective to suppress the NMODS-related attack, wherein the administering is performed at most one week following the identifying. In aspects, the increase in the sGFAP concentration comprises an at least 10-fold increase relative to the baseline sGFAP concentration. In aspects, the increase in the sGFAP concentration comprises an at least 20-fold increase relative to the baseline sGFAP concentration. In aspects, the subject at risk for the NMOSD-related attack is not undergoing a treatment for NMOSD that comprises Inebilizumab or a derivative thereof. In aspects, the increase in sGFAP concentration comprises an increase of 50% to 15 (% relative to the baseline sGFAP concentration; wherein the subject at risk for the NMOSD-related attack is undergoing treatment for NMOSD, and wherein the treatment comprises Inebilizumab or a derivative thereof. In aspects, the therapeutic comprises one or more of a steroid, plasmapheresis, immunoadsorption, or a complement inhibitor. In aspects, the therapeutic comprises one or more of Eculizumab, Satralizumab, Ublituximab, Ravulizumab, Rituximab, Azathioprine, Mycophenolate Mofetil, or a low dose corticosteroid. In aspects, the administering is performed at most 24 hours following the identifying. In aspects, the suppressing the NMOSD-related attack comprises: (a) reducing a number of NMODS-related attacks; or (b) preventing a NMOSD-related attack. In aspects, the methods comprise (a), wherein the reducing comprises reducing a number of NMODS-related attacks graded as major in severity. In aspects, the suppressing the NMOSD-related attack comprises a recovery from the NMOSD-related attack that is graded as a major recovery. In aspects, the suppressing the NMOSD-related attack results in a prevention of new MRI lesions in the subject at-risk for an NMOSD-related attack. In aspects, the suppressing the NMOSD-related attack results in a reduction in NMOSD-related disability in the subject at-risk for an NMOSD-related attack. In aspects, the reduction in NMOSD-related disability is a reduction in worsening of the subject at-risk for an NMOSD-related attack's EDSS score. In aspects, the therapeutic comprises Inebilizumab or a derivative thereof.

Provided is also methods of treating neuromyelitis optica spectrum disorder (NMOSD) in a subject in need thereof, comprising administering a therapeutically effective amount of a B cell depleting therapy to the subject in need thereof, wherein the subject has a serum glial fibrillary acidic protein (sGFAP) concentration of about 160 pg/mL. In aspects, the sGFAP concentration is about: 165 pg/mL, 166 pg/mL, 167 pg/mL, 168 pg/mL, 169 pg/mL, 170 pg/mL, 171 pg/mL, 172 pg/mL, 173 pg/mL, or greater. In aspects, the subject in need thereof has a sGFAP concentration of about 170 pg/mL to 171 pg/mL. In aspects, the B cell depleting therapy comprises Inebilizumab or a derivative thereof. In aspects, the therapeutically effective amount of the B cell depleting therapy is about 300 mg.

Provided are methods of reducing neuromyelitis optica spectrum disorder (NMOSD)-related disability in a subject in need thereof, the methods comprising administering a composition that comprises Inebilizumab or a derivative thereof to the subject in need thereof, thereby reducing the NMOSD-related disability, wherein the subject in need thereof comprises: (a) an increase in serum Neurofilament light chain (sNfL) levels over a baseline level of the subject in need thereof; or (b) an increase in sNfL levels over an otherwise comparable control subject. In aspects, the methods further comprise identifying the subject in need thereof.

Provided are methods of reducing neuromyelitis optica spectrum disorder (NMOSD)-related disability in a subject diagnosed with NMOSD, the methods comprising administering a composition that comprises Inebilizumab or a derivative thereof to the subject diagnosed with NMODS, wherein the subject diagnosed with NMODS comprises: (a) an increase in serum Neurofilament light chain (sNfL) level over a baseline level of the subject diagnosed with NMODS; or (b) an increase in sNfL level over an otherwise comparable control subject.

Provided are methods of treating neuromyelitis optica spectrum disorder (NMOSD) in a subject in need thereof, the methods comprising: (a) identifying a subject in need thereof at increased risk for NMOSD-related disability as determined by: (i) an increased serum Neurofilament light chain (sNfL) level over a baseline level of the subject in need thereof; or (ii) an increased sNfL level over an otherwise comparable control subject; and (b) administering a composition that comprises Inebilizumab or a derivative thereof to the subject identified in (a), thereby treating the NMODS.

Provided are methods of treating neuromyelitis optica spectrum disorder (NMOSD) in a subject in need thereof, the methods comprising administering a composition that comprises Inebilizumab or a derivative thereof to the subject in need thereof, thereby treating the NMODS, wherein the subject in need thereof comprises: an increased serum Neurofilament light chain (sNfL) level over a baseline level of the subject in need thereof; or (b) an increased sNfL level over an otherwise comparable control subject. In aspects, the subject in need thereof comprises about 1.0, about 1.1, about 1.2, about 1.3, about 1.4, about 1.5, about 1.6, about 1.7, about 1.8, about 1.9, about 2.0 or greater fold change in serum Nfl over a baseline level. In aspects, the subject in need thereof has a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater.

Provided are methods of treating a subject suspected of having neuromyelitis optica spectrum disorder (NMOSD), the methods comprising administering a composition that comprises Inebilizumab or a derivative thereof to the subject, wherein the subject comprises one or more NMODS-related symptoms and at least one of: (a) an increase in serum Neurofilament light chain (sNfL) level over a baseline level of the subject; or (b) an increase in sNfL level over an otherwise comparable control subject.

Provided are methods of treating a subject suspected of having neuromyelitis optica spectrum disorder (NMOSD), the methods comprising: (a) identifying a subject as having one or more NMOSD-related symptoms; (b) determining if the subject identified in (a) is at increased risk for NMOSD-related disability as determined by (i) an increase in serum Neurofilament light chain (sNfL) level over a baseline level of the subject identified in (a); or (ii) an increase in sNfL levels over an otherwise comparable control subject; and (c) administering a composition that comprises Inebilizumab or a derivative thereof to the subject determined to be at increased risk for NMOSD-related disability from (b).

Provided are methods of treating neuromyelitis optica spectrum disorder (NMOSD) in a subject in need thereof, the methods comprising administering a therapeutic in an amount effective to treat the NMODS in the subject in need thereof, wherein the subject in need thereof comprises: (a) an increase in serum Neurofilament light chain (sNfL) level over a baseline level of the subject in need thereof; or (b) an increase in sNfL level over an otherwise comparable control subject.

Provided are methods of treating a subject suspected of having neuromyelitis optica spectrum disorder (NMOSD), the methods comprising administering a therapeutic to the subject suspected of having NMOSD, wherein the subject suspected of having NMOSD comprises one or more NMODS-related symptoms and at least one of: (a) an increase in serum Neurofilament light chain (sNfL) level over a baseline level of the subject suspected of having NMOSD; or (b) an increase in sNfL level over an otherwise comparable control subject. In aspects, the therapeutic comprises one or more of Eculizumab, Satralizumab, Ublituximab, Ravulizumab, Rituximab, Azathioprine, Mycophenolate Mofetil, or a low dose corticosteroid.

The present disclosure provides methods of reducing NMOSD-related damage in a subject at increased risk therefor. In the methods, a subject is identified as at increased risk for NMOSD-related damage. The subject is identified as at increased risk for NMOSD-related damage if the subject comprises a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater. VIB551 is intravenously administered to the at increased risk subject to reduce the NMOSD-related damage.

In aspects, the subject is identified as at increased risk if the subject comprises a sGFAP concentration of about 170 pg/mL or greater. In certain aspects, the VIB551 is administered intravenously at a dose of 300 mg every 6 months. In aspects, reducing NMOSD-related damage comprises reducing number of NMOSD-related attacks in the at increased risk subject relative to a baseline number of NMOSD-related attacks in the at increased risk subject, wherein the baseline number of attacks are determined over a first time period preceding the administering VIB551, wherein the number of attacks reduced by the administering VIB551 are determined over a second time period following the administering VIB551, and wherein the first and the second time period are of equal length. In one aspect, the first and the second time period are at least one year. In aspects, reducing the NMOSD-related damage comprises reducing likelihood of NMOSD-related attacks in the at increased risk subject graded major in severity. In certain aspects, reducing the NMOSD-related damage comprises preventing NMOSD-related attacks in the at increased risk subject graded major in severity. In aspects, reducing the NMOSD-related damage comprises reducing number of magnetic resonance imaging (MRI) lesions or reducing rate of increase in new MRI lesions in the at increased risk subject. In one aspect, the reducing the NMOSD-related damage comprises reducing rate of worsening of expanded disability status scale (EDSS) score, or improving EDSS score, of the at increased risk subject.

The disclosure also provides methods of preventing or reducing the likelihood of NMOSD relapse in a subject diagnosed with NMOSD. In the methods, a subject is identified as a subject for preventing NMOSD relapse. The subject is identified as a subject for preventing NMOSD relapse if the subject comprises a sGFAP concentration of less than about 165 pg/mL, about 166 pg/mL, about 167 pg/mL, about 168 pg/mL, about 169 pg/mL, about 170 pg/mL, about 171 pg/mL, about 172 pg/mL, about 173 pg/mL, about 174 pg/mL, about 175 pg/mL, about 176 pg/mL, or about 181 pg/mL. VIB551 is administered to the subject identified as a subject to prevent or reduce the likelihood of NMOSD relapse.

In aspects, the subject is identified as a subject if the subject comprises a sGFAP concentration of less than about 170 pg/mL. In aspects, the VIB551 is administered intravenously at a dose of 300 mg every 6 months. In aspects, preventing comprises a time period of at least 1 year. In aspects, administering decreases sGFAP concentration in the subject relative to baseline sGFAP concentration or as compared to sGFAP in a control subject, and wherein the preventing comprises a time period of at least 2 years. In certain aspects, preventing results in a reduction in MRI lesions in the subject. In aspects, preventing results in an improvement in EDSS score in the subject.

The disclosure further provides for methods of suppressing a NMOSD-related attack in a subject diagnosed with NMOSD. In the methods, the subject is identified as at-risk for an NMOSD-related attack. The subject is identified as at-risk for an NMOSD-related attack if the subject's sGFAP concentration has increased relative to baseline sGFAP concentration of the subject or as compared to a control subject. In aspects, a therapeutic is administered to the at-risk subject at most one week following the identification to suppress the NMOSD-related attack.

In aspects, the increase in sGFAP concentration comprises an at least 10-fold increase; and wherein the at-risk subject is not undergoing a treatment for NMOSD that comprises VIB551. In aspects, the increase in sGFAP concentration comprises an at least 20-fold increase; and wherein the at-risk subject is not undergoing a treatment for NMOSD that comprises VIB551. In aspects, the increase in sGFAP concentration comprises an increase of 50% to 150%; and wherein the at-risk subject is undergoing treatment for NMOSD, wherein the treatment comprises VIB551. In aspects, the therapy comprises one or more of steroids, plasmapheresis, immunoadsorption or a complement inhibitor. In aspects, the treatment comprises one or more of Eculizumab, Satralizumab, Ublituximab, Ravulizumab, Rituximab, azathioprine, mycophenolate mofetil or low dose corticosteroids. In certain aspects, the administering is performed at most 24 hours following the identifying.

In aspects, suppressing the NMOSD-related attack comprises reducing likelihood of or preventing the NMOSD-related attack. In aspects, suppressing the NMOSD-related attack comprises reducing likelihood of or preventing the NMOSD-related attack from being graded as major in severity. In one aspect, suppressing the NMOSD-related attack comprises a recovery from the NMOSD-related attack that is graded as a major recovery. In aspects, suppressing the NMOSD-related attack results in a prevention of new MRI lesions in the at-risk subject. In certain aspects, suppressing the NMOSD-related attack results in a reduction in NMOSD-related disability in the at-risk subject. In aspects, the reduction in NMOSD-related disability is a reduction in worsening of the at-risk subject's EDSS score. In aspects, the therapy comprises VIB551.

The disclosure also provides for methods of treating NMOSD in a subject. In the methods, a therapeutically effective amount of a B cell depleting therapy is administered to the subject when the subject has a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater. In aspects, the subject has a sGFAP concentration of about 170 pg/mL to 171 pg/mL. In one aspect, the B cell depleting therapy is VIB551 and the therapeutically effective amount comprises a dose of 300 mg.

The disclosure also provides methods of reducing NMOSD-related disability in a subject diagnosed with NMOSD, the methods comprising: identifying a subject as at increased risk for NMOSD-related disability, wherein the subject is identified as at increased risk if the subject has an increase in serum Neurofilament light chain (NfL) levels over a baseline level; and administering VIB551 to the subject. The disclosure also provides methods of reducing NMOSD-related disability in a subject diagnosed with NMOSD, the methods comprising administering VIB551 to a subject with an increase in serum Nfl levels over a baseline level of the subject or as compared to serum Nfl levels of a control subject.

The disclosure also provides methods of treating NMOSD in a subject, the methods comprising: identifying a subject as at increased risk for NMOSD-related disability, wherein the subject is identified as at increased risk if the subject has an increase in serum Neurofilament light chain (NfL) levels over a baseline level; and administering VIB551 to the subject. The disclosure also provides methods of treating NMOSD in a subject, the methods comprising administering VIB551 to a subject with an increase in serum Nfl levels over a baseline level.

In aspects, the subject has an about 1.0, about 1.1, about 1.2, about 1.3, about 1.4, about 1.5, about 1.6, about 1.7, about 1.8, about 1.9, about 2.0 or greater fold change in serum Nfl over baseline levels. In aspects, the subject has a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater.

The disclosure also provides methods of treating a subject suspected of having NMOSD, the methods comprising administering VIB551 to a subject with an increase in serum Nfl levels over a baseline level. In aspects the subject also comprises one or more NMOSD-related symptoms.

The disclosure also provides methods of treating a subject suspected of having NMOSD, the methods comprising: identifying a subject as having one or more NMOSD-related symptoms; identifying a subject as at increased risk for NMOSD-related disability, wherein the subject is identified as at increased risk if the subject has an increase in serum Neurofilament light chain (NfL) levels over a baseline level; and administering VIB551 to the subject.

The disclosure also provides methods of treating NMOSD in a subject, the methods comprising a administering a therapeutic to a subject with an increase in serum Nfl levels over a baseline level. The disclosure also provides methods of treating a subject suspected of having NMOSD, the methods comprising administering a therapeutic to a subject with an increase in serum Nfl levels over a baseline level and one or more NMOSD-related symptoms. In aspects, the therapeutic comprises one or more of Eculizumab, Satralizumab. Ublituximab, Ravulizumab, Rituximab, azathioprine, mycophenolate mofetil or low dose corticosteroids.

These and other aspects are described below.

### BRIEF DESCRIPTION OF THE DRAWINGS

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1 is an exemplary Consolidated Standards of Reporting Trials (CONSORT) flow diagram for the N-MOmentum study participants. ^{a}These subjects did not meet eligibility criteria due to one or more of: baseline laboratory values (n=68); neuromyelitis optica spectrum disorder diagnosis or minimum disease criteria (n=57); positive tuberculosis test (n=32); hepatitis (n=16); contraindicated health condition (n=13); not stable after a recent attack (n=10); receipt of contraindicated medications in the previous 6 months (n=9); unable or unwilling to participate (n=6); unable to undergo magnetic resonance imaging scan (n=3); Expanded Disability Status Scale score (n=1); RCP, randomized controlled period. ITT, intention-to-treat.

FIGS. 2A-2B. FIG. 2A provides a graph showing baseline sGFAP concentration in each of the NMOSD, relapsing-remitting multiple sclerosis (RRMS), and Healthy Donor (HD) cohorts; dashed line represents 2 standard deviations from the HD mean (170 pg/mL); box and whiskers represent sample quartiles; statistical significance of differences in sGFAP concentration between groups was assessed using the Mann-Whitney U test. FIG. 2B provides baseline sGFAP for healthy donors, RRMS subjects and NMOSD subjects by serostatus according to whether they were aquaporin 4-immunoglobulin G seropositive (AQP4+), aquaporin 4-immunoglobulin G seronegative (AQP4−), myelin oligodendrocyte glycoprotein-immunoglobulin G seropositive (MOG+), or both AQP4−/MOG− (double negative; “DN”). Box and whiskers represent sample quartiles.

FIGS. 3A-3F. FIG. 3A provides baseline sGFAP for healthy donors according to age. FIG. 3B provides baseline sGFAP for subjects with NMODS according to age. FIG. 3C provides baseline sGFAP for healthy donors according to gender. FIG. 3D provides baseline sGFAP for subjects with NMODS according to gender. FIG. 3E provides baseline sGFAP for healthy donors according to ethnicity. FIG. 3F provides baseline sGFAP for healthy donors according to ethnicity.

FIGS. 4A-4E. FIG. 4A provides a graph showing Kaplan-Meier plots of time until first NMOSD attack, i.e., attack-free survival, for all NMOSD trial participants according to whether their baseline sGFAP was elevated. FIG. 4B shows a Kaplan-Meier plot of time until first NMOSD attack, i.e., attack-free survival, during the RCP for NMOSD trial participants with (Q 170 pg/mL) or without (<170 pg/mL) an elevated baseline sGFAP concentration who received placebo. FIG. 4C shows a Kaplan-Meier plot of time until first NMOSD attack, i.e., attack-free survival, during the RCP for NMOSD trial participants with (≥170 pg/mL) or without (<170 pg/mL) an elevated baseline sGFAP concentration who were administered Inebilizumab. For both FIG. 4B and FIG. 4C statistical significance of difference in time until first adjudicated attack between groups was assessed using Wald's test; *P<0.05; ***P<0.001. FIG. 4D shows a Kaplan-Meier plot of time until first NMOSD attack, i.e., attack-free survival, in NMOSD trial participants who received placebo or Inebilizumab with ≥170 pg/mL elevated baseline sGFAP concentration. FIG. 4E shows a Kaplan-Meier plot of time until first NMOSD attack, i.e., attack-free survival, in NMOSD trial participants who received placebo or Inebilizumab with <170 pg/mL elevated baseline sGFAP concentration. For both FIG. 4D and FIG. 4E statistical significance of difference in time until first adjudicated attack between groups was assessed using Wald's test; HR, hazard ratio; Inebilizumab=VIB551.

FIGS. 5A-5D. FIG. 5A provides a graph showing a profile plot of sGFAP concentration measurements taken at baseline and at visits leading to an adjudicated NMOSD attack for each of 37 NMOSD trial participants who had both experienced an adjudicated attack and provided sGFAP measurements; overall, 29/37 (78%) samples were above the healthy donor range of 170 pg/mL. FIG. 5B provides baseline sGFAP levels and sGFAP levels of NMOSD trial participants who experienced an adjudicated attack in each of the four weeks leading up to, and at the approximate time of, an NMOSD-related attack. FIG. 5C provides sGFAP concentration (pg/mL) during an adjudicated attack in trial participants who were AQP4+ and placebo-treated, AQP4+ and VIB551-treated, AQP4− MOG+ and VIB551-treated, and DN and VIB551-treated. No AQP4− placebo-treated subjects experienced an attack during the RCP. FIG. 5D provides the fold change in baseline sGFAP during an adjudicated attack in trial participants who were AQP4+ and placebo-treated, AQP4+ and VIB551-treated, AQP4− MOG+ and VIB551-treated, and DN and VIB551-treated.

FIG. 6A shows sGFAP concentration within 1 week of (before or after) an adjudicated NMOSD attack by attack severity. Attack severity was measured by the opticospinal impairment scale. FIG. 6B shows sGFAP concentration within 1 week of (before or after) an adjudicated NMOSD attack by domain involvement. Of the attacks across multiple domains, 4 were minor myelitis attacks, and 1 sample from myelitis major attack group displayed sGFAP within the healthy donor range. Box and whiskers represent sample quantiles. Statistical significance between groups was assessed using Mann-Whitney U test. Dotted line in each graph represents the border of the healthy donor range of 170 pg/mL sGFAP; **P<0.01; ***P<0.001; ns, not significant.

FIGS. 7A-7D. sGFAP concentration predicts attack severity in both placebo and Inebilizumab treated participants. FIG. 7A provides a boxplot of sGFAP concentrations for placebo-treated trial participants within 1 week of an adjudicated NMOSD attack, split by attack severity. FIG. 7B provides a boxplot of sGFAP concentrations for placebo-treated trial participants within 1 week of an adjudicated NMOSD attack, split by domain involvement. FIG. 7C provides a boxplot of sGFAP concentrations for Inebilizumab-treated trial participants within 1 week of an adjudicated NMOSD attack, split by attack severity. FIG. 7D provides a boxplot of sGFAP concentrations for Inebilizumab-treated trial participants within 1 week of an adjudicated NMOSD attack, split by domain involvement. Attack severity was measured by the opticospinal impairment scale; Box and whiskers represent sample quantiles; Significance between groups assessed using Mann-Whitney U test (n.s.—not significant, *p<0.05, **p<0.01, ***p<0.001); Inebilizumab=VIB551.

FIGS. 8A-8D. FIG. 8A shows sGFAP concentration during adjudicated NMOSD attacks in placebo-treated participants; statistical significance of increases from baseline was assessed using the Wilcoxon signed-rank test. FIG. 8B shows sGFAP concentration during adjudicated NMOSD attacks in Inebilizumab-treated participants; statistical significance of increases from baseline was assessed using the Wilcoxon signed-rank test. FIG. 8C shows the change in sGFAP concentration from baseline in both placebo- and Inebilizumab-treated participants who did not experience an adjudicated NMOSD attack, error bars represent interquartile range, statistical significance of percentage change from baseline was assessed using the Mann-Whitney U test; 19/117 Inebilizumab samples (16%) and 9/26 placebo samples (35%) were outside healthy donor range (Fisher's test; P=0.052). FIG. 8D provides a bar graph to show the proportion of samples from placebo- and, separately, Inebilizumab-treated subjects with elevated sGFAP concentrations (>171 pg/mL) by the end of the RCP.

FIGS. 9A-9E demonstrate that in NMOSD subjects who do not experience an adjudicated attack, increased sGFAP signaled increased NMOSD-related disease activity. FIG. 9A provides a profile plot of longitudinal fold change from baseline in sGFAP in subjects with NMOSD who experienced adjudicated attacks (light gray), those who did not experience adjudicated attacks but displayed an increase greater than twofold from baseline (dark gray), and in those who neither experienced attacks nor displayed increases greater than twofold from baseline (mid gray) in sGFAP during the RCP. FIG. 9B provides boxplots displaying sGFAP concentrations observed in 10 healthy donor (HD) subjects across three blood draws. FIG. 9C provides the proportion of new spinal cord T2 lesions observed in subjects who did not experience adjudication committee (AC)-adjudicated attacks, but either did or did not display a greater than twofold (FC) increase in sGFAP during the RCP. FIG. 9D provides the proportion of subjects with new Gd positive T1 lesions observed in subjects who did not experience adjudication committee (AC)-adjudicated attacks, but either did or did not display a greater than twofold (FC) increase in sGFAP during the RCP. FIG. 9E provides a graph showing proportion of participants with an increase greater than two-fold in sGFAP from baseline. Statistical significance in the between-group difference was assessed using the Cochran-Armitage test.

FIG. 10A provides VIB551's VH (SEQ ID NO:1) amino acid sequence and FIG. 10B provides VIB551's VL (SEQ ID NO:2) amino acid sequence. The amino acid sequence of each of VIB551's VH CDR1 (SEQ ID NO:3), VH CDR2 (SEQ ID NO:4), VH CDR3 (SEQ ID NO:5), VL CDR1 (SEQ ID NO:6), VL CDR2 (SEQ ID NO:7) and VL CDR3 (SEQ ID NO:8) is separately indicated within its respective VH and VL amino acid sequence.

FIG. 11 shows the disposition, demographics and baseline characteristics of the AQP4-IgG seropositive vs. AQP4-IgG seronegative subgroup of the N-MOmentum study. 1 subject (AQP4-IgG seronegative) was randomized to Inebilizumab but did not receive treatment. Abbreviations: AQP4, aquaporin-4; EDSS, (Kurtzke) Expanded Disability Status Scale; IgG, immunoglobulin G; IgG1, immunoglobulin G1; MOG, myelin oligodendrocyte glycoprotein.

FIG. 12 shows the NMOSD attacks during the RCP for AQP4-IgG seronegative subjects. Abbreviations: AC, adjudication committee; AQP4, aquaporin-4; Gd+, Gadolinium-enhancing; IgG, immunoglobulin G; IgG1, immunoglobulin G1; MOG, myelin oligodendrocyte glycoprotein; NMO/NMOSD, neuromyelitis optical neuromyelitis optica spectrum disorder; ON, optic nerve; RCP, randomized-controlled period.

FIG. 13 shows the annualized attack rates during RCP (post hoc analysis) for AQP4-IgG seronegative subjects. Abbreviations: AQP4, aquaporin-4; CI, confidence interval; IgG, immunoglobulin G; MOG, myelin oligodendrocyte glycoprotein; RCP, randomized-controlled period.

FIG. 14 shows the annualized attack rates during OLE for AQP4-IgG seronegative subjects.

FIGS. 15A-15D. Demonstrate that biomarkers of neuronal injury were elevated in subjects with NMOSD. FIG. 15A shows increased levels of sGFAP in NMOSD subjects as compared to HC and RRMS. FIG. 15B shows increased levels of sNfl in NMOSD subjects as compared to HC and RRMS. FIG. 15C shows increased levels of sUCHL1 in NMOSD subjects as compared to HC and RRMS. FIG. 15D shows increased levels of sTau in NMOSD subjects as compared to HC and RRMS. Abbreviations: HC, healthy control; NMOSD, neuromyelitis optica spectrum disorder; sGFAP, serum glial fibrillary acidic protein; sNfL, soluble neurofilament light chain; sUCH-L1, soluble ubiquitin carboxyl-terminal hydrolase L1.

FIGS. 16A-16D. Demonstrate that the NMOSD attacks increased biomarker levels. FIG. 16A shows median Fc change from baseline in sGFAP in placebo-treated or Inebilizumab-treated subjects. FIG. 16B shows median Fc change from baseline in sNfl in placebo-treated or Inebilizumab-treated subjects. FIG. 16C shows median Fc change from baseline in sTau in placebo-treated or Inebilizumab-treated subjects. FIG. 16D shows median Fc change from baseline in sUCHL1 in placebo-treated or Inebilizumab-treated subjects. Abbreviations: FC, fold change; NMOSD, neuromyelitis optica spectrum disorder; sGFAP, serum glial fibrillary acidic protein; sNfL, soluble neurofilament light chain; sUCH-L1, soluble ubiquitin carboxyl-terminal hydrolase L1.

FIGS. 17A-17D. Demonstrate that baseline elevations in biomarkers were significantly correlated with increased attack risk. FIG. 17A shows percent of subjects that are attack free as a function of time and sGFAP status. FIG. 17B shows percent of subjects that are attack free as a function of time and sNfl status. FIG. 17C shows percent of subjects that are attack free as a function of time and sTau status. FIG. 17D shows percent of subjects that are attack free as a function of time and sUCH-L1 status. Abbreviations: HR, hazard ratio; NMOSD, neuromyelitis optica spectrum disorder; sGFAP, serum glial fibrillary acidic protein; sNfL, soluble neurofilament light chain; sUCH-L1, soluble ubiquitin carboxyl-terminal hydrolase L1.

FIG. 18 Provides a sGFAP baseline-controlled regression analysis demonstrated that biomarkers other than sGFAP were not independently associated with attack risk. Abbreviations: NMOSD, neuromyelitis optica spectrum disorder; sGFAP, serum glial fibrillary acidic protein; sNfL, soluble neurofilament light chain; sUCH-L1, soluble ubiquitin carboxyl-terminal hydrolase L1

FIG. 19 Demonstrates that sNfL at attack is strongest correlate of EDSS change at attack follow-up.

### DETAILED DESCRIPTION

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

Any ranges listed herein are intended to be inclusive of endpoints. For example, a range of 2-4 includes 2 and 4 and values between.

As used herein, the singular forms “a.” “an,” and “the: include plural referents unless the content clearly dictates otherwise.

The term “about” or “approximately” when immediately preceding a numerical value means a range plus or minus 10% of that value. For example, “about 50” can mean 45 to 55, “about 25,000” can mean 22,500 to 27,500, etc., unless the context of the disclosure indicates otherwise, or is inconsistent with such an interpretation. In the context of a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

### Overview

Provided herein are compositions and methods of use thereof for the treatment or prevention of Neuromyelitis Optica Spectrum Disorder (NMOSD) and associated disabilities and symptoms. Described herein are also methods of reducing NMOSD-related damage in a subject at increased risk therefor, preventing NMOSD relapse in a subject diagnosed with NMOSD, suppressing an NMOSD-related attack in a subject diagnosed with NMOSD, and treating NMOSD in a subject. In each of the methods, serum glial fibrillary acidic protein (sGFAP) concentration can be useful in identifying: a subject in need thereof, a subject in need of treatment for NMOSD, or a subject in need of an adjustment in treatment for NMOSD. In any methods disclosed herein, increases in serum Neurofilament light (Nfl) levels over baseline levels may be used to identify subjects at increased risk for NMOSD-related disability. In aspects, the subject is AQP4-IgG seronegative. In aspects, the subject is AQP4-IgG seropositive.

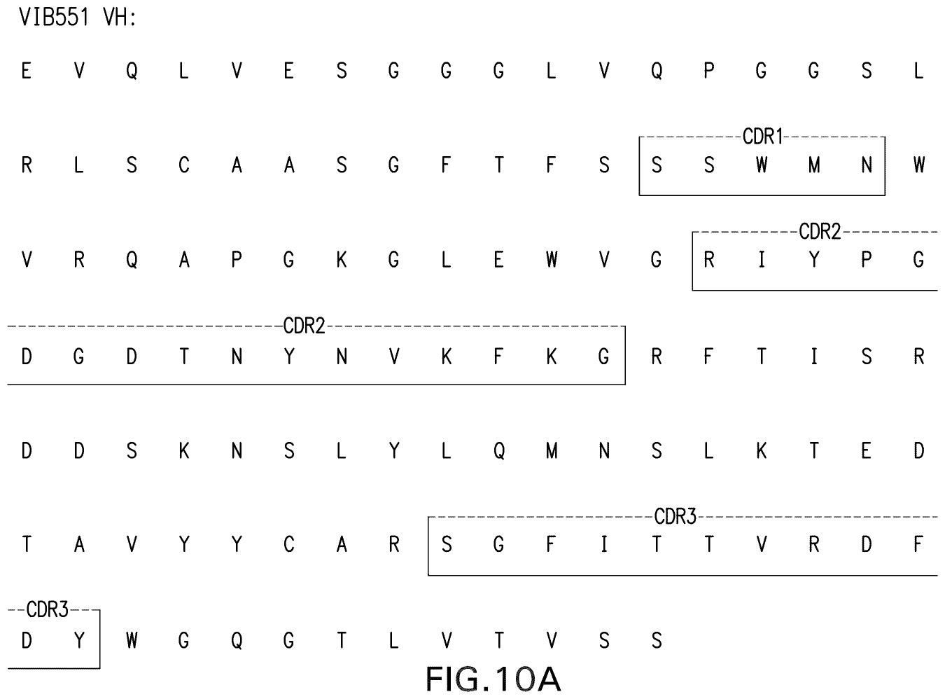

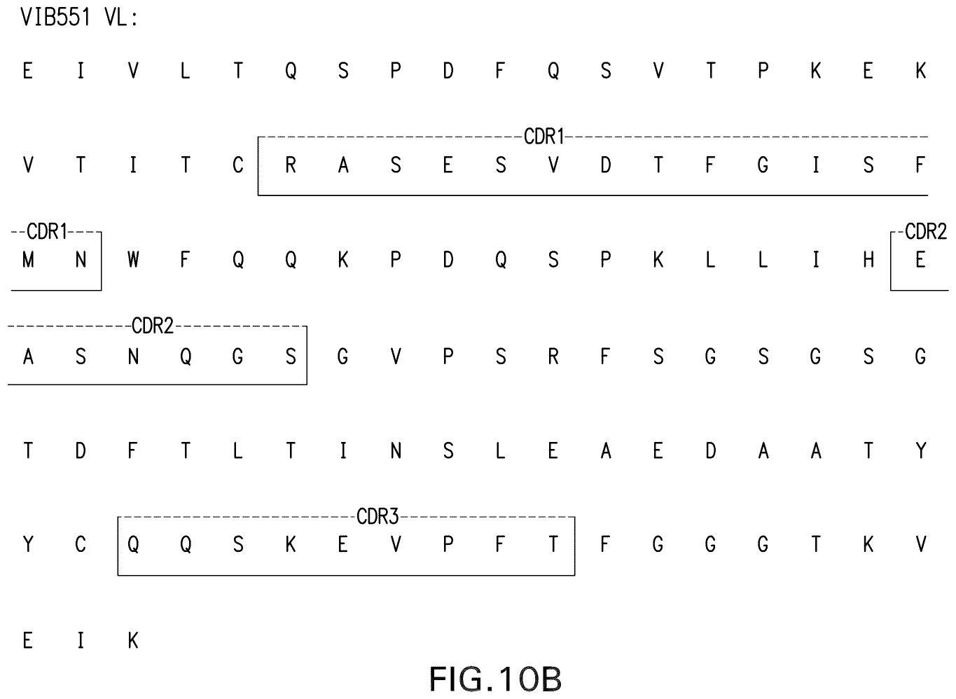

Compositions that comprise Inebilizumab or a derivative thereof may be administered in any of the methods, including but not limited to, methods of treating NMOSD, described herein. For example, if the subject is identified as at increased risk for NMOSD-related damage, or if the subject is identified as a subject for preventing or reducing the likelihood of NMOSD relapse, Inebilizumab or a derivative thereof may be administered to the subject, and the administration of Inebilizumab or the derivative thereof may be intravenously at a dose of 300 mg every six months. As used herein, VIB551 is a humanized antibody with a VH amino acid sequence and a VL amino acid sequence as shown in FIG. 10A and FIG. 10B VIB551 may also be referred to as MEDI551, Inebilizumab or UPLIZNA™. VIB551 and methods of making thereof are described in International PCT Patent Application PCT/US2007/077916, published as WO 2008/031056, which is hereby incorporated by reference (PCT/US2007/077916 refers to VIB551 as “16C4”). In aspects, VIB551 (also referred to HZN551, MEDI551, UPLIZNA™ or Inebulizumab; disclosed in U.S. application Ser. No. 11/852,106 and Int'l Appl. No. PCT/US20/29613, which are incorporated herein by reference in their entireties) is administered in any of the methods disclosed herein. A derivative of VIB551 includes but is not limited to an antibody with the VH amino acid sequence and the VL amino acid sequence as shown in FIG. 10A and FIG. 10B, but for one or more substitutions in amino acid residues that do not alter the function of VIB551. In aspects, a VIB551 derivative is an antibody with the VH amino acid sequence and the VL amino acid sequence as shown in FIG. 10A or FIG. 10B, with 1, 2, 3, 4, or 5 amino acid residue substitutions and/or deletions. In aspects, a derivative of Inebilizumab includes the same CDR amino acid sequences as the VH and the VL sequences as shown in FIG. 10A or FIG. 10B, but may have one or more amino acid substitutions in the framework regions of the VH and the VL sequences shown in FIG. 10A or FIG. 10B. In aspects, Inebilizumab, a portion thereof, or a derivative thereof comprises at least about or at most about 50%, 55%, 60%, 65%. 70%, 75%. 80%, 85%. 90%, 95%, or up to about 99% identity with any one of SEQ ID NO: 1-SEQ ID NO: 10. In aspects, a VH of Inebilizumab corresponds to SEQ ID NO:1. In aspects, a VL of Inebilizumab corresponds to SEQ ID NO:2. In aspects, the amino acid sequence of each of Inebilizumab's VH CDR1 (SEQ ID NO:3), VH CDR2 (SEQ ID NO:4), VH CDR3 (SEQ ID NO:5), VL CDR1 (SEQ ID NO:6), VL CDR2 (SEQ ID NO:7) and VL CDR3 (SEQ ID NO:8) correspond to each of the aforementioned sequences. In aspects, Inebilizumab may comprise a heavy chain comprising the amino acid of SEQ ID NO:9 and a light chain comprising the amino acid of SEQ ID NO:10. In aspects. Inebilizumab may have the heavy chain amino acid sequence of SEQ ID NO:9 and the light chain amino acid sequence of SEQ ID NO:10 but for one or more changes in amino acid residues that do not alter the function of Inebilizumab. The number of amino acid changes may be 1 amino acid residue change, 2 amino acid residue changes, 3 amino acid residue changes, 4 amino acid residue changes, or 5 amino acid residue changes. In aspects, a sequence of SEQ ID NO: 1-SEQ ID NO: 10 comprises an amino acid residue insertion, deletion, or modification of 0-10, 0-2, 0-5, 0-3, or 1-5 residues. In aspects, a sequence of SEQ ID NO: 1-SEQ ID NO: 10 comprises an amino acid residue insertion, deletion, or modification of 0, 1, 2, 3, 4, 5, 6, 7, 8, 9, or up to 10 residues.

The administration of a compositions that comprises Inebilizumab or a derivative thereof in the methods described herein may be every 6 months or approximately 6 months. For example, the administration of Inebilizumab or the derivative thereof may be every 6 months, every 180 days, between about every 170 and about every 190 days, between about every 175 and about every 185 days, between about every 175 and about every 190 days, or between about every 170 and about every 185 days. The administration of Inebilizumab or the derivative thereof may be about every 26 weeks, about every 25 weeks, about every 27 weeks, between about every 25 and about ever 27 weeks, between about every 25 and about every 26 weeks, or between about every 26 and about every 27 weeks.

Prior to administering Inebilizumab or the derivative thereof every six months in the methods described herein, an initial Inebilizumab or derivative of Inebilizumab dose may be administered to the subject. If an initial dose is administered, the initial dose may be administered approximately 2 weeks before the approximately every 6-month dosing. The administering of the initial dose approximately 2 weeks before the every approximately 6-month dosing may be the administering of the initial dose 12 days, 13 days, 14 days, 15 days, or 16 days before the approximately every 6 months dosing. The initial dose may or may not be co-administered with oral corticosteroids.

The dose of Inebilizumab or the derivative thereof administered intravenously in the methods described herein may be 300 mg or approximately 300 mg. An approximately 300 mg dose may be a dose of about 250 mg to about 350 mg, it may be a dose of about 275 mg to about 325 mg, it may be a dose of about 290 mg to about 310 mg, it may be a dose of about 205 mg to about 305 mg, or it may be a dose of 275 mg, 280 mg, 285 mg, 290 mg, 295 mg, 300 mg, 305 mg, 310 mg, 315 mg, 320 mg, or 325 mg.

It is further contemplated, and will also be understood, that in methods described herein, e.g., of reducing NMOSD-related damage in a subject at increased risk therefor or of preventing NMOSD relapse in a subject diagnosed with NMOSD, an alternative therapeutically acceptable NMOSD agent may be administered to the subject in place of Inebilizumab or a derivative thereof, e.g., administered to the subject identified as at increased risk for NMOSD-related damage or identified as a subject for preventing or reducing the likelihood of NMOSD relapse.

An alternative therapeutically acceptable NMOSD agent may be a B cell depleting therapy other than Inebilizumab, such as CD19 antibody, e.g., MOR00208 (also referred to as Xmab 5574 or tafasitamab; disclosed in U.S. Patent Application No. 20170137516), an anti-CD20 antibody, e.g., Rituximab (antibody C2B8 in WO94/11026), Ocrelizumab (also referred to as Ocrevus® or PRO70769; disclosed in Vugmeyster, Y., et al., J. Immunother. 28(2005):212-219, WO 2004/056312 and WO 2006/084264), Ofatumumab (also referred to as HuMax-CD20 or Azerra®; disclosed as antibody 2F2 in WO04/35607) or Obinutuzumab (also referred to as Gazvva®; disclosed in WO2017/148880); an anti-CD22 antibody, e.g., Epratuzumab (antibody hLL2 in U.S. Pat. No. 5,789,554); or a BLyS inhibitor, e.g., Belimumab (also referred to as Lymphostat-B; disclosed in WO 02/02641), BR3-Fc (disclosed in WO 05/00351), AMG-623 (also referred to as blisibimod; PubChem SID: 163312341), or Atacicept (U.S. Patent Application Publication No. 20060034852).

Further, an alternative acceptable NMOSD therapeutic agent may be an NMOSD therapeutic agent that blocks a complement component, such as complement component C5, e.g., Eculizumab (also referred to as Soliris®; U.S. Pat. No. 6,355,245). If the alternative acceptable NMOSD therapeutic agent is Eculizumab, then Eculizumab may be administered to the subject at a dose of approximately 900 mg once per week for four weeks, followed by a dose of approximately 1200 mg one week following the fourth 900 mg dose, further followed by a 900 mg dose every two weeks following the first 900 mg dose.

In addition, an alternative acceptable NMOSD therapeutic agent may be an NMOSD therapeutic agent that binds to and blocks interleukin (IL)-6 receptor (R). If the alternative acceptable NMOSD therapeutic agent binds to and blocks IL-6R, the therapeutic agent may be Satralizumab (also referred to as SA-237; disclosed in U.S. Patent Application Publication 2018/0148509). If the therapeutic agent is Satralizumab, then Satralizumab may be administered to the subject subcutaneously at a dose of approximately 120 mg once every other week for an initial three doses, and then every fourth week after the administration of the initial three doses.

Other alternative acceptable NMOSD therapeutic agents may include azathioprine, prednisone, azathioprine in combination with prednisone, mycophenolate, methotrexate, or methotrexate in combination with corticosteroids/cyclophosphamide, or others known in the art.

In methods provided herein, NMOSD-related damage is reduced in a subject who is at increased risk for NMOSD-related damage. The subject may be identified as at increased risk for NMOSD-related damage if the subject comprises a sGFAP concentration of about 160 pg/mL, 160 pg/mL, about 165 pg/mL, 165 pg/mL, about 166 pg/mL, 166 pg/mL, about 167 pg/mL, 167, pg/mL, about 168 pg/mL, 168 pg/mL, about 169 pg/mL, 169 pg/mL, about 170 pg/mL, 170 pg/mL, about 171 pg/mL, 171 pg/mL, about 172 pg/mL, 172 pg/mL, about 173 pg/mL, 173 pg/mL, about 174 pg/mL, 174 pg/mL, about 175 pg/mL, 175 pg/mL, about 176 pg/mL, 176 pg/mL, or greater. Furthermore, the subject may be identified as at increased risk for NMOSD-related damage if the subject comprises a sGFAP concentration of between about 166 pg/mL and about 176 pg/mL, between about 167 pg/mL and about 175 pg/mL, between about 168 pg/mL and about 174 pg/mL, or between about 169 pg/mL and about 173 pg/mL or greater. Further, the subject may be identified as at increased risk for NMOSD-related damage if the subject comprises an sGFAP concentration that is approximately 2 standard deviations above or 3 standard deviations above a healthy donor's mean sGFAP concentration or greater. In aspects, the subject may comprise a sGFAP concentration of about, or approximately, 170 pg/mL, e.g., a sGFAP concentration of 170 pg/mL, or greater. It will be understood that a measurement of sGFAP concentration, e.g., of 170 pg/mL, may be an sGFAP concentration that takes into account any deviation or variation, e.g., from 170 pg/mL, caused by a device employed to measure sGFAP concentration, e.g., device calibration, or by sample handling or processing leading up to measurement of sGFAP concentration. In aspects, the subject also has an increase in serum Neurofilament light chain (Nfl) levels over baseline Nfl levels of the subject or as compared to a control subject. In aspects, the subject has a sGFAP concentration of about 160 pg/mL, 160 pg/mL, about 165 pg/mL, 165 pg/mL, about 166 pg/mL, 166 pg/mL, about 167 pg/mL, 167, pg/mL, about 168 pg/mL, 168 pg/mL, about 169 pg/mL, 169 pg/mL, about 170 pg/mL, 170 pg/mL, about 171 pg/mL, 171 pg/mL, about 172 pg/mL, 172 pg/mL, about 173 pg/mL, 173 pg/mL, about 174 pg/mL, 174 pg/mL, about 175 pg/mL, 175 pg/mL, about 176 pg/mL, 176 pg/mL, or greater and an increase in serum Nfl levels over baseline levels of a subject or as compared to a control subject.

In the methods of reducing NMOSD-related damage in the subject at increased risk therefor, the NMOSD-related damage reduced by the methods may be: a reduction in number of NMOSD-related attacks in the at increased risk subject, a reduction in severity of NMOSD-related attacks in the at increased risk subject, an improvement in recovery from NMOSD-related attacks in the at increased risk subject, a reduction in number of magnetic resonance imaging (MRI) lesions in the at increased risk subject, a reduction in rate of increase in new MRI lesions in the at increased subject, a reduction in rate of worsening of Expanded Disability Status Scale (EDSS) score in the at increased risk subject, an improvement in EDSS score in the at increased risk subject, a reduction NMOSD-related pain in the at increased risk subject, or a reduction in NMOSD-related disability in the at increased risk subject. In aspects, reducing the NMOSD-related damage in a subject in need thereof comprises: (a) reducing a number of magnetic resonance imaging (MRI) lesions; (b) reducing rate of increase in new MRI lesions; or (c) both (a) and (b). In aspects, the reducing the rate of increase can also refer to reducing appearance of new MRI lesions or reducing the rate of increase over a period of time. In aspects, the time can comprise a period of about 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 25 months, 26 months, 27 months, 28 months, 29 months, 30 months, 31 months, 32 months, 33 months, 34 months, 35 months, or 36 months.

If the NMOSD-related damage reduced in the subject at increased risk therefor is a reduction in number of NMOSD-related attacks, then the number of the subject's NMOSD-related attacks may be reduced relative the subject's baseline number of NMOSD-related attacks. The subject's baseline number of NMOSD-related attacks may be the number of NMOSD-related attacks experienced by the subject during a first time period that precedes the administering of VIB551 or a derivative thereof. The subject's number of NMOSD-related attacks that are decreased, relative to baseline, may be the number of attacks experienced by the subject during a second time period following the administering of a first dose of a composition that comprises Inebilizumab or a derivative thereof. The first and the second time period may or may not be of equal length. If the first and the second time period are of equal length, then the first and second time period may both be of a length in time of approximately 6 months, 6 months, approximately 12 months, 12 months, approximately 18 months, 18 months, approximately 24 months, 24 months, approximately 30 months, 30 months, approximately 36 months, 36 months, approximately 42 months, 42 months, approximately 48 months, 48 months, approximately 54 months, 54 months, approximately 60 months, 60 months, approximately 6 years. 6 years, approximately 7 years, 7 years, approximately 8 years, 8 years, approximately 9 years, 9 years, approximately 10 years, or 10 years. It will be understood that the first and the second time period need not be exactly the same length in time, i.e., need not be exactly the same number of days. Rather, it will be understood that the first and the second time period may be considered to be of equal length if the number of days of the first time period is greater or lesser than 10%. 8%. 6%, 4%, 2%, or 1% the number of days in the second time period.

The first time period, which precedes the administering of a compositions that comprises Inebilizumab or a derivative thereof and is the time period during which the baseline number of NMOSD-related attacks is determined, may end the day before the administering of Inebilizumab or the derivative thereof. Alternatively, the first time period may end at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days or at most 1 month before the administering of a compositions that comprises Inebilizumab or a derivative thereof.

The second time period, which follows the administering of a composition that comprises Inebilizumab or a derivative thereof and is the time period during which the number of NMOSD-related attacks may be reduced, may begin the day of the administering of a first dose of Inebilizumab or derivative thereof. Alternatively, the second time period may begin at most 2 days, at most 3 days, at most 4 days, at most 5 days, at most 6 days, at most 7 days, at most 8 days, at most 9 days, at most 10 days, at most 11 days, at most 12 days, at most 13 days or at most 14 days following the administering of the first dose of Inebilizumab or derivative thereof.

The number of NMOSD-related attacks in the subject at increased risk for NMOSD-related damage may be reduced during the second time period by at least 1, 1, at least 2, 2, at least 3, 3, at least 4, 4, at least 5, or 5 following the administering of Inebilizumab or the derivative thereof relative to baseline of the subject or as compared to a control subject.

In methods of reducing NMOSD-related damage in the subject at increased risk therefor, the reduction in NMOSD-related damage may be a reduction in likelihood that the subject suffers a major NMOSD-related attack, e.g. attack graded major in severity. If the at increased risk subject is at a reduced likelihood of suffering a major NMOSD-related attack, then the reduced likelihood may be a prevention of the subject suffering the major NMOSD-related attack. Alternatively, the reduced likelihood may be a decrease in risk that the subject will suffer a major NMOSD-related attack of between 25% and 100%, or between 50% and 100%, or between 75% and 100% or between 25% and 75%, or between 50% and 75%, or by at least 25%, at least 50%, or at least 75%.

The reduction in likelihood that the at increased risk subject suffers a major NMOSD-related attack, e.g. attack graded major in severity, may be demonstrated by a reduction in number of severe NMOSD-related attacks experienced by the subject in a time period following the administering of a first dose of Inebilizumab or derivative thereof, (e.g., a second time period), relative to a time period preceding the administering of Inebilizumab or derivative thereof, (e.g., a first time period), in which the first and the second time period are of equal length. If the first and the second time period are of equal length, the first and the second time period may be approximately 6 months, 6 months, approximately 12 months, 12 months, approximately 18 months, 18 months, approximately 24 months, 24 months, approximately 30 months, 30 months, approximately 36 months, 36 months, approximately 42 months, 42 months, approximately 48 months, 48 months, approximately 54 months, 54 months, approximately 60 months, 60 months, approximately 6 years, 6 years, approximately 7 years, 7 years, approximately 8 years, 8 years, approximately 9 years, 9 years, approximately 10 years, or 10 years. It will be understood that the first and the second time period need not be exactly the same length in time, i.e., need not be exactly the same number of days. Rather, it will be understood that the first and the second time period may be considered to be of equal length if the number of days of the first time period is greater or lesser than 10%, 8%, 6%, 4%, 2%, or 1% the number of days in the second time period.

The first time period, preceding the administering Inebilizumab or derivative thereof, may end the day before the administering of Inebilizumab or derivative thereof. Alternatively, the first time period may end at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days or at most 1 month before the administering of Inebilizumab or derivative thereof. The second time period, following the administering of a first dose of Inebilizumab or derivative thereof, and that is the time period over which the number of NMOSD-related attacks graded as severe may be reduced, may be a time period that begins the day of the administering of a first dose of Inebilizumab or derivative thereof. Alternatively, the second time period may begin at most 2 days, at most 3 days, at most 4 days, at most 5 days, at most 6 days, at most 7 days, at most 8 days, at most 9 days, at most 10 days, at most 11 days, at most 12 days, at most 13 days or at most 14 days following the administering of the first dose of Inebilizumab or derivative thereof.

An NMOSD-related attack graded major in severity, the likelihood of which is reduced in the at increased risk subject by the administering of Inebilizumab or derivative thereof, may be any NMOSD-related attack that requires intensive therapeutic intervention, interrupts usual activities of daily living, significantly affects the subject's clinical status, or requires in-subject hospitalization. An NMOSD-related attack graded as major in severity may be an NMOSD-related attack that, if it affects brain, results in an increase in the subject's brain domain subscale score of 2 or more points when compared to the subject's brain domain subscale score prior to the NMOSD-related attack. An NMOSD-related attack graded as major in severity may be an NMOSD-related attack that, if it affects any of the subject's optic nerve, spinal cord or brainstem, results in the affected domain's subscale score increasing by ≥3 points when compared the affected domain's subscale score prior the attack, wherein the affected domain's subscale score had been less than 2 prior to the NMOSD-related attack. An NMOSD-related attack graded as major in severity may be an NMOSD-related attack that, if it affects any of the subject's optic nerve, spinal cord or brainstem, results in the affected domain's subscale score increasing by ≥2 when compared the affected domain's subscale score prior the attack, wherein the affected domain's subscale score had been ≥2 prior to the NMOSD-related attack. Domain subscale scores may be determined according to the domain numerical assignments presented in Table 2.

The NMOSD-related attacks, reduced in number or reduced in likelihood of being graded as severe attacks, in the methods may be attacks characterized by the appearance of a new NMOSD symptom or the worsening of an existing NMOSD symptom. The new or worsening existing symptom which characterizes the NMOSD-related attack may be an eye symptom, a spinal cord symptom, a brain/brain stem symptom, or any combination thereof.

The NMOSD-related attack, if characterized by a new or worsening eye symptom may be characterized by eye pain, a new optic nerve lesion, an enlarging optic nerve lesion, blurred vision, loss of vision, or a 5 or more character drop in low-contrast Landolt C Broken Rings Chart. The NMOSD-related attack, if characterized by a new or worsening eye symptom, may further/alternatively meet any one or more of the following criteria: >15-character drop in high-contrast Landolt C Broken Ring Chart from most recent clinical visit as measured in a previously affected eye and no other ophthalmological explanation; reduction of ≥2 steps in counting fingers (CF) to no light perception (NLP) from most recent clinical visit as measured in a previously affected eye and no other ophthalmological explanation; reduction of ≥7 characters in low-contrast Landolt C Broken Ring Chart from most recent clinical visit as measured in either eye alone (monocular) and a new relative afferent pupillary defect (RAPD) in affected eye; reduction of ≥7 characters in low-contrast Landolt C Broken Ring Chart from most recent clinical visit as measured in either eye alone (monocular) and loss of a previously documented RAPD in fellow eye; reduction of ≥5 characters in high-contrast Landolt C Broken Ring Chart from most recent clinical visit as measured in either eye alone (monocular) and a new RAPD in affected eye; reduction of ≥5 characters in high-contrast Landolt C Broken Ring Chart from most recent clinical visit as measured in either eye alone (monocular) and loss of a previously documented RAPD in fellow eye; reduction of ≥1 step in CF to NLP from most recent clinical visit as measured in a previously affected eye and anew RAPD in affected eye; reduction of ≥1 step in CF to NLP from most recent clinical visit as measured in a previously affected eye and loss of a previously documented RAPD in fellow eye; reduction of ≥7 characters in low-contrast Landolt C Broken Ring Chart from most recent clinical visit as measured in either eye alone (monocular) and a new Gadolinium (Gd)-enhancing or new/enlarging T2 MRI lesion in the corresponding optic nerve; reduction of ≥5 characters in high-contrast Landolt C Broken Ring Chart from most recent clinical visit as measured in either eye alone (monocular) and a new Gd-enhancing or new/enlarging T2 MRI lesion in the corresponding optic nerve; reduction of ≥1 step in CF to NLP from most recent clinical visit as measured in a previously affected eye and a new Gd-enhancing or new/enlarging T2 MRI lesion in the corresponding optic nerve.

The NMOSD-related attack, if characterized by a new or worsening spinal cord symptom, may be characterized by deep or radicular pain, extremity paresthesia, weakness, sphincter dysfunction, Lhermitte's sign, a new spinal cord lesion, or an enlarging spinal cord lesion. The NMOSD-related attack, if characterized by a new or worsening spinal cord symptom, may further/alternatively meet any one or more of the following criteria: Worsening of ≥2 points in at least one of the relevant (pyramidal, bladder/bowel, sensory) Functional Systems Scores (FSS) compared with most recent clinical visit; worsening of ≥1 point in EDSS score compared with most recent clinical visit if previous EDSS score ≥5·5; worsening of ≥1 point in at least two of the relevant (pyramidal, bladder/bowel, sensory) FSS compared with most recent clinical visit when the most recent clinical visit score was ≥1 and a new Gd-enhancing or new/enlarging T2 MRI lesion in the spinal cord; worsening of ≥0·5 points in EDSS score compared with most recent visit if previous EDSS score ≥5·5 and a new Gd-enhancing or new/enlarging T2 MRI lesion in the spinal cord.

The NMOSD-related attack, if characterized by a brain or brain stem symptom, may be characterized by nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. The NMOSD-related attack, if characterized by a new or worsening existing symptom, may further/alternatively meet any one or more of the following criteria: isolated (not present at most recent clinical visit) intractable nausea, vomiting, and/or hiccups lasting >48 hours and a new Gd-enhancing or new/enlarging T2 MRI lesion in the brainstem; worsening of ≥2 points in at least one of the relevant (brainstem, cerebellar) FSS compared with most recent clinical visit and a new Gd-enhancing or new/enlarging T2 MRI lesion in the brainstem; or worsening of ≥2 points in at least one of the relevant (cerebral, sensory, pyramidal) FSS (with a score of ≥3 at the current visit) compared with most recent clinical visit and a new Gd-enhancing or new/enlarging T2 MRI lesion in the brain consistent with the clinical presentation.

In methods of reducing NMOSD-related damage in the subject at increased risk therefor, the reduction in NMOSD-related damage may be a reduction in number of MRI lesions or a reduction in rate of increase in new MRI lesions in the at increased risk subject. If the reduction in NMOSD-related damage comprises a reduction in number of MRI lesions, the number of MRI lesions may be reduced by at least 1 lesion, at least 2 lesions, at least 3 lesions, at least 4 lesions, at least 5 lesions, at least 6 lesions, at least 7 lesions, at least 8 lesions, at least 9 lesions, or at least 10 lesions following the administering of a first dose of Inebilizumab or derivative thereof. The reduction in number of MRI lesions may occur within approximately 2 months, approximately 4 months, approximately 6 months, approximately 8 months, approximately 10 months, approximately 12 months, approximately 18 months or approximately 24 months following the administering of a first dose of Inebilizumab or derivative thereof. Alternatively, the reduction in number of MRI lesions may occur within 2 to 12 months, within 4 to 12 months, within 6 to 12 months, within 8 to 12 months, or within 10 to 12 months following the administering of a first dose of Inebilizumab or derivative thereof.

If the reduction in NMOSD-related damage comprises a reduction in rate of increase in number of new MRI lesions, then rate in increase may be reduced by approximately 10%, 10%, approximately 20%, 20%, approximately 30%. 30%, approximately 40%, 40%, approximately 50%, 50%, approximately 60%, 60%, approximately 70%, 70%, approximately 80%, 80%, approximately 90%, 90% or approximately 100% relative to rate of increase in new MRI lesions prior to the administering of a first dose of Inebilizumab or derivative thereof. Alternatively, the reduction in rate of increase in number of MRI lesions may be a reduction in rate of increase of between 25% and 100%, between 50% and 100%, between 75% and 100%, between 25% and 75%, or between 50% and 75% relative to rate of increase in new MRI lesions prior to the administering of a first dose of Inebilizumab or derivative thereof.

The reduction in rate in increase in new MRI lesions may be determined by comparing the rate of increase in new MRI lesions in the subject within a first time period, prior to the administering of Inebilizumab or derivative thereof, to the rate of increase in MRI lesions in the subject in a second time period, following the administering of a first dose of Inebilizumab or derivative thereof to the subject. The first and the second time period may be of an equal length in time and may be of a length in time of approximately 6 months, 6 months, approximately 12 months, 12 months, approximately 18 months, 18 months, approximately 24 months, 24 months, approximately 30 months, 30 months, approximately 36 months, 36 months, approximately 42 months, 42 months, approximately 48 months, 48 months, approximately 54 months, 54 months, approximately 60 months, 60 months, approximately 6 years, 6 years, approximately 7 years, 7 years, approximately 8 years, 8 years, approximately 9 years, 9 years, approximately 10 years, or 10 years. It will be understood that the first and the second time period need not be exactly the same length in time, i.e., need not be exactly the same number of days. Rather, it will be understood that the first and the second time period may be considered to be of equal length if the number of days of the first time period is greater or lesser than 10%, 8%, 6%, 4%, 2%, or 1% the number of days in the second time period.

The first time period may end at most 1 day, at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days or at most 1 month before the administering of Inebilizumab or the derivative thereof. The second time period may begin the day of the administering of a first dose of Inebilizumab or the derivative thereof. Alternatively, the second time period may begin at most 2 days, at most 3 days, at most 4 days, at most 5 days, at most 6 days, at most 7 days, at most 8 days, at most 9 days, at most 10 days, at most 11 days, at most 12 days, at most 13 days, at most 14 days following the administering of a first dose of Inebilizumab or derivative thereof.

The MRI lesions, reduced in number or reduced in rate of increase, in methods of reducing NMOSD-related damage in the subject at increased risk therefor, may be brain lesions, brainstem lesions, spinal cord lesions, optic nerve lesions, or any combination of any two or more of brain, brainstem, spinal cord, and optic nerve lesions. The MRI lesions may be clinically symptomatic lesions or clinically asymptomatic lesions. The MRI lesions may be detected as T2 lesions and/or may be detected using gadolinium as a contrast medium and/or may be detected as gadolinium T1 lesions.

In methods of reducing NMOSD-related damage in the subject at increased risk therefor, the reduction in NMOSD-related damage may comprise an improvement in EDSS score or a reduction in rate of worsening in EDSS score in the at increased risk subject. If the reduction in NMOSD-related damage comprises an improvement in the subject's EDSS score, then the improvement may be a decrease in the subject's EDSS score of at least 0.5 points, or at least 1 point, or at least 1.5 points, or at least 2 points following the administering of Inebilizumab or derivative thereof. The decrease in the subject's EDSS score may occur within 2 weeks, 1 month, 1.5 months, 2 months, 2.5 months, or 3 months following the administering of a first dose of Inebilizumab or derivative thereof. The decrease in the subject's EDSS score of the at least 0.5, at least 1, at least 1.5, or at least 2 points may be a decrease that, once initiated, may continue for a period of time of approximately 1 month, 1 month, approximately 2 months, 2 months, approximately 3 months, 3 months, approximately 4 months, 4 months, approximately 5 months, 5 months, approximately 6 months, 6 months, approximately 9 months, 9 months, approximately 12 months, 12 months, approximately 18 months, 18 months, approximately 24 months, or 24 months. It will be understood that any continued decrease in EDSS score is a reference to the subject's EDSS score being decreased relative to the subject's EDSS score prior to the administering of a first dose of Inebilizumab or derivative thereof, e.g., no requirement that the subject's EDSS score be decreased to be at the same number or to the same degree throughout the entire continued period of time.

If the reduction in NMOSD-related damage comprises a reduction in rate of worsening in EDSS score, then the reduction in rate of worsening in EDSS score in the at increased risk subject may be, if the subject has a baseline EDSS score of 0, a worsening to at most an EDSS score of 0.5, an EDSS score of at most 1, an EDSS score of at most 1.5, or an EDSS score of at most 2 over a period of time of at least 6 months, 9 months, 1 year, 1.5 years. 2 years, 3 years, 4 years, 5 years, 7.5 years, or 10 years. The reduction in rate of worsening in EDSS score in the at increased risk subject may be, if the subject has a baseline EDSS score of 1 to 5, a worsening of the subject's EDSS score by 0.5 points or by no more than 1 point over a period of time. The period of time in which the subject with the baseline score of 1 to 5 worsens by the 0.5 points, or by the no more than 1 point, may be a period of time of at least 6 months, 9 months, 1 year, 1.5 years, 2 years, 3 years, 4 years, 5 years, 7.5 years, or 10 years. The reduction in rate of worsening in EDSS score in the at increased risk subject following administering Inebilizumab or the derivative thereof may be, if the subject has a baseline EDSS score of 5.5 or more, a worsening of the subject's EDSS score by no more than 0.5 points over a period of time. The period of time in which the subject with the baseline score of 5.5 worsens by the no more than 0.5 points may be a period of time of at least 6 months, 9 months, 1 year, 1.5 years, 2 years, 3 years, 4 years, 5 years, 7.5 years, or 10 years. The subject's baseline EDSS score may be determined approximately 1 month, 2 weeks, 1 week, 3 days, 2 days, or 1 day prior to the administering of a first dose of Inebilizumab or derivative thereof.

In methods provided herein, NMOSD relapse is prevented in a subject diagnosed with NMOSD. The present disclosure also provides methods of reducing the likelihood of NMOSD relapse in a subject diagnosed with NMOSD. The subject may be identified as a subject for preventing NMOSD relapse or reducing the likelihood of NMOSD relapse if the subject comprises a sGFAP concentration of less than about 165 pg/mL, about 166 pg/mL, about 167 pg/mL, about 168 pg/mL, about 169 pg/mL, about 170 pg/mL, about 171 pg/mL, about 172 pg/mL, about 173 pg/mL, about 174 pg/mL, about 175 pg/mL, about 176 pg/mL, or about 181 pg/mL.

A subject may be identified as a subject for preventing NMOSD relapse or reducing the likelihood of NMOSD relapse if the subject comprises a sGFAP concentration of about 165 pg/mL, 165 pg/mL, about 166 pg/mL, 166 pg/mL, about 167 pg/mL, 167 pg/mL, about 168 pg/mL, 168 pg/mL, about 169 pg/mL, 169 pg/mL, about 170 pg/mL, 170 pg/mL, about 171 pg/mL, 171 pg/mL, about 172 pg/mL, 172 pg/mL, about 173 pg/mL, 173 pg/mL, about 174 pg/mL, 174 pg/mL, about 175 pg/mL, 175 pg/mL, about 176 pg/mL, 176 pg/mL, about 181 pg/mL, or 181 pg/mL or less. Furthermore, the subject may be identified as a subject for preventing or reducing the likelihood of NMOSD relapse if the subject comprises a sGFAP concentration of between about 165 pg/mL and about 181 pg/mL, between about 167 pg/mL and about 175 pg/mL, between about 168 pg/mL and about 174 pg/mL, or between about 169 pg/mL and about 173 pg/mL or less. Further, the subject may be identified as a subject for preventing or reducing the likelihood of NMOSD relapse if the subject comprises an sGFAP concentration that is less than approximately 2 standard deviations above or 3 standard deviations above a healthy donor's mean sGFAP concentration. In aspects, the subject may be identified as a subject for preventing or reducing the likelihood of NMOSD relapse if the subject comprises a sGFAP concentration that is approximately, or about, 170 pg/mL, e.g., 170 pg/mL, or less. It will be understood that an approximate sGFAP concentration, e.g., of 170 pg/mL, may be an sGFAP concentration that takes into account any deviation or variation, e.g., from 170 pg/mL, caused by a device employed to measure sGFAP concentration. e.g., device calibration, or sample handling or processing leading up to measurement of sGFAP concentration.

A subject identified as a subject for preventing NMOSD relapse, comprising a sGFAP concentration of less than about 165 pg/mL, 165 pg/mL, about 166 pg/mL, 166 pg/mL, about 167 pg/mL, 167 pg/mL, about 168 pg/mL, 168 pg/mL, about 169 pg/mL, 169 pg/mL, about 170 pg/mL, 170 pg/mL, about 171 pg/mL, 171 pg/mL, about 172 pg/mL, 172 pg/mL, about 173 pg/mL, 173 pg/mL, about 174 pg/mL, 174 pg/mL, about 175 pg/mL, 175 pg/mL, about 176 pg/mL, 176 pg/mL, about 181 pg/mL, or 181 pg/mL or less, may further not have experienced an NMOSD-related attack in at least 2 months, at least 3 months, at least 4 months, at least 5 months, or at least 6 months.

In aspects, a subject with NMOSD is identified as a subject for treatment with Inebilizumab if the subject has an increase in serum Neurofilament light chain (Nfl) levels over baseline levels of the subject or as compared to a control subject. In certain aspects, a subject identified as at increased risk for NMOSD-related disability if the subject has an increase in serum NfL levels over baseline levels or as compared to a control subject. In aspects, a composition that comprises Inebilizumab or a derivative thereof is administered to a subject diagnosed with NMOSD with an increase in serum Nfl levels over baseline levels or as compared to a control subject. In aspects, subjects diagnosed with NMOSD with an increase in serum Nfl levels are treated with Inebilizumab or a derivative regardless of if they have had an attack. In aspects, provided are methods of treating NMOSD in a subject, the methods comprising administering a composition that comprises Inebilizumab to a subject with an increase in serum Nfl levels over a baseline level of the subject or as compared to a control subject.

In aspects, the disclosure provides methods of treating NMOSD in a subject, the methods comprising a therapy or administering a therapeutic to a subject with an increase in serum Nfl levels over a baseline level or as compared to a control subject. In aspects, the therapy or therapeutic comprises administering one or more of Eculizumab, Satralizumab, Ublituximab, Ravulizumab, Rituximab, azathioprine, mycophenolate mofetil or low dose corticosteroids. In aspects, a therapeutic is selected from the group consisting of: Eculizumab, Satralizumab, Ublituximab, Ravulizumab, Rituximab, azathioprine, mycophenolate mofetil, and low dose corticosteroid.

In aspects, “baseline” levels of serum Nfl refer to the level of serum Nfl measured at a time before an NMOSD-related attack. In aspects, the subject has an about 1.0, about 1.1, about 1.2, about 1.3, about 1.4, about 1.5, about 1.6, about 1.7, about 1.8, about 1.9, about 2.0 or greater fold change over baseline levels. In aspects, subjects with an increase in serum Nfl levels over baseline levels are treated with a composition that comprises Inebilizumab or a derivative thereof as descried in any method disclosed herein.

An NMOSD-related attack, which may further not have been experienced by the subject, may be an attack characterized by the appearance of a new NMOSD symptom or the worsening of an existing NMOSD symptom. If such an NMOSD-related attack is characterized by a new or worsening existing NMOSD symptom, the symptom may be an eye symptom, a spinal cord symptom, a brain/brain stem symptom, or any combination thereof.

If the subject has further not experienced a new or worsening eye symptom, the new or worsening eye symptom may be eye pain, a new optic nerve lesion, an enlarging optic nerve lesion, blurred vision, loss of vision, or a 5 or more character drop in low-contrast Landolt C Broken Rings Chart. If the subject has further not experienced an NMOSD-related attack, then the subject may not have experienced a new or worsening existing eye symptom that further/alternatively meeting any one or more of the following criteria: >15-character drop in high-contrast Landolt C Broken Ring Chart from most recent clinical visit as measured in a previously affected eye and no other ophthalmological explanation; reduction of ≥2 steps in CF to NLP from most recent clinical visit as measured in a previously affected eye and no other ophthalmological explanation; reduction of ≥7 characters in low-contrast Landolt C Broken Ring Chart from most recent clinical visit as measured in either eye alone (monocular) and a new relative afferent pupillary defect (RAPD) in affected eye; reduction of ≥7 characters in low-contrast Landolt C Broken Ring Chart from most recent clinical visit as measured in either eye alone (monocular) and loss of a previously documented RAPD in fellow eye; reduction of ≥5 characters in high-contrast Landolt C Broken Ring Chart from most recent clinical visit as measured in either eye alone (monocular) and a new RAPD in affected eye; reduction of ≥5 characters in high-contrast Landolt C Broken Ring Chart from most recent clinical visit as measured in either eye alone (monocular) and loss of a previously documented RAPD in fellow eye; reduction of ≥1 step in CF to NLP from most recent clinical visit as measured in a previously affected eye and a new RAPD in affected eye; reduction of ≥1 step in CF to NLP from most recent clinical visit as measured in a previously affected eye and loss of a previously documented RAPD in fellow eye; reduction of ≥7 characters in low-contrast Landolt C Broken Ring Chart from most recent clinical visit as measured in either eye alone (monocular) and a new Gd-enhancing or new/enlarging T2 MRI lesion in the corresponding optic nerve; reduction of ≥5 characters in high-contrast Landolt C Broken Ring Chart from most recent clinical visit as measured in either eye alone (monocular) and a new Gd-enhancing or new/enlarging T2 MRI lesion in the corresponding optic nerve; reduction of ≥1 step in CF to NLP from most recent clinical visit as measured in a previously affected eye and a new Gd-enhancing or new/enlarging T2 MRI lesion in the corresponding optic nerve.

If the subject has further not experienced an NMOSD-related attack, then the subject may not have experienced anew or worsening spinal cord symptom, such as a deep or radicular pain, extremity paresthesia, weakness, sphincter dysfunction, Lhermitte's sign, a new spinal cord lesion, or an enlarging spinal cord lesion. If the subject has not experienced an NMOSD-related attack, the subject may not have experienced a new or worsening existing spinal cord symptom that further/alternatively meets any one or more of the following criteria: Worsening of ≥2 points in at least one of the relevant (pyramidal, bladder/bowel, sensory) FSS compared with most recent clinical visit; worsening of ≥1 point in EDSS score compared with most recent clinical visit if previous EDSS score ≥5·5; worsening of ≥1 point in at least two of the relevant (pyramidal, bladder/bowel, sensory) FSS compared with most recent clinical visit when the most recent clinical visit score was ≥1 and a new Gd-enhancing or new/enlarging T2 MRI lesion in the spinal cord; worsening of ≥0·5 points in EDSS score compared with most recent visit if previous EDSS score ≥5·5 and a new GD-enhancing or new/enlarging T2 MRI lesion in the spinal cord

If the subject has further not experienced an NMOSD-related attack, then the subject may not have experienced a new or worsening brain or brain stem symptom such as nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. If the subject has further not experienced an NMOSD-related attack, the subject may not have experienced a new or worsening existing brain or brain stem symptom that further/alternatively meets any one or more of the following criteria: isolated (not present at most recent clinical visit) intractable nausea, vomiting, and/or hiccups lasting >48 hours and a new Gd-enhancing or new/enlarging T2 MRI lesion in the brainstem; worsening of ≥2 points in at least one of the relevant (brainstem, cerebellar) FSS compared with most recent clinical visit and a new Gd-enhancing or new/enlarging T2 MRI lesion in the brainstem; or worsening of ≥2 points in at least one of the relevant (cerebral, sensory, pyramidal) FSS (with a score of ≥3 at the current visit) compared with most recent clinical visit and a new Gd-enhancing or new/enlarging T2 MRI lesion in the brain consistent with the clinical presentation.

If the subject is identified as a subject for preventing or reducing the likelihood of NMOSD relapse, then the subject may be administered a composition that comprises Inebilizumab or a derivative thereof. The administration of Inebilizumab or the derivative thereof may be intravenously at a dose of 300 mg every six months.

The administering of Inebilizumab or a derivative thereof to the subject results in the preventing NMOSD relapse or reducing the likelihood of NMOSD relapse in the subject. The preventing NMOSD relapse or reducing the likelihood of NMOSD relapse in the subject may be a prevention of NMOSD-related attacks in the subject. The preventing NMOSD relapse or reducing the likelihood of NMOSD relapse in the subject may, alternatively, prevent the worsening of any one or more NMOSD-related symptoms in the subject, even if the one or more NMSOD-related symptoms are not associated with an NMOSD-related attack.

If the preventing NMOSD relapse or reducing the likelihood of NMOSD relapse in the subject prevents the worsening of any one or more NMOSD-related symptoms in the subject, the NMOSD-related symptoms may be clinical or may be subclinical symptoms. The NMOSD-related symptoms may comprise one or more eye symptoms, spinal cord symptoms, brain symptoms or brain step symptoms. If the one or more NMOSD-related symptoms comprise an eye symptom, the eye symptom may be eye pain, a new optic nerve lesion, an enlarging optic nerve lesion, blurred vision, loss of vision, or a 5 or more character drop in low-contrast Landolt C Broken Rings Chart. If the one or more NMOSD-related symptoms comprise a spinal cord symptom, the NMOSD-related symptoms may be deep or radicular pain, extremity paresthesia, weakness, sphincter dysfunction, Lhermitte's sign, a new spinal cord lesion, or an enlarging spinal cord lesion. If the one or more NMOSD-related symptoms comprise a brain or brain stem symptom, the NMOSD-related symptoms may be nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. Further, the NMOSD-related symptoms may include any other symptom or criteria described as characterizing an NMOSD-related attack, the absence of which identified a subject as a subject for preventing relapse.

The preventing NMOSD relapse or reducing the likelihood of NMOSD relapse in the subject may result in a reduction in MRI lesions in the subject. If the preventing or reducing the likelihood of relapse in the subject results in the reduction in MRI lesions in the subject, the reduction in MRI lesions may refer to a reduction in the number of MRI lesions in the subject, a reduction in the number of enlarging MRI lesions in the subject, or a reduction in the combined number of MRI lesions and enlarging MRI lesions in the subject. The reduction in MRI lesions in the subject may be a reduction of at least 1, at least 2, at least 3, at least 4, at least 5, at least 6, at least 7, at least 8, at least 9, or at least 10 lesions. The reduction in MRI lesions in the subject may occur within approximately 2 months, approximately 4 months, approximately 6 months, approximately 8 months, approximately 10 months, approximately 12 months, approximately 18 months or approximately 24 months of the administering of a first dose of Inebilizumab or derivative thereof. Alternatively, the reduction in number of MRI lesions may occur within 2 to 12 months, within 4 to 12 months, within 6 to 12 months, within 8 to 12 months, or within 10 to 12 months of the administering of a first dose of Inebilizumab or derivative thereof. The MRI lesions may be lesions in any one or more of the optic nerve, spinal cord, brain or brain stem of the subject. The MRI lesions may be asymptomatic MRI lesions.

In certain aspects, a composition that comprises Inebilizumab or a derivative is administered to a subject who is suspected of having NMOSD. A subject is suspected of having NMOSD if the subject has one or more NMOSD-related symptoms. The NMOSD-related symptoms may be clinical or may be subclinical symptoms. The NMOSD-related symptoms may comprise one or more eye symptoms, spinal cord symptoms, brain symptoms or brain step symptoms. If the one or more NMOSD-related symptoms comprise an eye symptom, the eye symptom may be eye pain, a new optic nerve lesion, an enlarging optic nerve lesion, blurred vision, loss of vision, or a 5 or more character drop in low-contrast Landolt C Broken Rings Chart. If the one or more NMOSD-related symptoms comprise a spinal cord symptom, the NMOSD-related symptoms may be deep or radicular pain, extremity paresthesia, weakness, sphincter dysfunction, Lhermitte's sign, a new spinal cord lesion, or an enlarging spinal cord lesion. If the one or more NMOSD-related symptoms comprise a brain or brain stem symptom, the NMOSD-related symptoms may be nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. Further, the NMOSD-related symptoms may include any other symptom or criteria described as characterizing an NMOSD-related attack, the absence of which identified a subject as a subject for preventing relapse. In aspects, a composition that comprise Inebilizumab is administered to a subject suspected of having NMOSD with an increase in serum Nfl levels over baseline levels. In aspects, a therapy is administered to a subject suspected of having NMOSD with an increase in serum Nfl levels over baseline levels or as compared to a control subject. In aspects, the therapeutic comprises one or more of Eculizumab, Satralizumab, Ublituximab, Ravulizumab, Rituximab, azathioprine, mycophenolate mofetil or low dose corticosteroids.

The prevention or reduction in likelihood of NMOSD relapse in the subject may result in an improvement in EDSS score in the subject. If the prevention or reduction in likelihood of NMOSD relapse in the subject results in an improvement in the subject's EDSS score, then the improvement may be a decrease in the subject's EDSS score of at least 0.5 points, or at least 1 point, or at least 1.5 points, or at least 2 points following the administering of a first dose of Inebilizumab or derivative thereof. The decrease in the subject's EDSS score may begin within 2 weeks, 1 month, 1.5 months, 2 months, 2.5 months, or 3 months following the administering of a first dose of Inebilizumab or derivative thereof. The decrease in the subject's EDSS score of the at least 0.5, at least 1, at least 1.5, or at least 2 points may be a decrease that, once initiated, may continue for a period of time of approximately 1 month, 1 month, approximately 2 months, 2 months, approximately 3 months, 3 months, approximately 4 months, 4 months, approximately 5 months, 5 months, approximately 6 months, 6 months, approximately 9 months. 9 months, approximately 12 months, 12 months, approximately 18 months. 18 months, approximately 24 months, or 24 months. It will be understood that any continued decrease in EDSS score is a reference to the subject's EDSS score being decreased relative to the subject's EDSS score prior to administering of a first dose of Inebilizumab or derivative thereof, e.g., no requirement that the subject's EDSS score be decreased to be at the same number or to the same degree throughout the entire continued period of time.

The preventing NMOSD relapse or reducing the likelihood of NMOSD relapse in the subject identified as a subject for preventing or reducing the likelihood of NMOSD relapse may be for a time period of at least 1 year from the administering of a first dose of Inebilizumab or derivative thereof. Alternatively, preventing or reducing the likelihood of NMOSD relapse in the subject identified as a subject for preventing or reducing the likelihood of NMOSD relapse may be for a time period of at least 1.5 year, at least 2 years, at least 2.5 years, at least 3 years, at least 3.5 years, at least 4 years, at least 4.5 years, at least 5 years, or at least 10 years from the administering of a first dose of Inebilizumab or derivative thereof.

In the methods of preventing NMOSD relapse or reducing the likelihood NMOSD relapse, as a result of the administering Inebilizumab or derivative thereof, the subject's sGFAP concentration may decrease. If the subject's sGFAP concentration decreases, the decrease may be a decrease of 2% to 30%, 5% to 25%, 5% to 20%, 10% to 20%, 10% to 30%, or 5% to 30%. Alternatively, the subject's sGFAP concentration may decrease by approximately 2%, 2%, approximately 5%. 5%, approximately 10%, 10%, approximately 15%, 15%, approximately 20%, 20%, approximately 25%, 25%, approximately 30%, or 30%. The subject's decrease in sGFAP concentration may be a decrease that, once initiated, may continue for a period of time of approximately 1 month, 1 month, approximately 2 months, 2 months, approximately 3 months, 3 months, approximately 4 months, 4 months, approximately 5 months, 5 months, approximately 6 months, 6 months, approximately 9 months, 9 months, approximately 12 months, 12 months, approximately 18 months, 18 months, approximately 24 months, or 24 months. It will be understood that any continued decrease in sGFAP concentration is a reference to the subject's sGFAP concentration being decreased relative to the subject's sGFAP concentration prior to administering of a first dose of Inebilizumab or derivative thereof, i.e., there is no requirement that the subject's sGFAP concentration be decreased at the same number or to further decrease throughout the entire continued period of time.

Described herein are also methods of suppressing a NMOSD-related attacked in a subject diagnosed with NMSOD. In such methods, the subject is identified as being at risk for an NMOSD-related attack, e.g., is identified as an at-risk subject, if the subject comprises an increase in sGFAP concentration relative to his or her baseline sGFAP concentration or as compared to a control subject. The subject's baseline sGFAP concentration, against which an increase in sGFAP concentration identifies the subject as at-risk, may be the subject's sGFAP concentration at any time he or she is not experiencing an NMOSD-related attack and not within one week, or two weeks, or three weeks of experiencing an NMOSD-related attack.

The increase in sGFAP concentration relative to baseline sGFAP concentration, which may identify a subject as being an at-risk subject, may differ depending on whether the subject is undergoing a treatment for NMOSD that comprises Inebilizumab or a derivative thereof. If the subject is not undergoing a treatment for NMOSD that comprises Inebilizumab or a derivative thereof, the subject may be identified as at-risk for an NMOSD-related attack if the subject's sGFAP concentration increases at least 25-fold, 25-fold, at least 20-fold, 20-fold, at least 15-fold, 15-fold, at least 10-fold, 10-fold, at least 5-fold, 5-fold, at least 2-fold, 2-fold, between 25-fold and 5-fold, between 20-fold and 5-fold, between 15-fold and 5-fold, between 10-fold and 5-fold, between 25-fold and 10-fold, between 25-fold and 15-fold, or between 25-fold and 20-fold relative to baseline or as compared to a control subject. In aspects, the increase in sGFAP concertation occurs or is detected over a period of about: 1 month, 2 months. 3 months, 4 months, 5 months, 6 months, 7 months, 8 months, 9 months, 10 months, 11 months, 12 months, 13 months, 14 months, 15 months, 16 months, 17 months, 18 months, 19 months, 20 months. 21 months, 22 months, 23 months, 24 months, 25 months, 26 months, 27 months, 28 months, 29 months, 30 months, 31 months, 32 months, 33 months, 34 months, 35 months, or 36 months. If the subject's treatment for NMOSD does not comprise Inebilizumab or a derivative thereof, then the subject may not be undergoing any treatment for NMOSD, or the subject may be undergoing treatment for NMOSD with any non-Inebilizumab or non-Inebilizumab derivative treatment known in the art to have been used for the treatment of NMOSD, e.g., azathioprine, mycophenolate mofetil or low dose corticosteroids.

If the subject is undergoing a treatment for NMOSD that comprises administration of Inebilizumab or a derivative thereof, the subject may be identified as at-risk for an NMOSD-related attack if the subject's sGFAP concentration, relative to baseline, increases by at least 25%, 25%, at least 50%, 50%, at least 75%, 75%, at least 100%, 100%, at least 125%, 125%, at least 150%, 150%, at least 175%, 175%, at least 200%, 200%, between 25% and 200%, between 50% and 200%, between 75% and 200%, between 100% and 200%, between 125% and 200%, between 150% and 200%, between 175% and 200%, between 25% and 50%, between 25% and 75%, between 25% and 100%, between 25% and 125%, between 25% and 150%, between 25% and 175%, between 50% and 150%, between 75% and 125%, between 100% and 200%, or between 50% and 100%.

If the subject is identified as at-risk for an NMOSD-related attack, i.e., as having an increase in sGFAP concentration relative to baseline sGFAP concentration, a therapeutic may be administered to the at-risk subject. Administration of the therapeutic to the at-risk subject may occur at most one week following the subject's identification as an at-risk subject. Alternatively, administration of the therapeutic to the at-risk subject may occur at most 6 days, at most 5 days, at most 4 days, at most 3 days, at most 2 days or at most 1 day following the subject's identification as an at-risk subject.

In aspects, a therapeutic is administered to an at-risk subject at most about one week, at most 6 days, at most 5 days, at most 4 days, at most 3 days, at most 2 days or at most 1 day following the subject's identification as at-risk. In aspects, a therapeutic may comprise one or more of a high dose steroid, plasmapheresis, immunoadsorption, a complement inhibitor, or any other agent not included as part of the at-risk subject's treatment regimen at the time the subject is identified as being at-risk. In aspects, a therapeutic is selected from the group consisting of: a high dose steroid, plasmapheresis, immunoadsorption, and a complement inhibitor.

In aspects, a therapy is administered to an at-risk subject within at most one week, at most 6 days, at most 5 days, at most 4 days, at most 3 days, at most 2 days or at most 1 day following the subject's identification as at-risk. In aspects, a therapeutic may comprise or may further comprise a composition comprising Inebilizumab or a derivative thereof, if the at-risk subject's treatment regimen at the time of identification, did not comprise a composition comprising Inebilizumab or a derivative thereof. If the therapy administered to the at-risk subject comprises or further comprises a composition comprising Inebilizumab or a derivative thereof, Inebilizumab or the derivative thereof may be administered to the at-risk subject at a dose of approximately 300 mg. An approximately 300 mg dose may be a dose of about 250 mg to about 350 mg, it may be a dose of about 275 mg to about 325 mg, it may be a dose of about 290 mg to about 310 mg, it may be a dose of about 205 mg to about 305 mg, or it may be a dose of 275 mg, 280 mg, 285 mg, 290 mg, 295 mg, 300 mg, 305 mg, 310 mg, 315 mg, 320 mg, or 325 mg.

If the therapy administered to the at-risk subject is Inebilizumab or a derivative thereof, then the subject may continue therapy with Inebilizumab or the derivative thereof following the administering of a first dose of Inebilizumab or the derivative thereof. If the subject continues therapy with Inebilizumab or the derivative thereof, a second Inebilizumab or derivative thereof dose may be administered to the at-risk subject approximately two weeks after the first dose, and third and subsequent doses may be administered to the at-risk subject at a time interval of approximately 6 months following their preceding dose. By the approximately 6 months, it should be understood that the third and subsequent Inebilizumab or derivative thereof doses may be administered to the subject identified as at-risk at a time interval of approximately 180 days, between 170 and 190 days, between 175 and 185 days, between 175 and 190 days, between 170 and 185 days, approximately 26 weeks, approximately 25 weeks, approximately 27 weeks, between 25 and 27 weeks, between 25 and 26 weeks, or between 26 and 27 weeks following their preceding dose.

The suppressing the NMOSD-related attack in the at-risk subject may reduce the likelihood of or prevent the NMOSD-related attack in the at-risk subject. If the suppressing the NMOSD-related attack reduces the likelihood of the NMOSD-related in the at-risk subject, then the reduced likelihood may decrease the risk of the at-risk subject suffering an NMOSD-related attack by approximately 10%, 10%, approximately 20%, 20%, approximately 30%, 30%, approximately 40%, 40%, approximately 50%, 50%, approximately 60%, 60%, approximately 70% 70%, approximately 80%, 80%, approximately 90%, or 90%. The suppressing the NMOSD-related attack in the at-risk subject may, alternatively, prevent the subject from experiencing an NMOSD-related attack or symptoms of an NMOSD-related attack.

The suppressing the NMOSD-related attack in the at-risk subject may result in a reduction in likelihood or prevention of the at-risk subject suffering any NMOSD-related attack graded major in severity. An NMOSD-related attack graded major in severity may be an NMOSD-related attack that resultantly requires intensive therapeutic intervention, interrupts usual activities of daily living, significantly affects the subject's clinical status, or requires in-subject hospitalization. An NMOSD-related attack graded as major in severity may be an NMOSD-related attack that, if it affects brain, results in an increase in the subject's brain domain subscale score of 2 or more points when compared to the subject's brain domain subscale score prior to the NMOSD-related attack. An NMOSD-related attack graded as major in severity may be an NMOSD-related attack that, if it affects any of the subject's optic nerve, spinal cord or brainstem, results in the affected domain's subscale score increasing by ≥3 points when compared the affected domain's subscale score prior the attack, wherein the affected domain's subscale score had been less than 2 prior to the NMOSD-related attack. An NMOSD-related attack graded as major in severity may be an NMOSD-related attack that, if it affects any of the subject's optic nerve, spinal cord or brainstem, results in the affected domain's subscale score increasing by ≥2 when compared the affected domain's subscale score prior the attack, wherein the affected domain's subscale score had been ≥2 prior to the NMOSD-related attack. Domain subscale scores may be determined according to the domain numerical assignments presented in Table 2.

The suppressing the NMOSD-related attack in the at-risk subject may result in a recovery from the NMOSD-related attack that is graded as major. A recovery from an NMOSD-related attack that affects brain may be graded as major if the recovery from the attack comprises an improvement in the subject's brain subscale score of greater than 1 in a follow-up to the attack. A recovery from an NMOSD-related attack that affects a subjects optic nerve, spinal cord or brainstem may be graded as major if the recovery from the attack improves the subject's affected (optic nerve, spinal cord or brainstem) domain subscale score by 22 in a follow-up to the attack. The follow up to the attack, at which time a subject's subscale score in the affected domain is assessed for determining the grading of the recovery, may take place approximately 2 weeks, 4 weeks, 6 weeks, 8 weeks, 10 weeks. 12 weeks, 14 weeks, or 16 weeks after the NMOSD-related attack. Domain subscale scores may be determined according to the domain numerical assignments presented in Table 2.

In the methods of suppressing a NMOSD-related attack, the NMOSD-related attacks may be attacks characterized by the appearance of a new NMOSD symptom or the worsening of an existing NMOSD symptom. The new or worsening existing symptom which characterizes the NMOSD-related attack may be an eye symptom, a spinal cord symptom, a brain/brain stem symptom, or any combination thereof.

If the NMOSD-related attack is characterized by an eye symptom, the eye symptom may be eye pain, a new optic nerve lesion, an enlarging optic nerve lesion, blurred vision, loss of vision, or a 5 or more character drop in low-contrast Landolt C Broken Rings Chart. If the NMOSD-related attack is characterized by a new or worsening existing eye symptom, it may further/alternatively meet any one or more of the following criteria: >15-character drop in high-contrast Landolt C Broken Ring Chart from most recent clinical visit as measured in a previously affected eye and no other ophthalmological explanation; reduction of ≥2 steps in CF to NLP from most recent clinical visit as measured in a previously affected eye and no other ophthalmological explanation; reduction of ≥7 characters in low-contrast Landolt C Broken Ring Chart from most recent clinical visit as measured in either eye alone (monocular) and a new RAPD in affected eye; reduction of ≥7 characters in low-contrast Landolt C Broken Ring Chart from most recent clinical visit as measured in either eye alone (monocular) and loss of a previously documented RAPD in fellow eye; reduction of ≥5 characters in high-contrast Landolt C Broken Ring Chart from most recent clinical visit as measured in either eye alone (monocular) and a new RAPD in affected eye; reduction of ≥5 characters in high-contrast Landolt C Broken Ring Chart from most recent clinical visit as measured in either eye alone (monocular) and loss of a previously documented RAPD in fellow eye; reduction of ≥1 step in CF to NLP from most recent clinical visit as measured in a previously affected eye and a new RAPD in affected eye; reduction of ≥1 step in CF to NLP from most recent clinical visit as measured in a previously affected eye and loss of a previously documented RAPD in fellow eye; reduction of ≥7 characters in low-contrast Landolt C Broken Ring Chart from most recent clinical visit as measured in either eye alone (monocular) and a new Gd-enhancing or new/enlarging T2 MRI lesion in the corresponding optic nerve; reduction of ≥5 characters in high-contrast Landolt C Broken Ring Chart from most recent clinical visit as measured in either eye alone (monocular) and a new Gd-enhancing or new/enlarging T2 MRI lesion in the corresponding optic nerve; reduction of ≥1 step in CF to NLP from most recent clinical visit as measured in a previously affected eye and a new Gd-enhancing or new/enlarging T2 MRI lesion in the corresponding optic nerve.

If the NMOSD-related attack is characterized by a spinal cord symptom, the spinal cord symptom may be a deep or radicular pain, extremity paresthesia, weakness, sphincter dysfunction, Lhermitte's sign, a new spinal cord lesion, or an enlarging spinal cord lesion. If the NMOSD-related attack is characterized by a new or worsening spinal cord symptom, it may further/alternatively meet any one or more of the following criteria: Worsening of ≥2 points in at least one of the relevant (pyramidal, bladder/bowel, sensory) FSS compared with most recent clinical visit; worsening of ≥1 point in EDSS score compared with most recent clinical visit if previous EDSS score ≥5·5; worsening of ≥1 point in at least two of the relevant (pyramidal, bladder/bowel, sensory) FSS compared with most recent clinical visit when the most recent clinical visit score was ≥1 and a new Gd-enhancing or new/enlarging T2 MRI lesion in the spinal cord; worsening of ≥0·5 points in EDSS score compared with most recent visit if previous EDSS score ≥5·5 and a new GD-enhancing or new/enlarging T2 MRI lesion in the spinal cord.

If the NMOSD-related attack is characterized by a brain or brain stem symptom, the brain or brain stem symptom may be nausea, double vision, oculomotor palsy, vertigo, intractable vomiting, intractable hiccups, dysarthria, dysphagia, weakness, encephalopathy, hypothalamic dysfunction, a new brain or brain stem lesion, or an enlarging brain or brain stem lesion. If the NMOSD-related attack is characterized by a new or worsening brain/brain stem symptom, it may further/alternatively meet any one or more of the following criteria: isolated (not present at most recent clinical visit) intractable nausea, vomiting, and/or hiccups lasting >48 hours and a new Gd-enhancing or new/enlarging T2 MRI lesion in the brainstem; worsening of ≥2 points in at least one of the relevant (brainstem, cerebellar) FSS compared with most recent clinical visit and a new Gd-enhancing or new/enlarging T2 MRI lesion in the brainstem; or worsening of ≥2 points in at least one of the relevant (cerebral, sensory, pyramidal) FSS (with a score of ≥3 at the current visit) compared with most recent clinical visit and a new Gd-enhancing or new/enlarging T2 MRI lesion in the brain consistent with the clinical presentation.

In methods of suppressing a NMOSD-related attack, the administration of the therapeutic to the subject identified as at-risk may result in a prevention of new MRI lesions in the at-risk subject. If the administration of the therapeutic results in a prevention of new MRI lesions in the at-risk subject, the MRI lesions may be clinically symptomatic or clinically asymptomatic lesions. The MRI lesions may be brain lesions, brainstem lesions, spinal cord lesions, optic nerve lesions, or any combination of any two or more of brain, brainstem, spinal cord, and optic nerve lesions. The MRI lesions may be lesions detected as T2 lesions and/or using gadolinium as a contrast medium.

In methods of suppressing a NMOSD-related attack, the administration of the therapeutic to the subject identified as at-risk may result in a reduction in NMOSD-related disability in the at-risk subject. A reduction in NMOSD-related disability may be a reduction in worsening of NMOSD-related disability, or it may be a decrease in NMOSD-related disability, in the at-risk subject. The NMOSD-related disability reduced in the at-risk subject may be a neurological disability or a manifestation of a neurological disability. The NMOSD-related disability reduced in the at-risk subject may be one characterized by one or more of eye pain, a loss of color vision, an overall loss of vision, blurred vision, double vision, overall weakness or paralysis, weakness or paralysis in the arms or legs, radicular pain, uncontrollable hiccups, uncontrollable nausea or vomiting, loss of bladder or bowel control, paralysis, and/or fatigue.

A reduction in worsening in disability in the at-risk subject may be a reduction in worsening of the at-risk subject's EDSS score. A reduction in worsening of the at-risk subject's EDSS score, if the at-risk subject has a baseline EDSS score of 0, may be a worsening of the at-risk subject's EDSS score to a score of 0.5, or to a score of no more than 1, or to a score of no more than 1.5, or to a score of no more than 2 over a period of time. The period of time in which the at-risk subject with the baseline score of 0 worsens to a score of 0.5, to no more than 1, to no more than 1.5, or to no more than 2 may be at least 6 months, 9 months, 1 year, 1.5 years, 2 years, 3 years, 4 years, 5 years, 7.5 years, or 10 years. A reduction in the worsening of the at-risk subject's EDSS score, if the at-risk subject has a baseline EDSS score of 1 to 5, may be a reduction in worsening of the at-risk subject's EDSS score by 0.5 points or by no more than 1 point over a period of time. The period of time over which the at-risk subject with the baseline score of 1 to 5 worsens by 0.5 points, or by no more than 1 point, may be at least 6 months, 9 months, 1 year, 1.5 years, 2 years. 3 years, 4 years, 5 years, 7.5 years, or 10 years. If the reducing the NMOSD-related disability is a reduction in the worsening in the at-risk subject's EDSS score, and the at-risk subject has a baseline EDSS score of 5.5 or more, then the reduction in worsening may be a worsening of the at-risk subject's EDSS score by no more than 0.5 points. The period of time in which the at-risk subject with the baseline score of 5.5 worsens by the no more than 0.5 points may be at least 6 months, 9 months, 1 year, 1.5 years, 2 years, 3 years, 4 years, 5 years, 7.5 years, or 10 years. The at-risk subject's baseline EDSS score may be determined approximately 1 month, 2 weeks. 1 week, 3 days. 2 days, or 1 day prior to the administering of a first dose of Inebilizumab or derivative thereof.

The description further provides for methods of treating NMOSD in a subject. In the methods, a therapeutically effective amount of B cell depleting therapy may be administered to the subject when the subject has a sGFAP concentration of about 160 pg/mL, 160 pg/mL, about 165 pg/mL, 165 pg/mL, about 166 pg/mL, 166 pg/mL, about 167 pg/mL, 167 pg/mL, about 168 pg/mL, about 169 pg/mL, 169 pg/mL, about 170 pg/mL, 170 pg/mL, about 171 pg/mL. 171 pg/mL, about 172 pg/mL, 172 pg/mL, about 173 pg/mL, 173 pg/mL or greater. Furthermore, the therapeutically effective amount of the B cell depleting therapy may be administered to the subject when the subject has a sGFAP concentration of between about 160 pg/mL and about 176 pg/mL, between about 167 pg/mL and about 175 pg/mL, between about 168 pg/mL and about 174 pg/mL, or between about 169 pg/mL and about 173 pg/mL or greater. Further, the therapeutically effect amount of the B cell depleting therapy may be administered to the subject when the subject has a sGFAP concentration that is approximately 2 standard deviations above or 3 standard deviations above a healthy donor's mean sGFAP concentration or greater. In aspects, the subject may have a sGFAP concentration of approximately, or about, 170 pg/mL, e.g., a sGFAP concentration of 170 pg/mL, or greater. It will be understood that a measurement of sGFAP concentration, e.g., of 170 pg/mL, may be an sGFAP concentration that takes into account any deviation or variation, e.g., from 170 pg/mL, caused by a device employed to measure sGFAP concentration, e.g., device calibration, or sample handling or processing leading up to measurement of sGFAP concentration.

The B cell depleting therapy administered to the subject, when the subject has a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater, may be a B cell depleting therapy. A B cell depleting therapy may be anti-CD19 antibody such as VIB551 or a derivative thereof. A B cell depleting therapy may be any therapy that depletes all or a select subset of B cells in the subject. A B cell depleting therapy may be an anti-CD20 antibody such as Rituximab, Ocrelizumab or Ofatumumab. A B cell depleting therapy may be an anti-CD22 antibody such as Epratuzumab. A B cell depleting therapy may inhibit B Lymphocyte Stimulator (BLyS), such as Belimumab, BR3-Fc, AMG-623, or Atacicept.

In methods of treating NMOSD in the subject, when the subject has a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater, the administration of the therapeutically effective amount of the B cell depleting therapy may thereby reduce NMOSD-related damage in the subject, reduce number of NMOSD-related attacks in the subject, reduce likelihood of NMOSD-related attacks in the subject, reduce likelihood of NMOSD-related attacks that are graded as major in severity in the subject, reduce the number of MRI lesions in the subject, reduce the rate of increase in new MRI lesions in the subject, reduce the worsening of EDSS score of the subject, improve the subject's EDSS score, or improve the subject's recovery from an NMOSD-related attack.

Those skilled in the art will recognize, or be able to ascertain using no more than routine experimentation, many equivalents to the aspects described herein. Such equivalents are intended to be encompassed by the appended claims.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

### NUMBERED EMBODIMENTS

Notwithstanding the appended claims, the following numbered embodiments are also contemplated by the instant disclosure.

1. A method of reducing neuromyelitis optica spectrum disorder (NMOSD)-related damage in a patient at increased risk therefor, the method comprising: identifying a patient as at increased risk for NMOSD-related damage, wherein the patient is identified as at increased risk if the patient comprises a serum glial fibrillary astrocytic protein (sGFAP) concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater; administering VIB551 to the at increased risk patient; and reducing the NMOSD-related damage in the at increased risk patient.

2. The method of embodiment 1, wherein the patient is identified as at increased risk if the patient comprises a sGFAP concentration of about 170 pg/mL or greater.

3. The method of embodiment 2, wherein the VIB551 is administered intravenously at a dose of 300 mg every 6 months.

4. The method of any of embodiments 1-3, wherein the reducing NMOSD-related damage comprises reducing number of NMOSD-related attacks in the at increased risk patient relative to a baseline number of NMOSD-related attacks in the at increased risk patient, wherein the baseline number of attacks are determined over a first time period preceding the administering VIB551, wherein the number of attacks reduced by the administering VIB551 are determined over a second time period following the administering VIB551, and wherein the first and the second time period are of equal length.

5. The method of embodiment 4, wherein the first and the second time period are at least one year.

6. The method of any of embodiments 1-3, wherein the reducing the NMOSD-related damage comprises reducing likelihood of NMOSD-related attacks in the at increased risk patient graded major in severity.

7. The method of any of embodiments 1-3, wherein the reducing the NMOSD-related damage comprises preventing NMOSD-related attacks in the at increased risk patient graded major in severity.

8. The method of any of embodiments 1-3, wherein the reducing the NMOSD-related damage comprises reducing number of magnetic resonance imaging (MRI) lesions or reducing rate of increase in new MRI lesions in the at increased risk patient.

9. The method of any of embodiments 1-3, wherein the reducing the NMOSD-related damage comprises reducing rate of worsening of expanded disability status scale (EDSS) score, or improving EDSS score, of the at increased risk patient.

10. A method of preventing NMOSD relapse in a patient diagnosed with NMOSD, the method comprising: identifying the patient as a candidate for preventing NMOSD relapse, wherein the patient is identified as a candidate if the patient: comprises a sGFAP concentration of less than about 165 pg/mL, about 166 pg/mL, about 167 pg/mL, about 168 pg/mL, about 169 pg/mL, about 170 pg/mL, about 171 pg/mL, about 172 pg/mL, about 173 pg/mL, about 174 pg/mL, about 175 pg/mL, about 176 pg/mL, or about 181 pg/mL; administering VIB551 to the candidate; and preventing relapse in the candidate.

11. The method of embodiment 10, wherein the patient is identified as a candidate if the patient comprises a sGFAP concentration of less than about 170 pg/mL.

12. The method of embodiment 11, wherein the VIB551 is administered intravenously at a dose of 300 mg every 6 months.

13. The method of any of embodiments 10-12, wherein the preventing comprises a time period of at least 1 year.

14. The method of any of embodiments 10-12, wherein the administering decreases sGFAP concentration in the candidate relative to baseline sGFAP concentration, and wherein the preventing comprises a time period of at least 2 years.

15. The method of any of embodiments 10-12, wherein the preventing results in a reduction in MRI lesions in the candidate.

16. The method of any of embodiments 10-12, wherein the preventing results in an improvement in EDSS score in the candidate.

17. A method of suppressing a NMOSD-related attack in a patient diagnosed with NMOSD, the method comprising: identifying the patient as at-risk for an NMOSD-related attack, wherein the patient is identified as an at-risk patient if the patient comprises an increase in sGFAP concentration relative to baseline sGFAP concentration; administering a therapy to the at-risk patient, wherein the administering is performed at most one week following the identifying; and suppressing the NMOSD-related attack in the at-risk patient.

18. The method of embodiment 17, wherein the increase in sGFAP concentration comprises an at least 10-fold increase; and wherein the at-risk patient is not undergoing a treatment for NMOSD that comprises VIB551.

19. The method of embodiment 17, wherein the increase in sGFAP concentration comprises an at least 20-fold increase; and wherein the at-risk patient is not undergoing a treatment for NMOSD that comprises VIB551.

20. The method of embodiment 17, wherein the increase in sGFAP concentration comprises an increase of 50% to 150%; and wherein the at-risk patient is undergoing treatment for NMOSD, wherein the treatment comprises VIB551.

21. The method of any of embodiment 17-20, wherein the therapy comprises one or more of steroids, plasmapheresis, immunoadsorption or a complement inhibitor.

22. The method of embodiment 18 or 19, wherein the treatment comprises one or more of Eculizumab, Satralizumab, Ublituximab, Ravulizumab, Rituximab, azathioprine, mycophenolate mofetil or low dose corticosteroids.

23. The method of any one of embodiments 17-20, wherein the administering is performed at most 24 hours following the identifying.

24. The method of any of embodiments 17-20, wherein the suppressing the NMOSD-related attack comprises reducing likelihood of or preventing the NMOSD-related attack.

25. The method of any of embodiments 17-20, wherein the suppressing the NMOSD-related attack comprises reducing likelihood of or preventing the NMOSD-related attack from being graded as major in severity.

26. The method of any of embodiments 17-20, wherein the suppressing the NMOSD-related attack comprises a recovery from the NMOSD-related attack that is graded as a major recovery.

27. The method of any of embodiments 17-20, wherein the suppressing the NMOSD-related attack results in a prevention of new MRI lesions in the at-risk patient.

28. The method of any of embodiments 17-20, wherein the suppressing the NMOSD-related attack results in a reduction in NMOSD-related disability in the at-risk patient.

29. The method of embodiment 28, wherein the reduction in NMOSD-related disability is a reduction in worsening of the at-risk patient's EDSS score.

30. The method of any of embodiments 17-19, wherein the therapy comprises VIB551.

31. A method of treating NMOSD in a subject, the method comprising administering a therapeutically effective amount of a B cell depleting therapy to the subject when the subject has a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater.

32. The method of embodiment 31, wherein the subject has a sGFAP concentration of about 170 pg/mL to 171 pg/mL.

33. The method of embodiment 32, wherein the B cell depleting therapy is VIB551 and the therapeutically effective amount comprises a dose of 300 mg.

34. A method of reducing NMOSD-related disability in a patient diagnosed with NMOSD, the method comprising: identifying a patient as at increased risk for NMOSD-related disability, wherein the patient is identified as at increased risk if the patient has an increase in serum Neurofilament light chain (NfL) levels over a baseline level; and administering VIB551 to the patient.

35. A method of reducing NMOSD-related disability in a patient diagnosed with NMOSD, the method comprising administering VIB551 to a patient with an increase in serum Nfl levels over a baseline level.

36. A method of treating NMOSD in a patient, the method comprising: identifying a patient as at increased risk for NMOSD-related disability, wherein the patient is identified as at increased risk if the patient has an increase in serum Neurofilament light chain (NfL) levels over a baseline level; and administering VIB551 to the patient.

37. A method of treating NMOSD in a patient, the method comprising administering VIB551 to a patient with an increase in serum Nfl levels over a baseline level.

38. The method of any one of embodiments 34-37, wherein the patient has an about 1.0, about 1.1, about 1.2, about 1.3, about 1.4, about 1.5, about 1.6, about 1.7, about 1.8, about 1.9, about 2.0 or greater fold change in serum Nfl over baseline levels.

39. The method of any one of embodiments 34-38, wherein the patient has a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater.

40. A method of treating a patient suspected of having NMOSD, the method comprising administering VIB551 to a patient with an increase in serum Nfl levels over a baseline level and one or more NMOSD-related symptoms.

41. A method of treating a patient suspected of having NMOSD, the method comprising: identifying a patient as having one or more NMOSD-related symptoms; identifying a patient as at increased risk for NMOSD-related disability, wherein the patient is identified as at increased risk if the patient has an increase in serum Neurofilament light chain (NfL) levels over a baseline level; and administering VIB551 to the patient.

42. A method of treating NMOSD in a patient, the method comprising a therapy to a patient with an increase in serum Nfl levels over a baseline level.

43. A method of treating a patient suspected of having NMOSD, the method comprising administering a therapy to a patient with an increase in serum Nfl levels over a baseline level and one or more NMOSD-related symptoms.

44. The method of embodiment 42 or embodiment 43, wherein the therapy comprises one or more of Eculizumab, Satralizumab, Ublituximab, Ravulizumab, Rituximab, azathioprine, mycophenolate mofetil or low dose corticosteroids.

45. A method of reducing neuromyelitis optica spectrum disorder (NMOSD)-related damage in a subject in need thereof, the method comprising administering a composition that comprises Inebilizumab or a derivative thereof to the subject in need thereof, thereby reducing the NMODS-related damage, wherein the subject in need thereof comprises a serum glial fibrillary astrocytic protein (sGFAP) concentration of at least about 160 pg/mL.

46. The method of embodiment 45, wherein the sGFAP concentration is at least about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater.

47. The method of embodiment 46, wherein the sGFAP concentration is at least about 170 pg/mL.

48. The method of any one of embodiments 45-47, wherein the composition that comprises Inebilizumab or the derivative thereof is administered intravenously.

49. The method of embodiment 48, wherein the intravenous administration is at a dose of about 300 mg.

50. The method of any one of embodiments 45-49, wherein the administering is repeated at least twice.

51. The method of embodiment 50, wherein the administering is repeated every 6 months.

52. The method of any of embodiments 45-51, wherein the reducing NMOSD-related damage is determined by at least one of: (a) a reduction in a number of NMOSD-related attacks in the subject in need thereof after the administering as compared to a baseline number of NMOSD-related attacks in the subject in need thereof before the administering; or (b) a reduction in a number of NMOSD-related attacks in the subject in need thereof after the administering as compared to an otherwise comparable control subject lacking the administering.

53. The method of embodiment 52, wherein the baseline number of NMODS-related attacks are determined over a first time period preceding the administering, wherein the number of NMODS-related attacks reduced by the administering are determined over a second time period following the administering, and wherein the first time period and the second time period are of equal length.

54. The method of embodiment 53, wherein the first time period and the second time period are at least one year.

55. The method of any of embodiments 45-54, wherein the reducing the NMOSD-related damage comprises reducing NMOSD-related attacks that are graded major in severity in the subject in need thereof.

56. The method of any of embodiments 45-54, wherein the reducing the NMOSD-related damage comprises eliminating NMOSD-related attacks that are graded major in severity in the subject in need thereof.

57. The method of any of embodiments 45-54, wherein the reducing the NMOSD-related damage in the subject in need thereof comprises: (a) reducing a number of magnetic resonance imaging (MRI) lesions, (b) reducing rate of increase in new MRI lesions, or (c) both (a) and (b).

58. The method of any of embodiments 45-54, wherein the reducing the NMOSD-related damage in the subject in need thereof comprises: (a) reducing a rate of worsening of expanded disability status scale (EDSS) score; or (b) improving the EDSS score.

59. The method of any one of embodiments 45-57, further comprising identifying the subject in need thereof by determining the sGFAP concentration of at least about 160 pg/mL.

60. A method of preventing neuromyelitis optica spectrum disorder (NMOSD) relapse in a subject in need thereof, the method comprising administering a composition that comprises Inebilizumab or a derivative thereof to the subject in need thereof thereby preventing NMODS relapse, wherein the subject in need thereof comprises a serum glial fibrillary acidic protein (sGFAP) concentration of about 165 pg/mL.

61. The method of embodiment 60, wherein the sGFAP concentration is about: 166 pg/mL, 167 pg/mL, 168 pg/mL, 169 pg/mL, 170 pg/mL, 171 pg/mL, 172 pg/mL, 173 pg/mL, or greater.

62. The method of embodiment 61, wherein the sGFAP concentration is about 170 pg/mL.

63. The method of any one of embodiments 59-61, wherein the composition that comprises Inebilizumab or the derivative thereof is administered intravenously.

64. The method of embodiment 63, wherein the intravenous administration is at a dose of about 300 mg.

65. The method of any one of embodiments 59-64, wherein the administering is repeated at least twice.

66. The method of embodiment 65, wherein the administering is repeated every 6 months.

67. The method of any of embodiments 59-66, wherein the preventing lasts for at least 1 year after the administering.

68. The method of any of embodiments 59-66, wherein the preventing lasts for at least 2 years after the administering.

69. The method of any of embodiments 59-68, wherein the administering decreases sGFAP concentration: (a) in the subject in need thereof as compared to sGFAP concentration prior to the administering; (b) in the subject in need thereof as compared to the subject in need thereof's baseline sGFAP concentration; or (c) in an otherwise comparable subject in need thereof lacking the administering.

70. The method of any of embodiments 59-69, wherein the preventing results in a reduction in MRI lesions in the subject in need thereof as determined by: (a) a reduction in a number of the MRI lesions; (b) a reduction in size of the MRI lesions; or (c) both (a) and (b).

71. The method of any of embodiments 59-70, wherein the preventing results in an improvement in EDSS score in the subject in need thereof.

72. A method of suppressing a neuromyelitis optica spectrum disorder (NMOSD)-related attack in a subject diagnosed with NMOSD, the method comprising: (a) identifying the subject as at-risk for an NMOSD-related attack, wherein the subject is identified as an at-risk subject if the subject comprises an increase in sGFAP concentration relative to a baseline sGFAP concentration; and (b) administering a therapeutic to the at-risk subject in an amount effective to suppress the NMODS-related attack, wherein the administering is performed at most one week following the identifying.

73. The method of embodiment 72, wherein the increase in the sGFAP concentration comprises an at least 10-fold increase relative to the baseline sGFAP concentration.

74. The method of embodiment 72, wherein the increase in the sGFAP concentration comprises an at least 20-fold increase relative to the baseline sGFAP concentration.

75. The method of any one of embodiments 72-74, wherein the subject at risk for the NMOSD-related attack is not undergoing a treatment for NMOSD that comprises Inebilizumab or a derivative thereof.

76. The method of embodiment 72, wherein the increase in sGFAP concentration comprises an increase of 50% to 150% relative to the baseline sGFAP concentration; wherein the subject at risk for the NMOSD-related attack is undergoing treatment for NMOSD, and wherein the treatment comprises Inebilizumab or a derivative thereof.

77. The method of any of embodiments 72-76, wherein the therapeutic comprises one or more of a steroid, plasmapheresis, immunoadsorption, or a complement inhibitor.

78. The method of any one of embodiments 72-76, wherein the therapeutic comprises one or more of Eculizumab, Satralizumab, Ublituximab, Ravulizumab, Rituximab, Azathioprine, Mycophenolate Mofetil, or a low dose corticosteroid.

79. The method of any one of embodiments 64-78, wherein the administering is performed at most 24 hours following the identifying.

80. The method of any of embodiments 64-79, wherein the suppressing the NMOSD-related attack comprises: (a) reducing a number of NMODS-related attacks; or (b) preventing a NMOSD-related attack.

81. The method of embodiment 80, comprising (a), wherein the reducing comprises reducing a number of NMODS-related attacks graded as major in severity.

82. The method of any of embodiments 72-79, wherein the suppressing the NMOSD-related attack comprises a recovery from the NMOSD-related attack that is graded as a major recovery.

83. The method of any of embodiments 72-79, wherein the suppressing the NMOSD-related attack results in a prevention of new MRI lesions in the subject at-risk for an NMOSD-related attack.

84. The method of any of embodiments 72-79, wherein the suppressing the NMOSD-related attack results in a reduction in NMOSD-related disability in the subject at-risk for an NMOSD-related attack.

85. The method of embodiment 84, wherein the reduction in NMOSD-related disability is a reduction in worsening of the subject at-risk for an NMOSD-related attack's EDSS score.

86. The method of any of embodiments 72-76, wherein the therapeutic comprises Inebilizumab or a derivative thereof.

87. A method of treating neuromyelitis optica spectrum disorder (NMOSD) in a subject in need thereof, the method comprising administering a therapeutically effective amount of a B cell depleting therapy to the subject in need thereof, wherein the subject has a serum glial fibrillary acidic protein (sGFAP) concentration of about 160 pg/mL.

88. The method of embodiment 87, wherein the sGFAP concentration is about: 165 pg/mL, 166 pg/mL, 167 pg/mL, 168 pg/mL, 169 pg/mL, 170 pg/mL, 171 pg/mL, 172 pg/mL, 173 pg/mL, or greater.

89. The method of embodiment 88, wherein the subject in need thereof has a sGFAP concentration of about 170 pg/mL to 171 pg/mL.

90. The method of any one of embodiments 87-89, wherein the B cell depleting therapy comprises Inebilizumab or a derivative thereof.

91. The method of any one of embodiments 87-90, wherein the therapeutically effective amount of the B cell depleting therapy is about 300 mg.

92. A method of reducing neuromyelitis optica spectrum disorder (NMOSD)-related disability in a subject in need thereof, the method comprising administering a composition that comprises Inebilizumab or a derivative thereof to the subject in need thereof, thereby reducing the NMOSD-related disability, wherein the subject in need thereof comprises: (a) an increase in serum Neurofilament light chain (sNfL) levels over a baseline level of the subject in need thereof; or (b) an increase in sNfL levels over an otherwise comparable control subject.

93. The method of embodiment 92, further comprising identifying the subject in need thereof.

94. A method of reducing neuromyelitis optica spectrum disorder (NMOSD)-related disability in a subject diagnosed with NMOSD, the method comprising administering a composition that comprises Inebilizumab or a derivative thereof to the subject diagnosed with NMODS, wherein the subject diagnosed with NMODS comprises: (a) an increase in serum Neurofilament light chain (sNfL) level over a baseline level of the subject diagnosed with NMODS; or (b) an increase in sNfL level over an otherwise comparable control subject.

95. A method of treating neuromyelitis optica spectrum disorder (NMOSD) in a subject in need thereof, the method comprising: (a) identifying a subject in need thereof at increased risk for NMOSD-related disability as determined by: (i) an increased serum Neurofilament light chain (sNfL) level over a baseline level of the subject in need thereof; or (ii) an increased sNfL level over an otherwise comparable control subject; and (b) administering a composition that comprises Inebilizumab or a derivative thereof to the subject identified in (a), thereby treating the NMODS.

96. A method of treating neuromyelitis optica spectrum disorder (NMOSD) in a subject in need thereof, the method comprising administering a composition that comprises Inebilizumab or a derivative thereof to the subject in need thereof, thereby treating the NMODS, wherein the subject in need thereof comprises: an increased serum Neurofilament light chain (sNfL) level over a baseline level of the subject in need thereof; or (b) an increased sNfL level over an otherwise comparable control subject.

97. The method of any one of embodiments 92-96, wherein the subject in need thereof comprises about 1.0, about 1.1, about 1.2, about 1.3, about 1.4, about 1.5, about 1.6, about 1.7, about 1.8, about 1.9, about 2.0 or greater fold change in serum Nfl over a baseline level.

98. The method of any one of embodiments 92-97, wherein the subject in need thereof has a sGFAP concentration of about 160 pg/mL, about 165 pg/mL, about 166 pg/mL, about 167 pg/mL, about 168 pg/mL, about 169 pg/mL, about 170 pg/mL, about 171 pg/mL, about 172 pg/mL, or about 173 pg/mL or greater.

99. A method of treating a subject suspected of having neuromyelitis optica spectrum disorder (NMOSD), the method comprising administering a composition that comprises Inebilizumab or a derivative thereof to the subject, wherein the subject comprises one or more NMODS-related symptoms and at least one of: (a) an increase in serum Neurofilament light chain (sNfL) level over a baseline level of the subject; or (b) an increase in sNfL level over an otherwise comparable control subject.

100. A method of treating a subject suspected of having neuromyelitis optica spectrum disorder (NMOSD), the method comprising: (a) identifying a subject as having one or more NMOSD-related symptoms; (b) determining if the subject identified in (a) is at increased risk for NMOSD-related disability as determined by (i) an increase in serum Neurofilament light chain (sNfL) level over a baseline level of the subject identified in (a); or (ii) an increase in sNfL levels over an otherwise comparable control subject; and (c) administering a composition that comprises Inebilizumab or a derivative thereof to the subject determined to be at increased risk for NMOSD-related disability from (b).

101. A method of treating neuromyelitis optica spectrum disorder (NMOSD) in a subject in need thereof, the method comprising administering a therapeutic in an amount effective to treat the NMODS in the subject in need thereof, wherein the subject in need thereof comprises: (a) an increase in serum Neurofilament light chain (sNfL) level over a baseline level of the subject in need thereof; or (b) an increase in sNfL level over an otherwise comparable control subject.

102. A method of treating a subject suspected of having neuromyelitis optica spectrum disorder (NMOSD), the method comprising administering a therapeutic to the subject suspected of having NMOSD, wherein the subject suspected of having NMOSD comprises one or more NMODS-related symptoms and at least one of: (a) an increase in serum Neurofilament light chain (sNfL) level over a baseline level of the subject suspected of having NMOSD; or (b) an increase in sNfL level over an otherwise comparable control subject.

103. The method of embodiment 101 or embodiment 102, wherein the therapeutic comprises one or more of Eculizumab, Satralizumab, Ublituximab, Ravulizumab, Rituximab, Azathioprine, Mycophenolate Mofetil, or a low dose corticosteroid.

### EXAMPLES

### Example 1: Design of a Clinical Trial to Investigate VIB551 as a Treatment for NMOSD, and that Identified a Relationship Between sGFAP Levels and NMOSD Disease Activity

Clinical Trial to Investigate VIB551 as a Treatment for NMOSD. VIB551 was investigated as a treatment for NMOSD in a clinical trial referred to as the “N-MOmentum study”. Full details of the N-MOmentum study have been published (Cree B., et. al, Lancet, 2019:394(10206):1352-1363). Briefly, the N-MOmentum study was an international, multicenter, randomized, double-blind, placebo-controlled phase 2/3 trial with an open-label extension phase (ClinicalTrials.gov, NCT02200770). Adults (aged ≥18 years) with a diagnosis of NMOSD, an Expanded Disability Status Scale score of ≤8.0, and a history of either at least one attack in the previous year or at least two attacks in the previous 2 years requiring rescue therapy were eligible for inclusion. Both AQP4-IgG-seropositive and AQP4-IgG-seronegative subjects were eligible. Seronegative subjects also had to meet diagnostic criteria for neuromyelitis optica described by Wingerchuk and colleagues (Wingerchuk D M, et. al., Neurology, 2006:66(10):1485-1489) and had to be confirmed by an independent panel.

Eligible subjects were randomized (3:1) to intravenous VIB551 300 mg or placebo administered on days 1 and 15 of the randomized controlled period (RCP). All study participants received oral corticosteroids (prednisone 20 mg per day or equivalent) during the initial treatment period (days 1-14, tapering to day 21) to minimize risk of an attack immediately after the first VIB551 dose; other immunosuppressant use was not permitted during the RCP. Subjects continued in the RCP for up to 28 weeks or until the occurrence of an adjudicated attack, with study visits conducted on days 1, 8, 15, 29, 57, 85, 113, 155, and 197 of the RCP. After completing the RCP or following an adjudicated attack, subjects could enter an optional open-label VIB551 treatment period of at least 1 year.

Of the 215 study participants, most were women (194/215 [90%]), and approximately half were white (110/215 [51%]). 164 were treated with VIB551 and 51 received placebo. Demographics of the participants were largely similar between the VIB551- and placebo-treated groups. See Table 1.

TABLE 1
Participant Demographics and Characteristics at Baseline (Intent-to-Treat sGFAP Analysis set.

| | Placebo | | VIB551 | | RRMS | | Healthy Donors | |
|---|---|---|---|---|---|---|---|---|
| Demographic/Characteristic | (n = 51) | | (n =164) | | (n = 23) | | (n = 25) | |
| Age, years | | | | | | | | |
| Mean (SD) | 43.4 | (14.0) | 43.0 | (11.2) | 45.3 | (12.3) | 43.8 | (8.3) |
| Median (range) | 43.0 | (20-74) | 43.0 | (18-73) | 44.0 | (21-63) | 43.5 | (29-60) |
| Sex | | | | | | | | |
| Women | 45 | (88.2) | 149 | (90.9) | 14 | (60.9) | 11 | (44.0) |
| Race | | | | | | | | |
| American Indian or Alaskan | 5 | (9.8) | 13 | (7.9) | 0 | (0.0) | 0 | (0.0) |
| Native | | | | | | | | |
| Asian | 7 | (13.7) | 38 | (23.2) | 0 | (0.0) | 0 | (0.0) |
| Black or African American | 5 | (9.8) | 15 | (9.1) | 3 | (13.0) | 2 | (8.0) |
| White | 26 | (51.0) | 84 | (51.2) | 18 | (78.3) | 22 | (88.0) |
| Other | 8 | (15.7) | 13 | (7.9) | 1 | (4.3) | 0 | (0.0) |
| Multiple categories checked | 0 | (0.0) | 1 | (0.6) | 1 | (4.3) | 0 | (0.0) |
| Ethnicity | | | | | | | | |
| Hispanic or Latino | 14 | (27.5) | 27 | (16.5) | 1 | (4.3) | 2 | (8.0) |
| Disease duration, years | | | | | | | | |
| Mean (SD) | 2.77 | (3.54) | 2.36 | (3.32) | 8.9 | (9.9) | N/A | |
| Median (range) | 1.25 | (0.2-16.9) | 1.06 | (0.1-22.2) | 5.3 | (0.2-36.0) | N/A | |
| Baseline EDSS score | | | | | | | | |
| Mean (SD) | 4.16 | (1.71) | 3.80 | (1.82) | 3.9 | (1.7) | N/A | |
| Median (range) | 4.0 | (1.0-8.0) | 3.5 | (0.0-8.0) | 4.0 | (0.0-6.5) | N/A | |
| Serostatus | | | | | | | | |

| | | | | | | |
|---|---|---|---|---|---|---|
| AQP4-IgG seropositive | 47 | (92.2) | 151 | (92.1) | N/A | N/A |
| AQP4-IgG seronegative | 3 | (5.8) | 7 | (4.3) | N/A | N/A |
| MOG-IgG seropositive | 1 | (2.0) | 6 | (3.7) | N/A | N/A |

Data are n (%) unless stated otherwise. Race was self-reported by participants.
AQP4-IgG, aquaporin-4-immunoglobulin G; EDSS, Expanded Disability Status Scale; ITT, intent-to-treat; N/A, not applicable; MOG, myelin oligodendrocyte glycoprotein; RRMS, relapsing-remitting multiple sclerosis; SD, standard deviation.

The primary endpoint was time to the onset of an adjudicated NMOSD attack during the RCP. An attack was defined as the presence of a new symptom(s) or worsening of an existing symptom(s) related to NMOSD that met at least one of the protocol-defined criteria for an attack upon neurological evaluation. Study investigators evaluated potential attacks within 72 hours. An independent committee of three expert physicians (two neurologists and one neuro-ophthalmologist) adjudicated attacks within 17 days of the attack assessment visit.

NMOSD attack severity was graded according to the opticospinal impairment scale (OPSIS) which characterizes attacks as minor or major on the basis of changes in domain-specific scores for neurological function at a subject's attack assessment visit relative to the subject's previous assessment. Attack recovery was graded according to change in the domain-specific scores at the subject's follow-up visit relative to the subject's visit at the attack assessment visit.

Domain—, i.e., optic neuritis-, myelitis- and brain-, specific sub scores for neurological function were assigned according to the descriptions provided in Table 2.

TABLE 2
Subscale Scores by Domain

| | Subscale | |
|---|---|---|
| Domain | Score | Description |
| Optic | 1 | Scotoma but VA ≥50 characters |
| neuritis | 2 | VA ≥35-49 characters |
| | 3 | VA ≥20-34 characters |
| | 4 | VA ≥1-19 characters |
| | 5 | Counting fingers only |
| | 6 | Light perception only |
| | 7 | No light perception |
| Myelitis | 0 | Normal |
| | 1 | Abnormal signs (hyperreflexia, Babinski sign) without weakness |
| | 2 | Mild weakness (MRC grade 5− or 4+) in affected limb(s) |
| | 3 | Moderate weakness (grade 3 or 4) in 1 or 2 UMN muscles in affected |
| | | limb(s) |
| | 4 | Moderate weakness (grade 3 or 4) in 3 UMN muscles in affected limb(s) |
| | 5 | Severe weakness (grade 2) in 1 or more muscles in affected limb(s) |
| | 6 | Some plegic (grade 0 or 1) muscles in 1 or more limbs |
| | 7 | Plegia (grade 0 or 1) of all muscles in 1 or more limbs |
| Brain | 0 | Normal |
| | 1 | Drowsiness or mood changes only |
| | 2 | Mild confusion/disorientation (able to manage all self-care functions); mild |
| | | focal impairment (mild aphasia, apraxia, agnosia, anorexia, or drowsiness) |
| | 3 | Moderate confusion/disorientation (able to manage some self-care |
| | | functions); moderate focal impairment (moderate aphasia, apraxia, agnosia, |
| | | anorexia, or drowsiness) |
| | 4 | Severe confusion/disorientation (unable to manage self-care functions); |
| | | severe focal impairment (aphasia such that is unable to comprehend simple |
| | | one-step commands or speak 5-word sentences; severe apraxia, agnosia, |
| | | anorexia, or drowsiness) |
| | 5 | Stupor or coma |
| Brainstem | 0 | Normal |
| | 1 | Signs only (unsustained nystagmus, impaired saccadic pursuit, ocular |
| | | dysmetria, mild facial weakness, or sensory loss) |
| | 2 | Sustained conjugate nystagmus, incomplete INO, moderate facial weakness |
| | | or sensory loss, or other mild disability; mild nausea and vomiting for 48 |
| | | hours or longer without other explanation with vomiting not more than 3 |
| | | times per day; intractable hiccups occurring more than 20 times per hour less |
| | | than 6 hours per day |
| | 3 | Dyconjugate nystagmus (INO) or severe extraocular weakness, loss of facial |
| | | sensation or facial paralysis (unilateral or bilateral), moderate dysarthria or |
| | | dysphagia; moderate nausea and vomiting lasting 48 hours or longer without |
| | | other explanation with vomiting between 3 and 7 times per day; intractable |
| | | hiccups occurring more than 20 times per hour for 6-12 hours per day |
| | 4 | Severe dysarthria or dysphagia, almost complete ophthalmoplegia, or other |
| | | severe disability of a cranial nerve/nerves; severe nausea and vomiting |
| | | lasting 48 hours or longer without other explanation with vomiting occurring |
| | | more than 7 times per day; intractable hiccups occurring more than 20 times |
| | | per hour for more than 12 hours per day |
| | 5 | Inability to swallow or speak because of bulbar dysfunction; respiratory |
| | | failure requiring intubation because of brainstem lesion |

INO, internuclear ophthalmoplegia; MRC, medical research council; UMN, upper motor neuron; VA, visual acuity.

Change(s) in domain-specific sub scores for neurological function resulting in the grading of a subject's attack severity as “major” or “minor” are shown in Table 3.

TABLE 3
Change in Subscale Domain Score Corresponding
to NMOSD Attack Severity Grade

| | Subscale score at | Subscale score at | |
|---|---|---|---|
| Domain | pre-attack visit | time of attack | Severity |
| Optic neuritis, | <2 | <3 | Minor |
| myelitis, | | ≥3 | Major |
| brainstem | ≥2 | Increase by 1 point | Minor |
| | | Increase by ≥2 points | Major |
| Brain | Not applicable | Increase by 1 point | Minor |
| | | Increase by ≥2 points | Major |

Change(s) in domain-specific sub scores for neurological function resulting in the grading of a subject's attack recovery as “major” or “minor” are shown in Table 4.

TABLE 4
Change in Subscale Domain Score Corresponding
to NMOSD Attack Recovery Grade

| | Subscale score at | Improvement at | |
|---|---|---|---|
| Domain | time of attack | follow-up visit | Recovery |
| Optic neuritis, | Any score | ≤2 | Minor |
| myelitis, brainstem | ≥3 | >2 | Major |
| Brain | Any score | 1 | Minor |
| | ≥2 | >1 | Major |

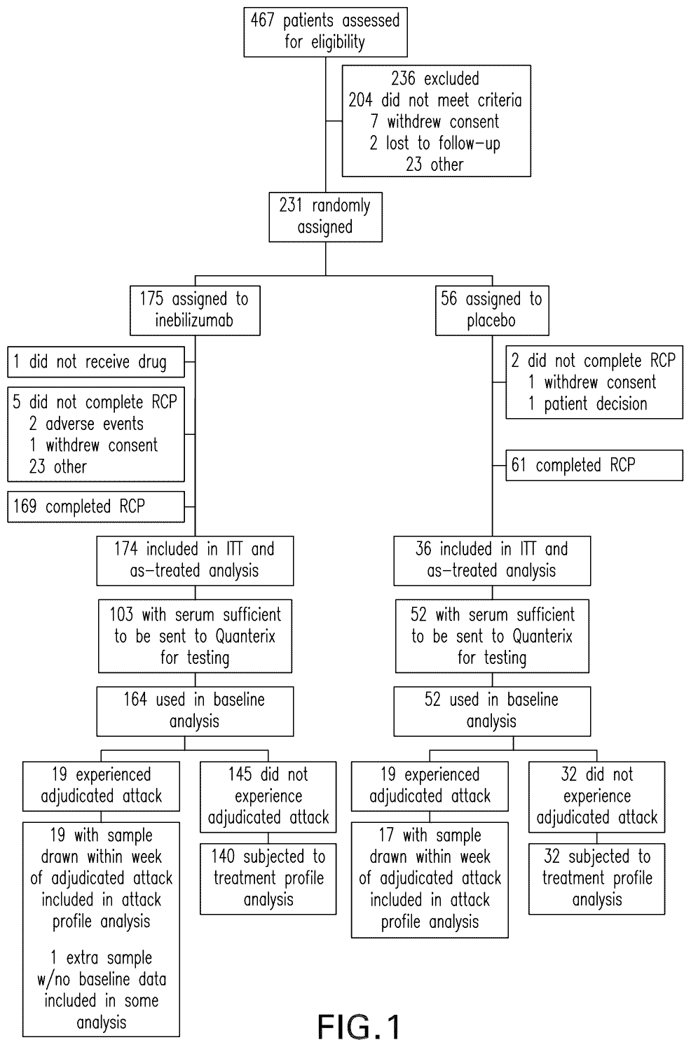

Determination of sGFAP Levels in the N-MOmentum Study. A predefined, exploratory outcome of the N-Momentum study was to compare the effect of VIB551 versus placebo on sGFAP concentrations and the potential of sGFAP as a biomarker in subjects with NMOSD. sGFAP concentrations were analyzed from blood samples collected from participants during RCP study visits at baseline (day 1), day 15, day 29, day 57, day 85, day 113, day 155, and day 197, and during any assessment visit for new or worsening NMOSD symptoms. A Consolidated Standards of Reporting (CONSORT) diagram for the N-MOmentum study participants is provided in FIG. 1. Two reference cohorts of individuals without NMOSD were also assessed: healthy donors (n=25) and subjects with RRMS (n=23). Table 1. For the reference cohorts, blood was collected at the single baseline visit. As validated in serum samples from subjects with MS or traumatic brain injury (Abdelhak A, et. al., Sci Rep. 2018; 8(1):14798; Czeiter E, et. al., EBioMedicine. 2020; 56:102785), sGFAP concentration was determined by a single molecule array (Simoa) technology, using the Quanterix Simoa GFAP assay (Quanterix Corporation, Lexington, MA, USA) with samples run according to manufacturer's instructions.

Differences in sGFAP concentration between groups were evaluated for statistical significance using the Mann-Whitney U test and the Cochran-Armitage test. Changes in sGFAP concentration from baseline were assessed using the Wilcoxon signed-rank test.

A total of 215 participants from the N-MOmentum study (including 198 AQP4-IgG seropositive and 17 AQP4 seronegative) provided 1260 serial and NMOSD attack-related samples for sGFAP analysis.

### Example 2: Baseline sGFAP Levels are Elevated in NMOSD Subjects Compared to RRMS Subjects and Healthy Donors

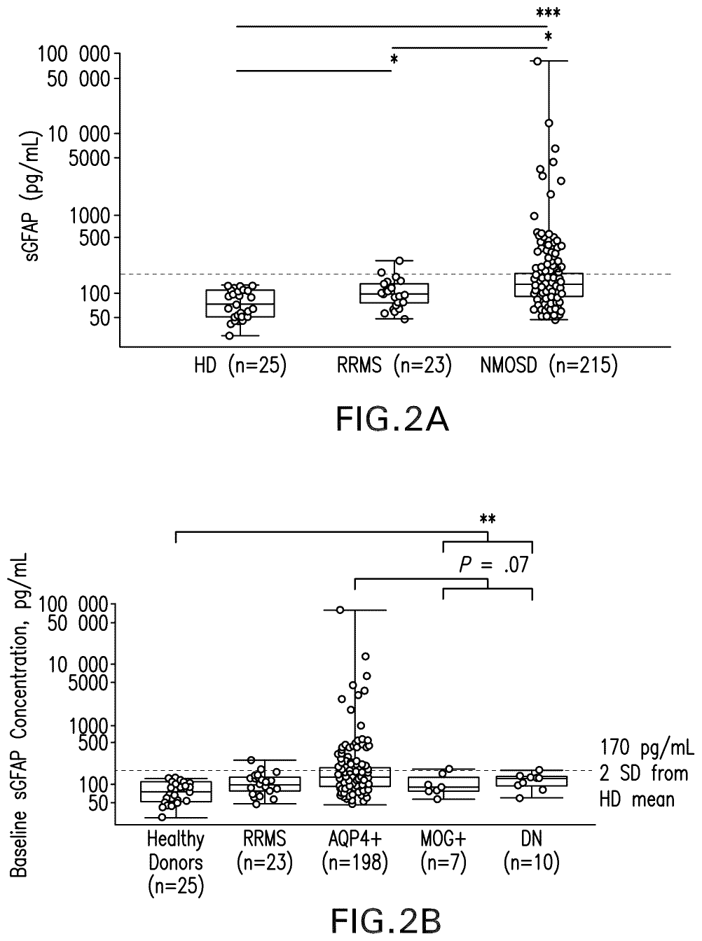

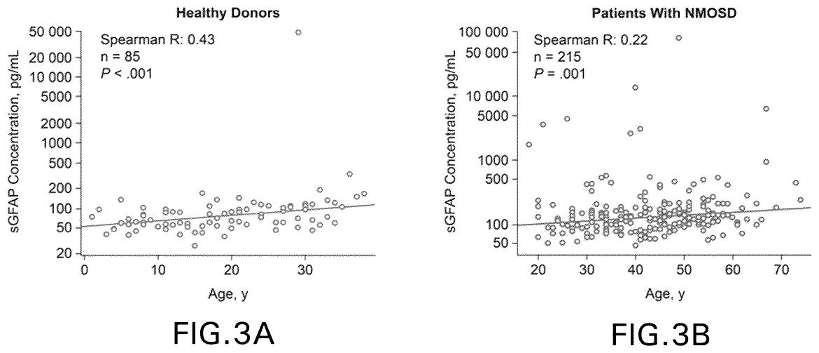

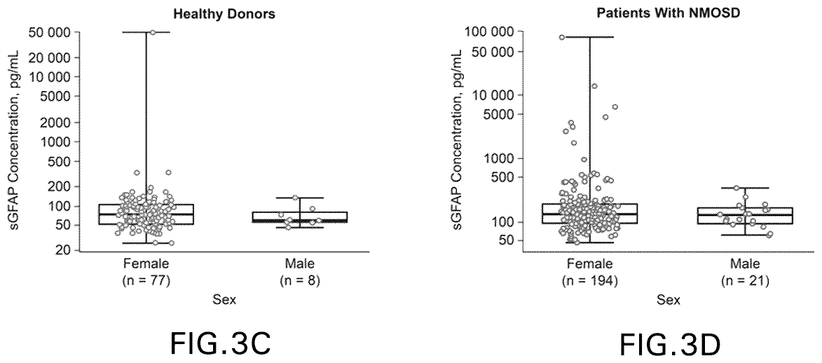

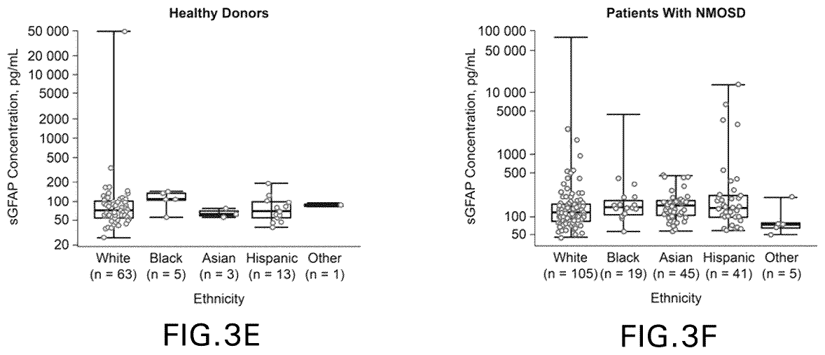

Elevated sGFAP levels were observed in significantly more participants with NMOSD at study baseline (62/215 [29%]) than in individuals with RRMS (2/23 [9%]) or healthy donors (2/85 [2.4%]); P<0.05 and P<0.001, respectively (FIG. 2A). Median (interquartile range [IQR]) sGFAP concentration was 128.3 (92.0-181.2) pg/mL for participants with NMOSD compared with 71.3 (55.6-102.2) pg/mL for age- and sex-matched healthy donors and 97.5 (76.5-131.4) pg/mL for individuals with RRMS. Two AQP4-IgG-seronegative participants (one who was MOG-IgG seropositive) had elevated baseline sGFAP levels (FIG. 2B). For both subjects with NMOSD and healthy controls, a modest but clear age-dependent increase of sGFAP was observed (FIG. 3A-FIG. 3F), whereas gender or ethnicity had no effect

Elevated sGFAP concentrations were defined by being ≥2 standard deviations (SD) above the healthy donor mean concentration (≥170 pg/mL) according to established laboratory procedures (Marshall W J, Bangert S K. Clinical Biochemistry: metabolic and clinical aspects. 2nd edition ed: Churchill Livingstone; 2008).

### Example 3: NMOSD Subjects with Elevated Baseline sGFAP Levels are at Increased Risk of an NMOSD Attack

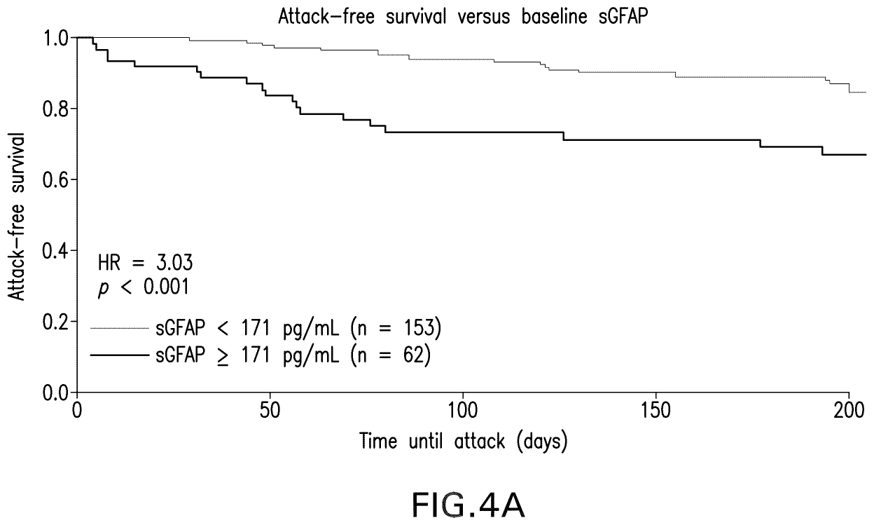

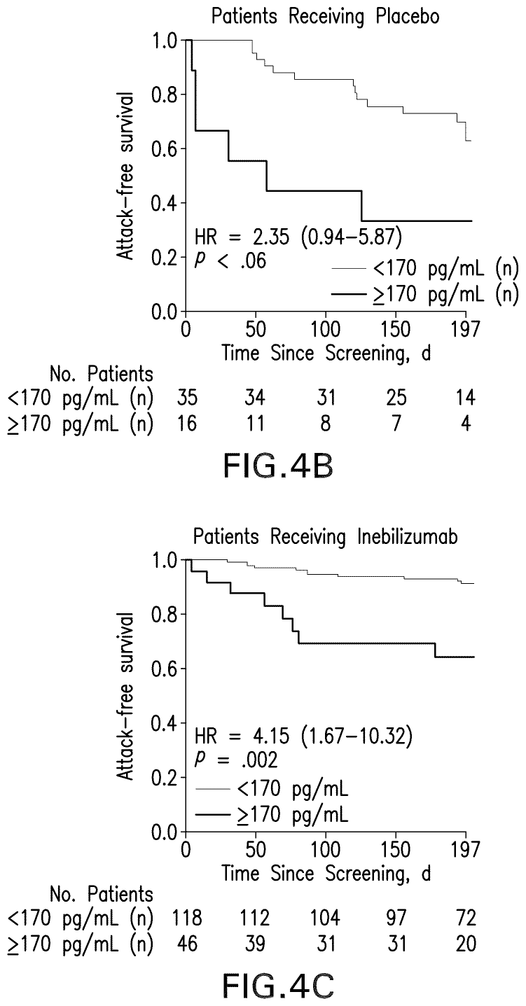

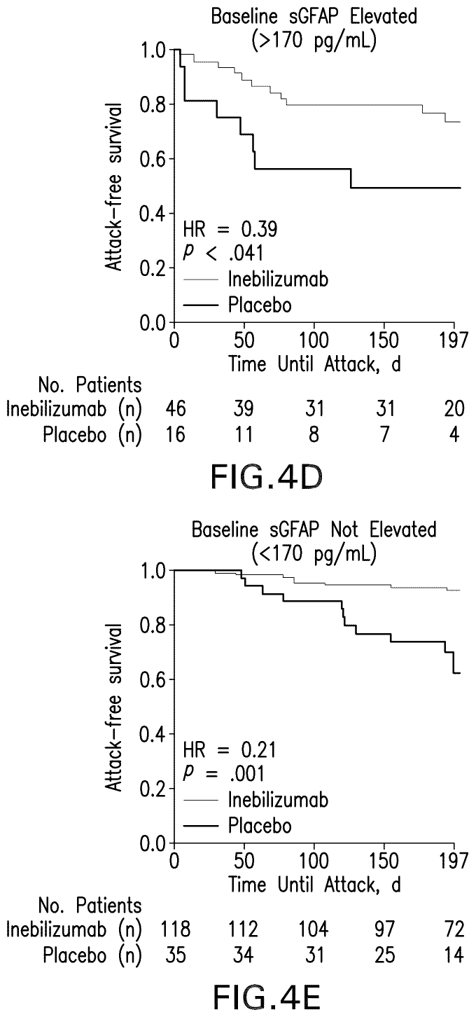

Participants with NMOSD and with an elevated baseline sGFAP concentration were at increased risk of experiencing an adjudicated NMOSD attack. Analysis of all study participants showed that 19/62 subjects (31%) with elevated sGFAP at baseline experienced an adjudicated NMOSD attack versus 19/153 subjects (12%) without elevated sGFAP, equating to three times the risk of an attack during the RCP (HR [95% Confidence Interval [CI], 3.03 [1.57-6.10]; P=0.001; FIG. 4A). A similar pattern was observed for both the placebo and the Inebilizumab (VIB551) group (for placebo: HR [95% CI], 2.35 [0.94-5.87]; P=0.06, FIG. 4B; for Inebilizumab (VIB551): HR [95% CI], 4.15 [1.67-10.32]; P=0.002; FIG. 4C). Increased baseline sGFAP concentrations did not correlate with either most recent pre-study NMOSD attacks or with age/baseline EDSS score (Tables 5 and 6).

TABLE 5
Results from Hazard Regression of Baseline sGFAP vs
Time to Attack Adjusted for Baseline Covariates

| Variable name | HR (95% CI) | P value |
|---|---|---|
| Baseline sGFAP concentration ≥170 pg/mL | 3.09 (1.57-6.10) | .001 |
| Attack within 60 days before RCP start | 0.44 (0.10-1.91) | .27 |
| Age, y | 0.98 (0.96-1.02) | .21 |
| Baseline EDSS score | 1.09 (0.91-1.31) | .34 |

TABLE 6
Results from Hazard Regression of Baseline sGFAP vs
Time to Attack Adjusted for Baseline Covariates

| Variable name | HR (95% CI) | P value |
|---|---|---|
| Baseline sGFAP concentration ≥170 pg/mL | 2.99 (1.51-5.89) | .002 |
| Attack within 90 days before RCP start | 0.67 (0.31-1.47) | .32 |
| Age, y | 0.98 (0.96-1.02) | .21 |
| Baseline EDSS score | 1.09 (0.91-1.31) | .35 |

### Example 4: VIB551 Reduces Risk of NMOSD-Related Attacks in NMOSD Subjects, Regardless of Baseline sGFAP Level

Further analysis of subjects according to treatment group showed that VIB551 therapy was associated with a decreased risk of adjudicated attack in subjects with NMOSD. In subjects with elevated baseline sGFAP concentration, VIB551 reduced the risk of an adjudicated attack by 61% compared with placebo (HR [95% CI] 0.39 [0.15-0.96]; P=0.041; FIG. 4D). In subjects without elevated baseline sGFAP levels, the risk of an adjudicated attack was reduced by 79% with VIB551 compared with placebo (HR [95% CI] 0.21 [0.08-0.51]; P<0.001; FIG. 4E).

### Example 5: sGFAP Levels Increase within One Week of an NMOSD-Related Attack

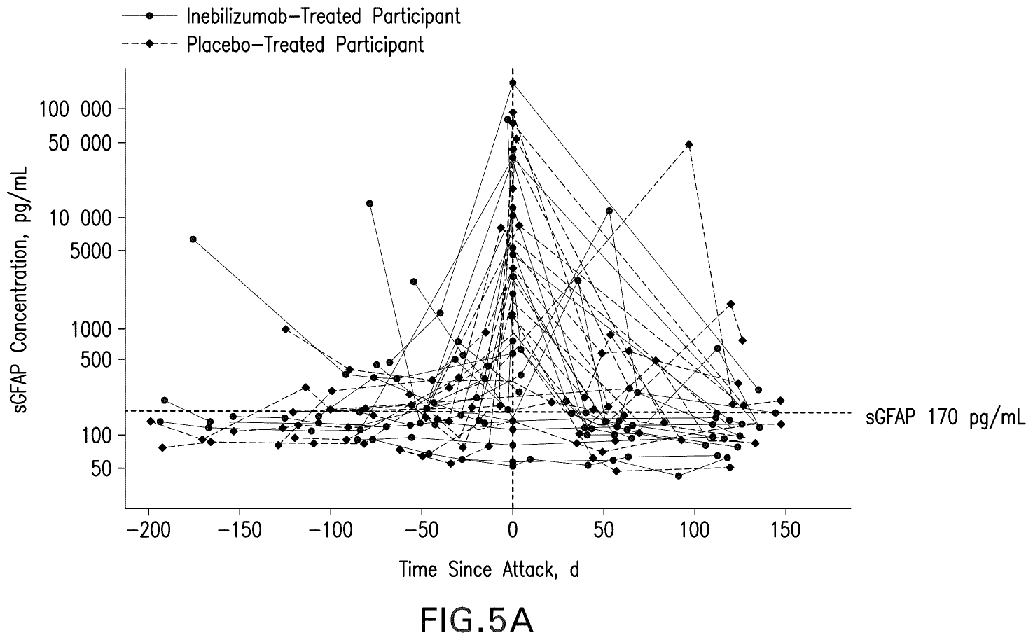

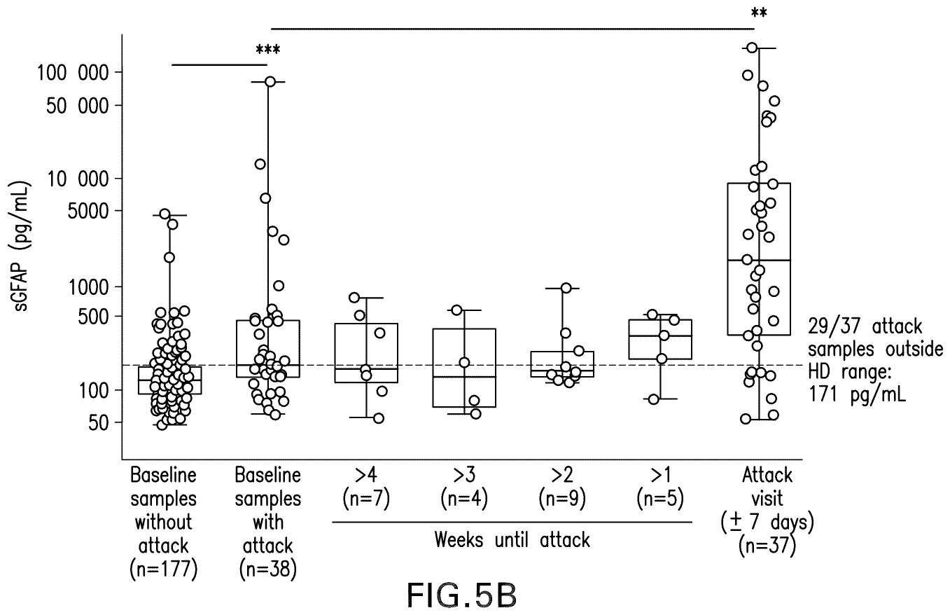

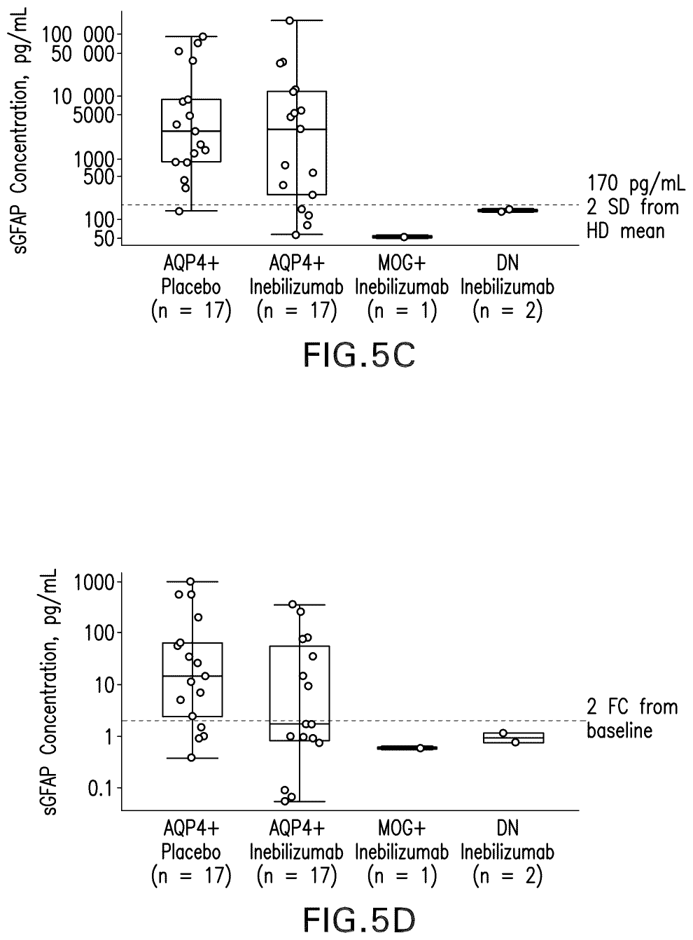

Increases in sGFAP levels were observed in subjects during an adjudicated NMOSD attack (within 1 week, either before or after, of the attack). A significant increase in sGFAP concentration from baseline was observed for subjects who had an adjudicated NMOSD attack (median [IQR]: baseline, 168.4 [128.9-449.7] pg/mL; attack, 2160.1 [302.7-9455.0] pg/mL; P=0.015; FIG. 5A). An elevated sGFAP concentration was observed in 29 (out of 37) attack samples (78%) compared with 19 (out of 38) samples (50%) at baseline. By contrast, sGFAP levels in samples taken more than 1 week before an attack were similar to those at baseline (FIG. 5B). Attacks in MOG-IgG-seropositive or double seronegative participants were too infrequent to meaningfully analyze for change in sGFAP (FIG. 5C-FIG. 5D).

### Example 6: Degree of Increase in sGFAP Concentration Leading Up to an NMOSD-Related Attack Predicts Attack Severity

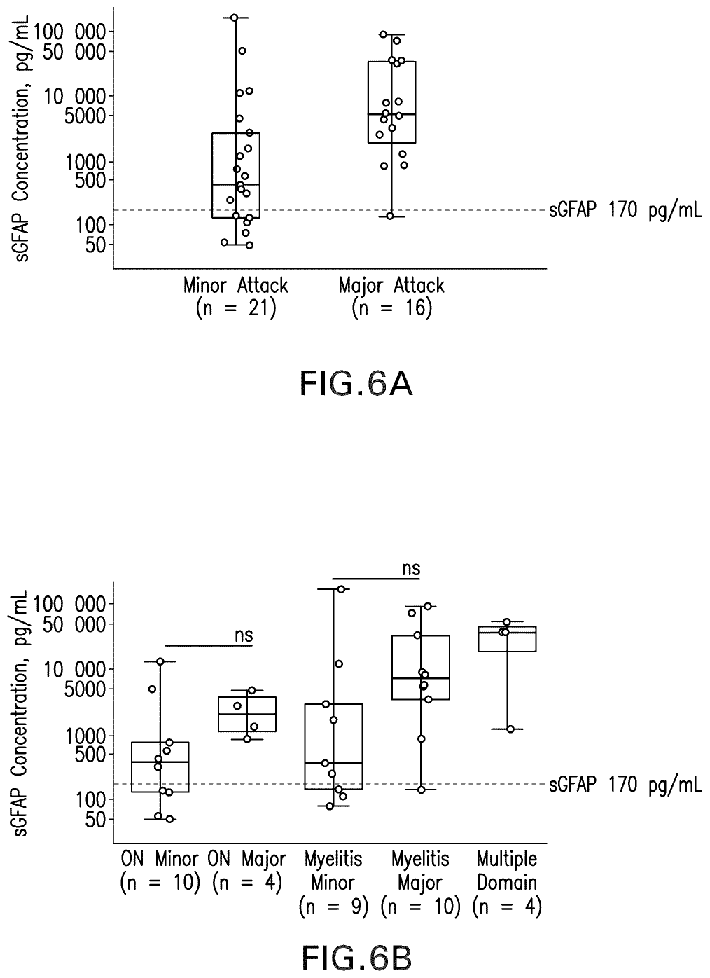

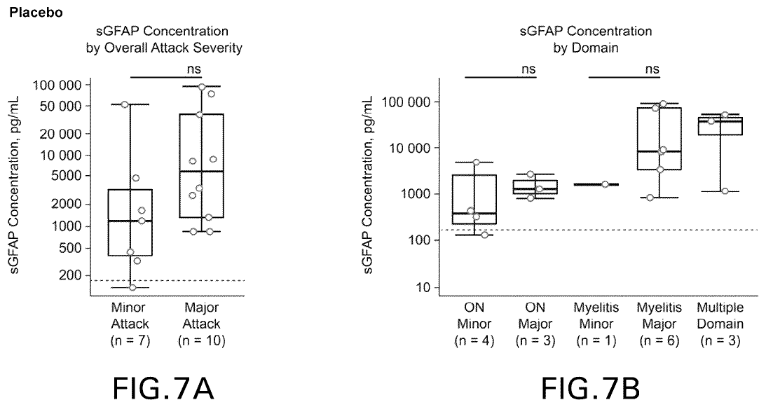

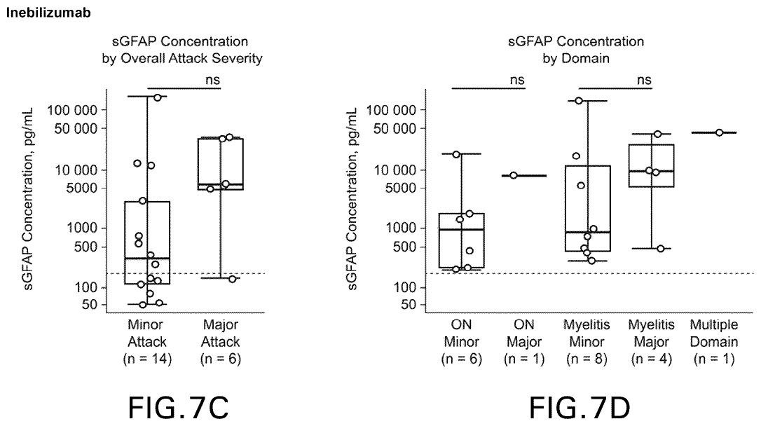

Elevated sGFAP concentration was also associated with the severity of the adjudicated NMOSD attack. sGFAP concentration during an attack was significantly higher in subjects who had major adjudicated attacks than in those who had minor adjudicated attacks (median [IQR]: major attacks, 34.32 [8.72-107.53] pg/mL; minor attacks. 1.06 [0.85-7.43] pg/mL; P=0.023; FIG. 6A). In addition, sGFAP concentrations tended to be higher during major adjudicated attacks compared with minor adjudicated attacks across all domains, including attacks that only affected the optic nerve (FIG. 6B). This trend was consistent in both the VIB551 and placebo treatment groups (FIG. 7A-FIG. 7D).

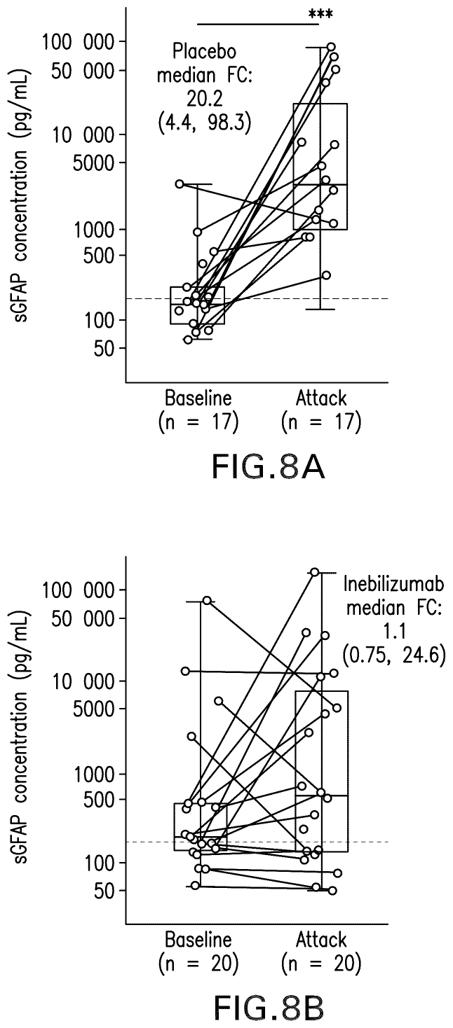

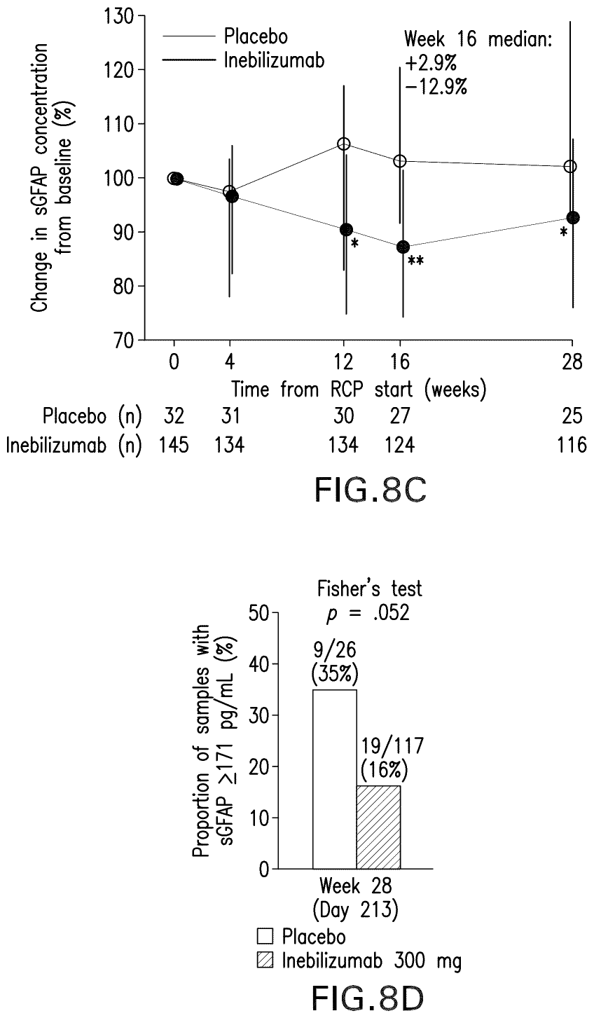

Analysis of participants according to study treatment showed that sGFAP concentration during an adjudicated NMOSD attack was not significantly increased in the VIB551 group, and was significantly lower in VIB551-treated subjects than in those receiving placebo (median [IQR]: VIB551 [n=20], 653.0 [139.0-7227.8] pg/mL; placebo [n=17], 3056.1 [1091.5-858.5] pg/mL; P=0.048). In participants receiving placebo, sGFAP concentration during an attack showed a median fold change of 20.2 from baseline (P=0.001; FIG. 8A) while in participants treated with VIB551 no relevant increase in sGFAP concentration was observed (median fold change, 1.1; P=0.31; FIG. 8B). During the adjudicated attacks, seven (out of 20) samples from VIB551-treated subjects did not have elevated sGFAP concentrations compared with one sample (out of 17) from subjects receiving placebo.

### Example 7: VIB551 Decreases sGFAP Levels in Attack-Free NMOSD Subjects

For subjects who did not experience an adjudicated NMOSD attack during the RCP, sGFAP concentrations decreased with VIB551 treatment after week 4. The reduction from baseline was statistically significant from week 16 (median [IQR] reduction of 12.9 [−25.6, −1.6]%) to the end of the RCP (P<0.05; FIG. 8C). Conversely, there was no significant change in sGFAP in subjects receiving placebo without attacks at any point in the RCP (FIG. 8C). By the end of the RCP (week 28), 35% (n/N=9/26) of subjects within the placebo group had elevated sGFAP concentrations compared with 16% (n/N=19/117) of subjects within the VIB551 group.

### Example 8: Even in NMOSD Subjects that Did not Experience an NMOSD-Related Attack, an Increase in sGFAP Concentration Indicated an Increase in NMOSD Disease Activity

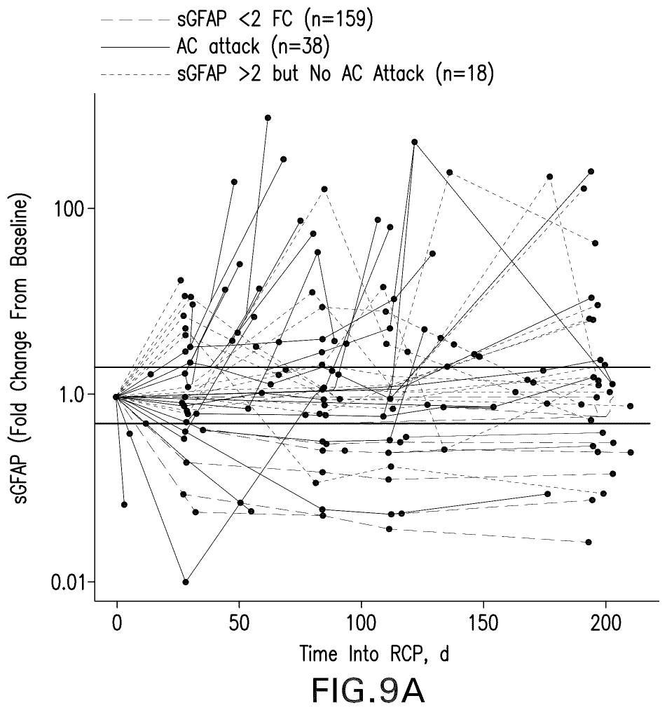

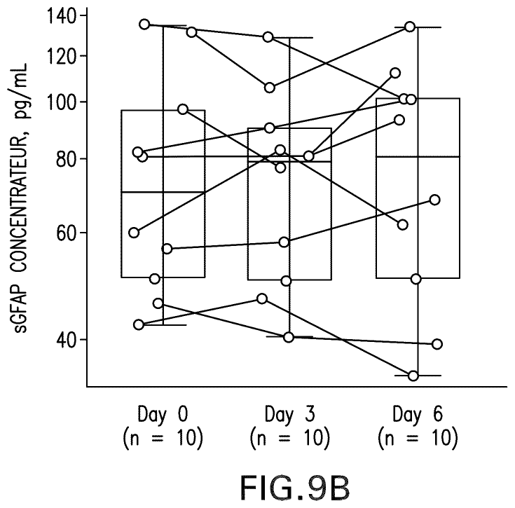

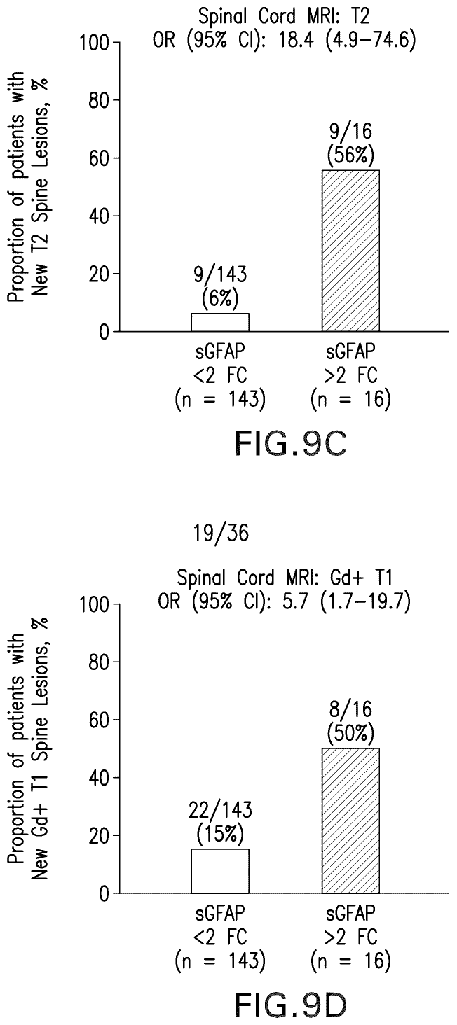

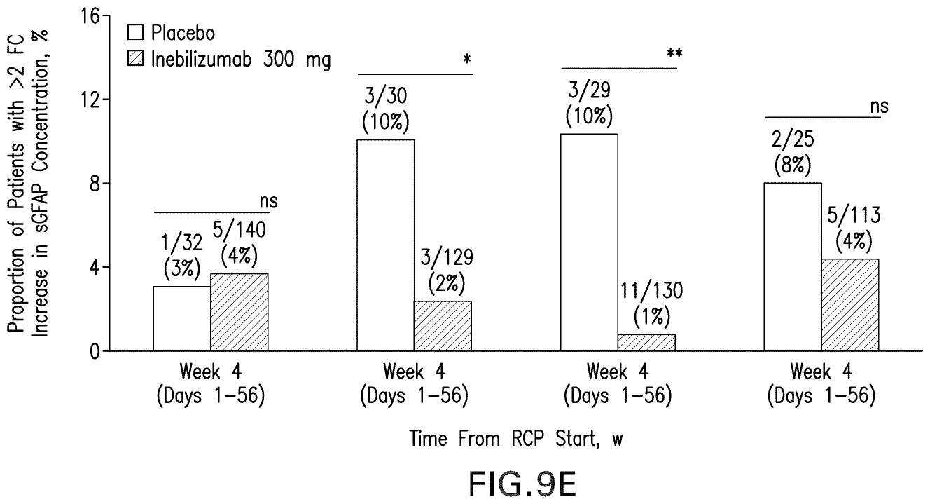

Among the 161 participants without an attack, 18 (11.2%) displayed an increase greater than twofold in sGFAP concentration in at least one sample draw. These increases from baseline were comparable to the range observed in subjects with attacks (FIG. 9A) and well outside the variation observed in longitudinal draws from healthy donors (FIG. 9B). Five of these 18 participants (28%) with an elevation in sGFAP reported neurological symptoms that were rated as attacks by the treating investigators but not confirmed by the adjudication committee (AC). Of note, for the total cohort of these 18 participants, increased rate of adverse events was observed in temporal vicinity to sampling (Table 7).

TABLE 7
Overview of Adverse Event and Severe Adverse Events in Attack-Free
Subjects with sGFAP Increases

| | Subjects | Subjects | No | | No | | | |
|---|---|---|---|---|---|---|---|---|
| | with AE | with AE | SGFAP | SGFAP | sGFAP | SGFAP | | |
| | and no | and | increase, | increase | increase, | increase, | | |
| | SGFAP | SGFAP | median | median | mean AI | mean, | RR | NB |
| | increase, | increase | AI count | AE count | count | AI count | (95% | regression |
| Field | # (%) | # (%) | (Q1, Q3) | (Q1, Q3) | (SEM)q | (SEM) | CI) | P value |
| Any AE in RCP | 116/158 | 15/18 | 2 (0, 4) | 3 (1, 4) | 2.99 | 4.67 | 1.56 | .139 |
| | (73%) | (83%) | | | (2.66- | (3.24, | (2.81- | |
| | | | | | 3.33) | 6.09) | 0.87) | |
| Any SAB in RCP | 7/158 | 1/18 | 0 (0, 0) | 0 (0, 0) | 0.06 | 0.17 | 2.93 | .343 |
| | (4%) | (6%) | | | (0.03, | (0.00- | (26.9- | |
| | | | | | 0.08) | 0.33) | 0.32) | |

| | Samples with | Samples | | |
|---|---|---|---|---|
| | AE and | with AE | | Fischer |
| | no sGFAP | and sGFAP | | exact |
| | increase, | increase, | | test P |
| Field | # (%) | #(%) | OR (95% CI) | value |
| Any AE within 7 | 220/1015 (21.7%) | 9/23 (39%) | 2.32 (0.87-5.85) | .07 |
| days of sample | | | | |
| Any AE within 14 | 257/1015 (25.3%) | 12/23 (52%) | 3.21 (1.28-8.15) | .007 |
| days of sample | | | | |
| Any AE within 30 | 328/1015 (32.3%) | 12/23 (52%) | 2.29 (0.91-5.78) | .07 |
| days of sample | | | | |
| Any AE within 45 | 386/1015 (38.0%)) | 13/23 (57%) | 2.12 (0.85-5.45) | .08 |
| days of sample | | | | |
| SAE within 7 | 5/1015 (0.5%) | 2/23 (9%) | 19.01 (1.72-124) | .009 |
| days of sample | | | | |
| SAE within 14 | 9/1015 (0.9%) | 2/23 (9%) | 10.57 (1.05-55.8) | .02 |
| days of sample | | | | |
| SAE within 30 | 13/1015 (1.3%) | 2/23 (9%) | 7.30 (0.75-35.6) | .04 |
| days of sample | | | | |
| SAE within 45 | 16/1015 (1.6%) | 2/23 (9%) | 5.92 (0.62-27.9) | .06 |
| days of sample | | | | |

Moreover, of the subgroup of 16 attack-free subjects who had increases in sGFAP concentration greater than twofold and had spinal cord magnetic resonance imaging (MRI) scans, 9 (56%) presented with new or enlarging T2 lesions versus 9/143 subjects (6%) who neither experienced attacks nor displayed longitudinal sGFAP changes (FIG. 9C). A similar pattern was seen for gadolinium-enhancing T1 spine lesions (FIG. 9D). The proportion of participants with twofold increases in sGFAP levels was reduced by VIB551 from week 12 onwards (FIG. 9E). Thus, an increase in sGFAP levels is a signal of NMOSD-related disease activity, e.g., associated with an attack, presentation of new or enlarging MRI lesions, or increased clinical symptomology.

### Example 8: AQP4-IgG Seronegative Subjects

An autoantibody against aquaporin-4 (AQP4), a water channel expressed on astrocytes, is detected in up to 90% of subjects with NMOSD (Jarius S and Wildemann B. Nat Rev Neurol 2010; 6:383-92.) AQP4-IgG is produced by CD19 positive (CD19+) B-lineage plasmablasts, and the presence of these plasmablasts correlates with disease activity in NMO (Chihara N, et al. Proc Natl Acad Sci USA 2011; 108(9):3701-6. Kim W, et al. J Clin Neurol 2011:7(3):115-27. Greenberg B M, et al. Mult Scler 2012; 18(7):1022-6). The remaining subjects are AQP4-IgG seronegative; there are relatively few studies in this subject population. Recent studies have identified a subset of AQP4-IgG seronegative NMOSD subjects who are positive for antibodies against myelin-oligodendrocyte glycoprotein (MOG), a protein expressed on the outer surface of the myelin sheath and oligodendrocytes (Kitley J, et al. Neurology 2012; 79(12):1273-7. Mader, et al. J Neuroinflamm 2011; 8:184.)

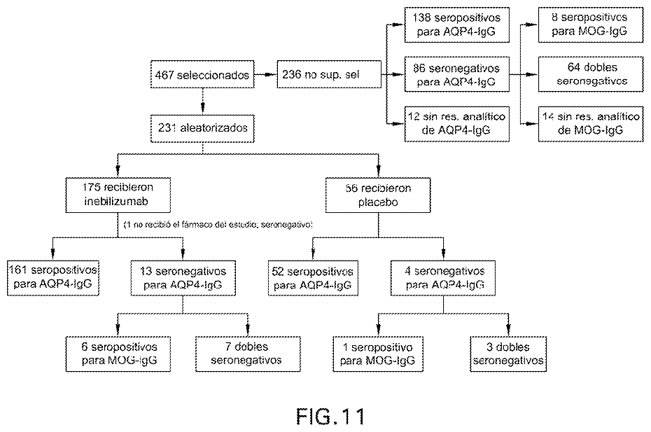

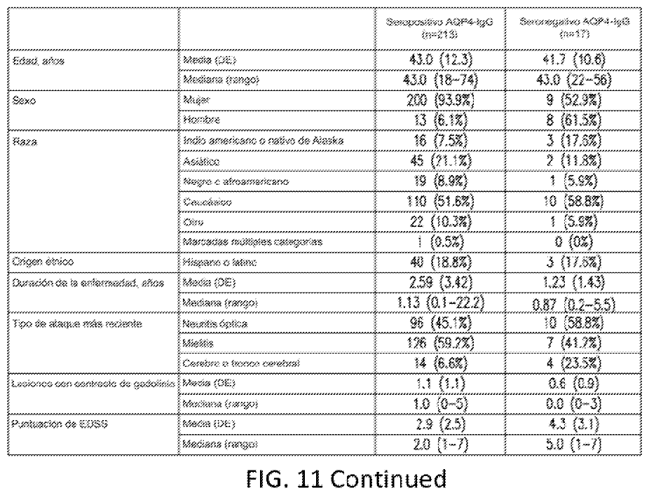

The medical histories and screening data for AQP4− subjects were assessed independently by 3 clinical experts prior to enrollment. Diagnosis using the 2006 criteria was confirmed by majority decision. Myelin oligodendrocyte glycoprotein-IgG (MOG) serology and annualized attack rates (AARs) were tested post hoc. 18/50 (36%) AQP4− subjects were eligible for randomization; 17 were randomized, 4 to placebo (1 MOG+) and 13 to Inebilizumab (6 MOG+). Owing to limited subject numbers, the on-study to the pre-study AAR were compared for treated participants to assess treatment effects. 86 prospective AQP4-IgG seronegative subjects failed screening by not meeting the 2006 NMOSD diagnosis criteria (primarily due to lacking MRI finding). FIG. 11 shows the AQP4-IgG seropositive vs. AQP4-IgG seronegative subgroup. As can be seen there is a higher proportion of male subjects and a greater baseline disability based on EDSS in the seronegative subgroup.

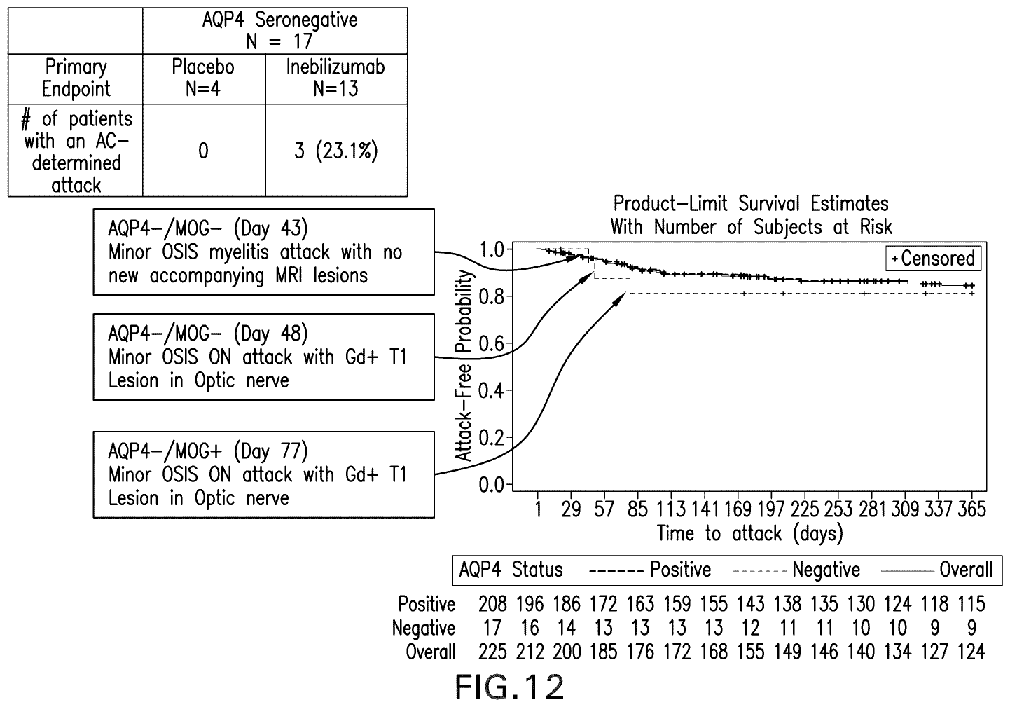

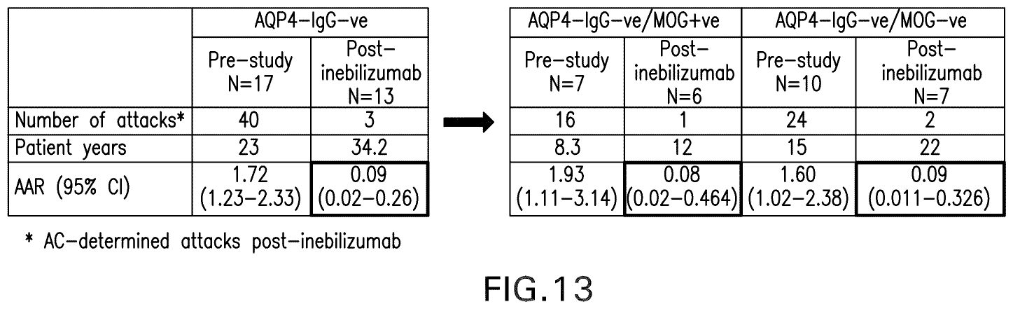

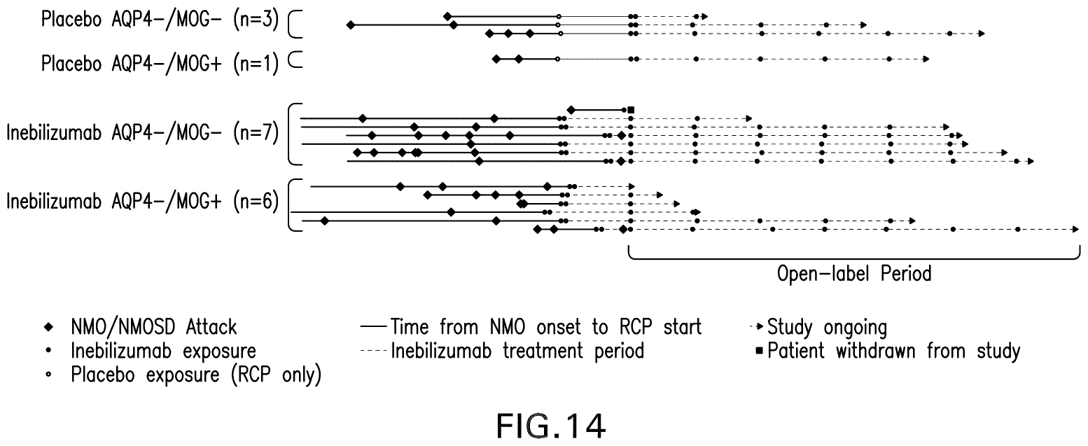

As can be seen in FIG. 12, 3/17 AQP4-IgG seronegative subjects had AC-determined NMO/NMOSD attacks during the RCP. All 3 attacks in AQP4-IgG seronegative subjects were in the Inebilizumab-treated group and occurred in the first 3 months of the RCP. There were no observed attacks in the remaining 10 Inebilizumab-treated or 4 placebo-treated AQP4-IgG seronegative subjects through the first 6 months of the OLP. FIG. 13 shows the annualized attack rates during RCP (post hoc analysis) for AQP4-IgG seronegative subjects. On-study and pre-study AARs for treated subjects were compared for treatment effects, due to the limited number of AQP4-IgG seronegative subjects who received placebo. Following Inebilizumab treatment, AARs declined in all AQP4-IgG seronegative groups by the end of the RCP. Post-Inebilizumab AARs for AQP4-IgG seronegative subjects were similar to that calculated for AQP4-IgG seropositive subjects (0.13; 95% CI: 0.09-0.18). For AQP4− participants (n=17), 40 attacks occurred in 23 subject-years of pre-study follow-up; the pre-study mean AAR (95% Confidence interval) was 1.72 (1.23-2.33). For MOG+ participants (n=7), 16 attacks occurred in 8.3 subject-years of follow-up; pre-study AAR was 1.93 (1.11-3.14). For double negative participants (n=10), 24 attacks occurred in 15 subject-years of follow-up; pre-study AAR was 1.60 (1.02-2.38). After receiving Inebilizumab, AARs declined in all groups by the end of RCP: AQP4− (n=13), 0.09 (0.02-0.26), or 3 attacks in 34.2 subject-years; MOG+ participants (n=6), 0.08 (0.002-0.464), or 1 attack in 12 subject-years; double negative participants (n=7), 0.09 (0.011-0.326), or 2 attacks in 22 subject-years. As can be seen in FIG. 14, the benefit was sustained with longer term Inebilizumab exposure. At 120 days into the open label period (OLP) where all participants receive Inebilizumab, the AAR in AQP4− participants (n=17) remained low (0.069 [0.014-0.202]). Indeed, no attacks were seen in any AQP4−, MOG+ or double seronegative subject during the OLP.

An AAR of 1.72 (95% CI: 1.23-2.33) was observed during the up to 24-month period prior to the first on-study dosing in the 17 AQP4-IgG seronegative subjects who were subsequently treated in the study. 13 AQP4-IgG seronegative subjects received Inebilizumab treatment. The AAR for the RCP was 0.09 (95% CI: 0.02-0.26), with a similar decline in ARR observed in MOG-IgG1 seropositive and MOG-IgG1 seronegative subjects. The N-MOmentum trial provided clinically important insight on the difficulty of correctly diagnosing AQP4− NMOSD and suggests that Inebilizumab may have a benefit on AAR in these subjects.

### Example 9: Serum Neurofilament Light Chain Levels (sNfL) Correlate with Attack-Related Disability in Neuromyelitis Optica

Pathogenic autoantibodies against aquaporin 4 (AQP4) in neuromyelitis optica spectrum disorder (NMOSD) cause central nervous system injury, with subsequent release of astroglial and neuronal proteins such as glial fibrillary acidic protein (GFAP), neurofilament light chain (NfL), ubiquitin carboxyl-terminal hydrolase L1 (UCH-L1) and Tau into the circulation. Serum biomarkers NfL, UCH-L1, Tau and sGFAP were measured using the single molecular array (SIMOA; Quanterix) in 1260 serial and attack-related samples from N-MOmentum participants (n=215) and healthy controls (HC; n=25).

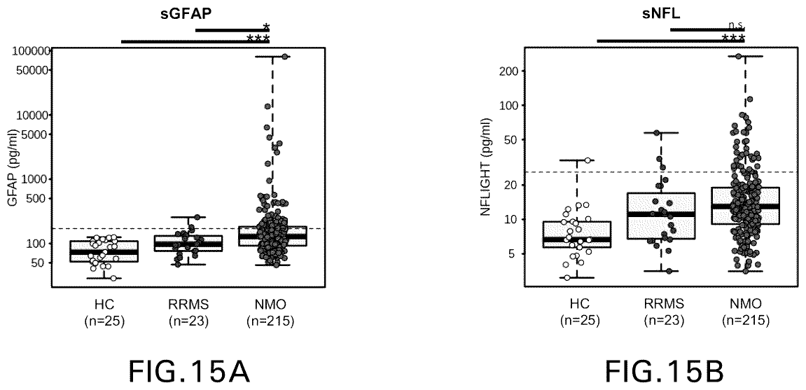

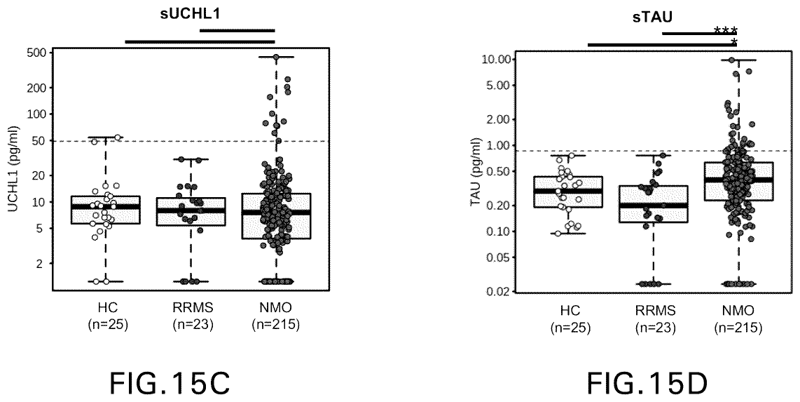

At baseline, biomarkers were elevated in subsets of subjects with NMOSD (NfL, 16%; UCH-L1, 6%; Tau, 12%; sGFAP, 29%); NfL and UCH-L1 levels correlated with sGFAP (r=0.53 [p<0.001] and 0.18 [p=0.007]). As shown in FIG. 15, Biomarker concentrations were elevated in comparison to healthy controls and subjects with relapsing-remitting multiple sclerosis (RRMS). Statistically significant increases were noted in sGFAP (29% p=3.0 e-07), sNfL (16%, p=3.4 e-6) and sTau (12%, p=0.0⁴³) in subjects with NMOSD versus healthy controls. Baseline elevations were significantly associated with increased attack risk (NfL, hazard ratio [HR] 2.5, p=0.01; UCH-L, HR 2.8, p=0.039; Tau, HR 2.6, p=0.01; sGFAP, HR 3.03, p<0.001). After controlling for baseline sGFAP in cox regressions, the other markers were not independently associated with risk of attack (all HR<2; p>0.05). In the total cohort, a greater proportion of subjects had an attack with placebo than Inebilizumab (39% versus 12%).

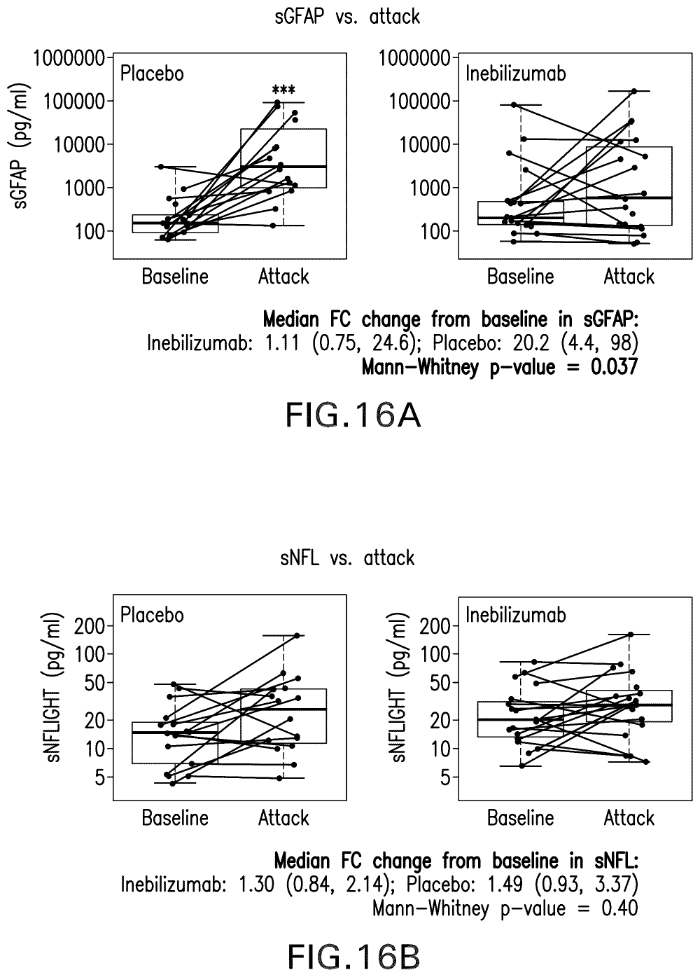

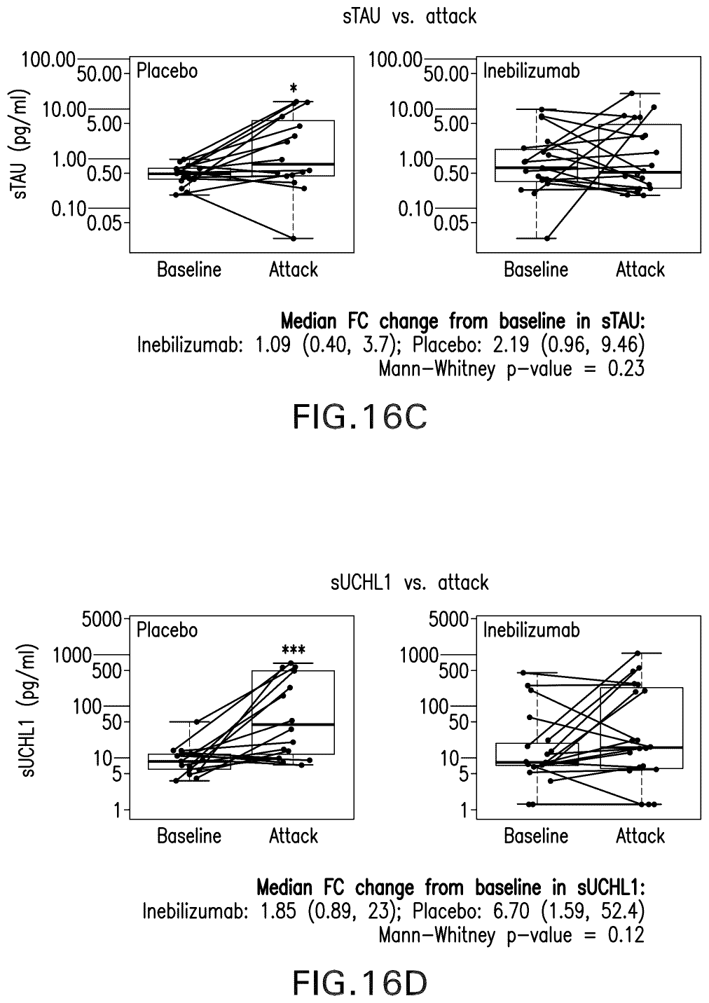

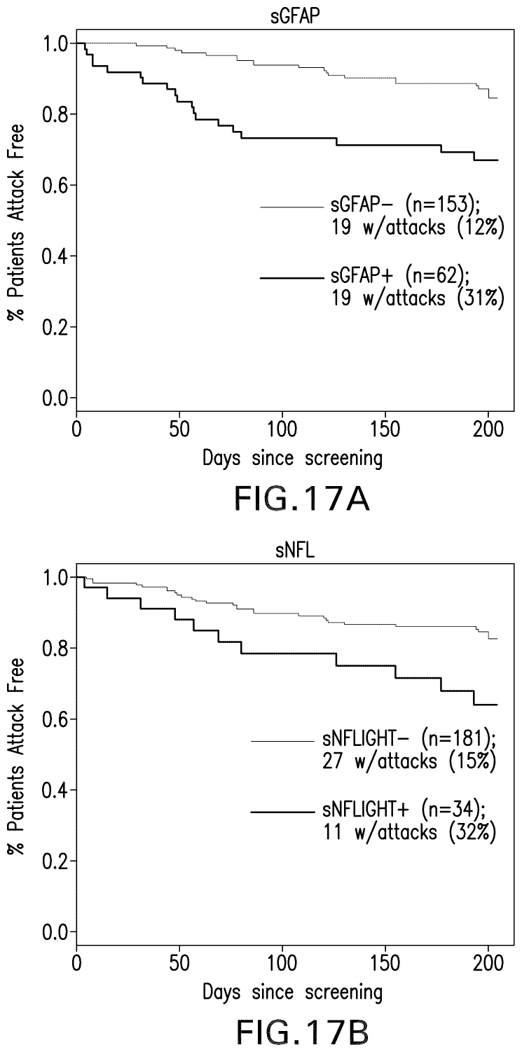

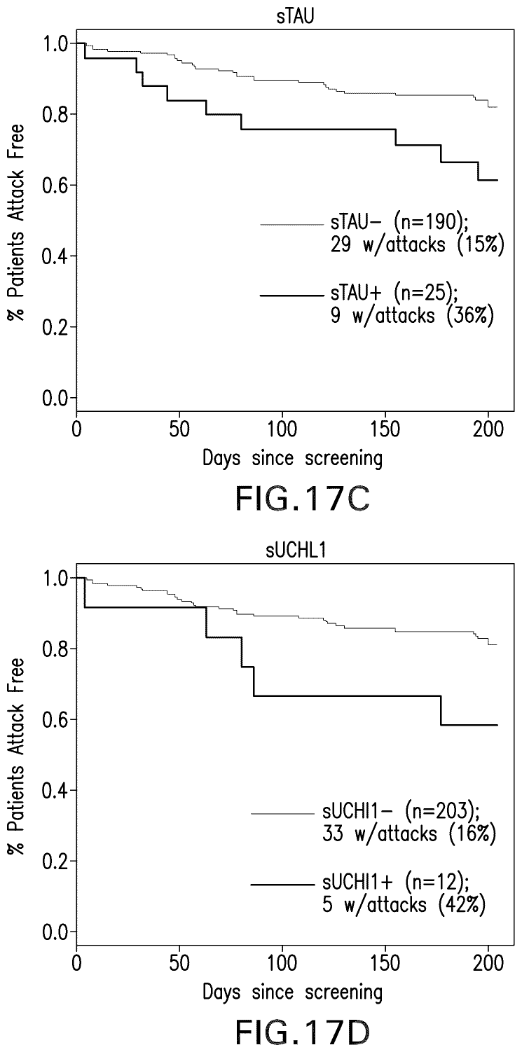

As shown in FIG. 16, all biomarker levels increased following attacks and median fold-increases from baseline (95% CI) trended higher with placebo than Inebilizumab, reaching significance with sGFAP: NfL, 1.49 (0.93-3.37) versus 1.30 (0.84-2.14), p=0.4; UCH-L1, 6.70 (1.59-52.4) versus 1.85 (0.89-23), p=0.12; Tau, 2.19 (0.96-9.46) versus 1.09 (0.40-3.7), p=0.23; sGFAP, 20.2 (4.4-98) versus 1.11 (0.75-24.6), p=0.037. As shown in FIG. 17, the baseline elevations in biomarkers were significantly correlated with increased attack risk. Baseline elevations in all biomarkers assessed cause a significant increase in the risk of an attack (sGFAP: HR, 3.03; p<0.001; sNfL: HR. 2.5; p=0.01; sTau: HR, 2.6; p=0.01; sUCHL: HR, 2.8; p=0.039).

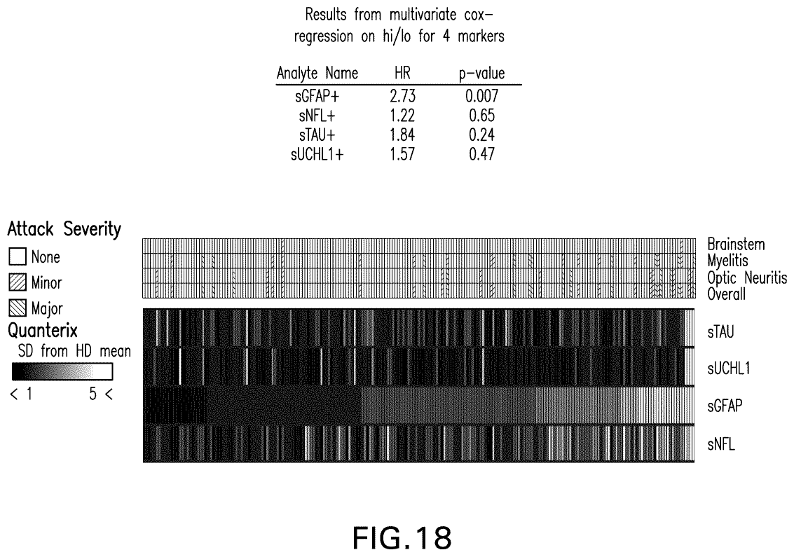

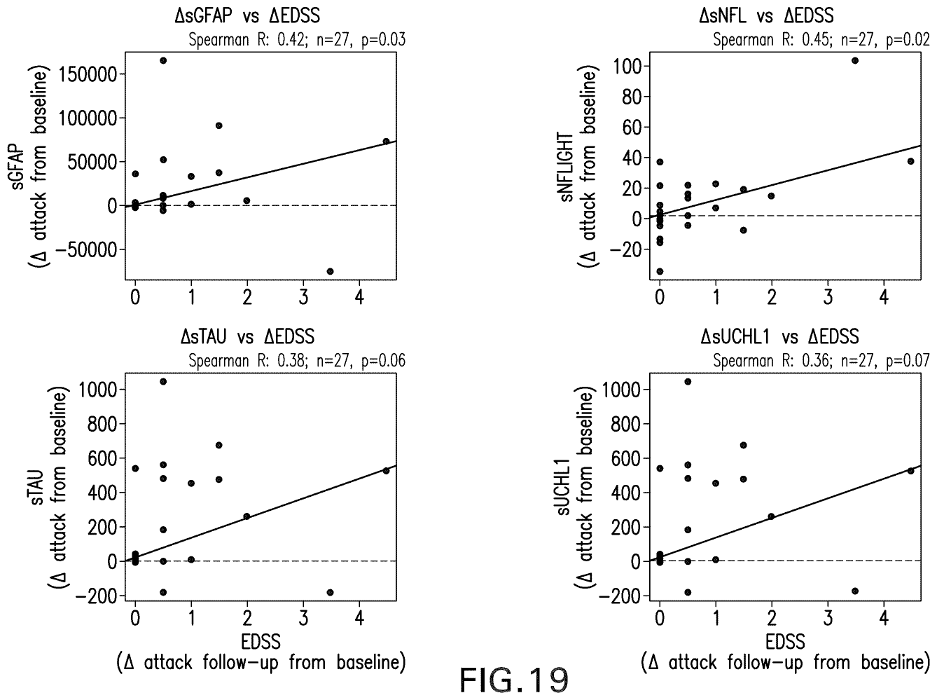

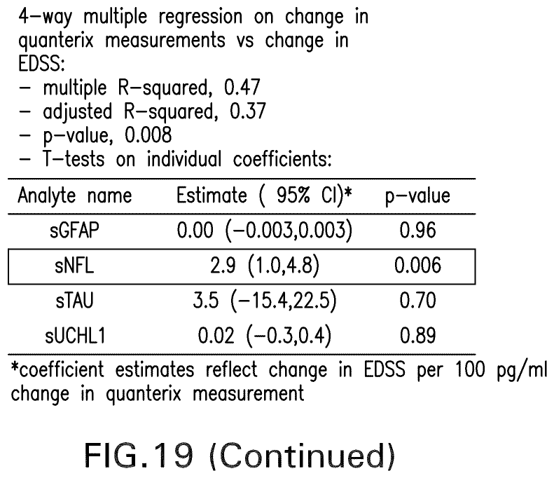

As can be seen in FIG. 18, sGFAP baseline-controlled regression analysis demonstrated that biomarkers other than sGFAP were not independently associated with attack risk. Subjects with high sTAU, sUCHL1 and sNfL tended towards highest levels of sGFAP. Cox regression analysis controlling for sGFAP concentration levels revealed that markers other than sGFAP were not independently associated with increased attack risk (hazard ratios <2, p>0.05). Following attacks, NfL correlated with EDSS score at attack assessments (R, 0.55; p<0.001); other biomarkers did not correlate with EDSS after controlling for NfL levels. As can be seen in FIG. 19, sNfL at attack is strongest correlate of EDSS change at attack follow-up (EDSS assessment and serum sample draw performed within 7 days of attack). In NMOSD, serum NfL, UCH-L1 and Tau levels were higher than in HC; increased baseline sGFAP levels were associated with a greater risk of attack. While GFAP levels showed the greatest increase following attacks, NfL correlated with attack-related disability.

1-59. (canceled)

60. A method of treating neuromyelitis optica spectrum disorder (NMOSD) in a subject in need thereof, the method comprising administering a therapeutically effective amount of a B cell depleting therapy to the subject in need thereof, wherein a sample of the subject has an elevated glial fibrillary acidic protein (GFAP) concentration.

61. The method of claim 60, wherein the elevated GFAP concentration is ≥2 standard deviations above a healthy donor mean concentration.

62. The method of claim 60, wherein the elevated GFAP concentration is at least about 170 pg/mL.

63. The method of claim 60, wherein the administration reduces: (a) a number of NMOSD-related attacks in the subject in need thereof after the administering as compared to a baseline number of NMOSD-related attacks in the subject in need thereof before the administering; or (b) a number of NMOSD-related attacks in the subject in need thereof after the administering as compared to an otherwise comparable control subject lacking the administering.

64. The method of claim 60, wherein the administration is effective at reducing: (a) a number of magnetic resonance imaging (MRI) lesions; (b) a rate of increase in new MRI lesions; or (c) both (a) and (b).

65. The method of claim 60, wherein the administration is effective at: (a) reducing a rate of worsening of expanded disability status scale (EDSS) score; or b) improving the EDSS score.

66. The method of claim 60, wherein the administration is effective at preventing NMOSD relapse in the subject as determined by: (a) a reduction in a number of MRI lesions; (b) a reduction in size of MRI lesions; or (c) both (a) and (b).

67. The method of claim 60, wherein the administration is effective at suppressing an NMOSD-related attack in the subject.

68. The method of claim 67, wherein the subject is administered a second therapeutic selected from the group consisting of: a steroid, plasmapheresis, immunoadsorption, and a complement inhibitor.

69. The method of claim 67, wherein the subject is administered a second therapeutic selected from the group consisting of: Eculizumab, Satralizumab, Ublituximab, Ravulizumab, Rituximab, Azathioprine, Mycophenolate Mofetil, a low dose corticosteroid, and any combination thereof.

70. The method of claim 60, wherein the B cell depleting therapy is selected from the group consisting of an: anti-CD19 antibody, anti-CD20 antibody, and an anti-CD22 antibody.

71. The method of claim 70, wherein the B cell depleting therapy is the anti-CD19 antibody.

72. The method of claim 71, wherein the anti-CD19 antibody comprises Inebilizumab.

73. The method of claim 72, wherein the Inebilizumab is administered at a dose of 300 mg.

74. The method of claim 60, wherein the administration is repeated every six months.

75. A method of treating neuromyelitis optica spectrum disorder (NMOSD) in a subject in need thereof, the method comprising administering a therapeutically effective amount of Inebilizumab to the subject in need thereof, wherein a serum sample of the subject has an elevated concentration of a biomarker selected from the group consisting of: glial fibrillary acidic protein (GFAP), neurofilament light chain (NfL), ubiquitin carboxyl-terminal hydrolase L1 (UCH-L1), and TAU.

76. The method of claim 75, wherein the biomarker is GFAP.

77. The method of claim 75, wherein the biomarker is TAU.

78. The method of claim 75, wherein the biomarker is UCHL1.

79. The method of claim 75, wherein the biomarker is NfL.

80. The method of claim 75, wherein the administering comprises 300 mg of Inebilizumab administered every six months.

81. The method of claim 75, wherein prior to the administration, the subject is selected as comprising the elevated concentration of the biomarker.

82. The method of claim 75, wherein the administration is effective at reducing: (a) a number of NMOSD-related attacks in the subject in need thereof after the administering as compared to a baseline number of NMOSD-related attacks in the subject in need thereof before the administering; or (b) a number of NMOSD-related attacks in the subject in need thereof after the administering as compared to an otherwise comparable control subject lacking the administering.

83. The method of claim 75, wherein the administration is effective at reducing: (a) a number of magnetic resonance imaging (MRI) lesions; (b) a rate of increase in new MRI lesions; or (c) both (a) and (b).

84. The method of claim 75, wherein the administration is effective at: (a) reducing a rate of worsening of expanded disability status scale (EDSS) score; or b) improving the EDSS score.

85. The method of claim 75, wherein the administration is effective at preventing NMOSD relapse in the subject as determined by: (a) a reduction in a number of MRI lesions; (b) a reduction in size of MRI lesions; or (c) both (a) and (b).

86. The method of claim 75, wherein the administration is effective at suppressing an NMOSD-related attack in the subject.

87. The method of claim 76, wherein the elevated GFAP concentration is ≥2 standard deviations above a healthy donor mean concentration.

88. The method of claim 75, wherein the serum sample is positive for all of GFAP, NfL, UCH-L1, and TAU.

89. A method of treating neuromyelitis optica spectrum disorder (NMOSD) in a subject in need thereof, the method comprising administering Inebilizumab to a subject in need, wherein a sample of the subject is determined to have an elevated concentration of a biomarker of neuronal injury.

90. The method of claim 89, wherein the biomarker of neuronal injury is selected from the group consisting of: glial fibrillary acidic protein (GFAP), neurofilament light chain (NfL), ubiquitin carboxyl-terminal hydrolase L1 (UCH-L1), and TAU.
